(19) United States
(12) Patent Application Publication
(10) Pub. No.: US 2023/0087578 A1
(43) Pub. Date: Mar. 23, 2023

(54) DEVICE AND METHODS FOR ENGINEERING 3D COMPLEX TISSUES

(71) Applicants: The Johns Hopkins University, Baltimore, MD (US); University of Washington, Seattle, WA (US)

(72) Inventors: Deok-Ho KIM, Baltimore, MD (US); Alex JIAO, Seattle, WA (US); Nisa P. WILLIAMS, Seattle, WA (US)

(21) Appl. No.: 17/800,494

(22) PCT Filed: Feb. 13, 2021
(86) PCT No.: PCT/US21/18067

§ 371 Date: Aug. 17, 2022

Related U.S. Application Data
(60) Provisional application No. 62/978,235, filed on Feb. 18, 2020.

(51) Int. Cl. A61L 27/38; A61K 35/12; A61K 35/34; A61L 27/52; C12N 5/077; C12N 5/071

(52) U.S. Cl. CPC A61L 27/38; A61K 35/12; A61K 35/34; A61L 27/52; C12N 5/0657; C12N 5/0658; C12N 5/0691; C12N 5/0697; C12N 2506/45; C12N 2539/10

(57) ABSTRACT

Provided herein is a method for making a tissue engineering scaffold. The method includes layering at least one sheet of cells onto a flexible scaffold, casting the sheets into geometries, and thereby creating the tissue engineering scaffold. Preferred geometry are non-linear (i.e. not a substantially flat surface such as may be provided by a flat glass substrate). The flexible scaffold is characterized by tensile strength, viscosity, stress, strain, modulus of polymers, or any combination thereof.

### RELATED APPLICATIONS

This application claims the benefit of priority under 35 U.S.C. § 119(e) to U.S. Provisional Application No. 62/978,235, filed Feb. 18, 2020, the entire contents of which is incorporated herein by reference in its entirety.

### STATEMENT AS TO FEDERALLY SPONSORED RESEARCH

This invention was made with government support under Grant Nos. HL126445, HL145809, HL146436, NS094388, and AR064395 awarded by the National Institutes of Health (NIH). The government has certain rights in the invention.

### BACKGROUND

New devices and methods for engineering 3D complex tissues is needed.

### BRIEF SUMMARY

In aspects, provided herein is a method for making a tissue engineering scaffold. The method includes layering at least one sheet of cells onto a flexible scaffold, casting the sheets into geometries, and thereby creating the tissue engineering scaffold. Preferred geomstry are non-linear (i.e. not a substantially flat surface such as may be provided by a flat glass substate.)

In embodiments, the flexible scaffold is characterized by tensile strength, viscosity, stress, strain, modulus of polymers, or any combination thereof. For example, the flexible scaffold of the present disclosure has a tensile strength can be measured using tensile stiffness (e.g., in a range from about 20-40 kpis (kg/mm²). Flexibility can further be measured by the tensile modulus of elasticity or a measurement of elongation at break range. The tensile modulus of elasticity for PET (polyester) films can range from about 300,000 psi to about 600,000, specifically from about 400,000 psi to about 500 psi, or about 480,000 psi. In embodiments, the elongation at break range (e.g., a fracture break) is the ratio between changed length and initial length after breakage of the test specimen. It expresses the capability of natural plant fiber to resist changes of shape without crack formation. A percentage of the original length is used to express the elongation at break. It is typically in the order of 100-600%, and some examples even go up to 1000%. As used herein, the elongation at break is from about 100% to about 250%, or from about 110% to about 210%.

In embodiments, the methods provide that the at least one sheet of cells has a continuous sheet of cells, e.g., a single continuous sheet of cells. In other embodiments, the flexible scaffold is thermoresponsive. For example, in one aspect, the temperature range is such that at about 30° C. or less, the flexible scaffold detaches; and at about greater than 30° C. (such as about 30.5° C. or 31° C. or greater), the flexible scaffold attaches. In another aspect, the temperature range is such that at about 31° C. or less, the flexible scaffold detaches; and at about greater than 31° C. (such as about 31.5° C. or 32° C. or greater), the flexible scaffold attaches. In another aspect, the temperature range is such that at about 32° C. or less, the flexible scaffold detaches; and at about greater than 32° C. (such as about 32.5° C. or 33° C. or greater), the flexible scaffold attaches. In another aspect, the temperature range is such that at about 33° C. or less, the flexible scaffold detaches; and at about greater than 33° C. (such as about 33.5° C. or 34° C. or greater), the flexible scaffold attaches. In another aspect, the temperature range is such that at about 34° C. or less, the flexible scaffold detaches; and at about greater than 34° C. (such as about 34.5° C. or 35° C. or greater), the flexible scaffold attaches. In another aspect, the temperature range is such that at about 35° C. or less, the flexible scaffold detaches; and at about greater than 35° C. (such as about 35.5° C. or 36° C. or greater), the flexible scaffold attaches. In another aspect, the temperature range is such that at about 36° C. or less, the flexible scaffold detaches; and at about greater than 36° C. (such as about 36.5° C. or 37° C. or greater), the flexible scaffold attaches. In another aspect, the temperature range is such that at about 37° C. or less, the flexible scaffold detaches; and at about greater than 37° C. (such as about 37.5° C. or 38° C. or greater), the flexible scaffold attaches. Specifically, for poly (N-isopropylacrylamide) (pNIPAM), the temperature range may be about 32° C. where the flexible scaffold detaches, and greater than 32° C. (such as 32.5° C. or 33° C.), the flexible scaffold attaches.

In other embodiments, the geometries of the tissue engineered scaffold includes tubes, cones, heart ventricular shapes cylinders, arcs, curves, hollow shapes, spheres, and the like.

In other embodiments, the sheet of cells has a monolayer of the cells. For example, the cells may be substantially confluent (e.g., greater than 75% confluent, greater than 80% confluent, or greater than 90% confluent).

In some examples, the sheet of cells are aligned in a uniform direction, e.g., in a singly uniform direction.

In other examples, the layering of the methods herein includes cell sheets from about 1 to about 10 sheets of aligned cells, or about 1-5 sheets of aligned cells, or about 2-3 sheets of aligned cells. In particular examples, as the number of sheets increases, vascularization may also be required. Vascularization, for example, may take about 1 day to about 10 days. In specific examples, the vascularization may take about 0.25, 0.5, 0.75, 1, 1.5, 2, 2.5 or 3 days, more typically 0.5, 1 or 2 days. Vascularization is characterized by the sprouting of new vessels (and the ability to form lumens). Moreover, markers such as CD31 can be used to measure vascularization

In other examples, the tissue engineering scaffold has a thickness from about 100 μm to about 400 μm. In other examples, the thickness may range from 20 μm to 100 μm, or from 100 μm to 500 μm, or from 100 μm to 400 μm, or from 100 μm to 300 μm, or from 100 μm to 200 μm. As described herein, the thickness (e.g., of about 4 to 5 cell layers may be about 300 μm to about 400 μm thick).

In other examples, the cells of the current invention include a muscle cell. Alternatively, the cells include smooth muscle cells, cardiac cells, skeletal cells, neuronal cells, cancer cells, endothelial cells, fibroblasts, chondrocytes, and combinations thereof.

In embodiments, the flexible scaffold of the current invention is capable of being twisted, folded, stacked, rolled, or wrapped.

In further embodiments, the methods described herein do not utilize elctrospinning.

In aspects, provided herein, is a tissue engineering scaffold capable of molding into a desired geometry, wherein the tissue engineering scaffold includes a flexible scaffold, a functional layer, where the functional layer comprises poly (N-isopropylacrylamide) (pNIPAM), or a derivative thereof, and a polymer.

For example, the polymer of the tissue engineering scaffold has a molecular weight of between about 200 and about 10,000 Da.

In further embodiments, the polymer is an ultraviolet-curable polymer.

In embodiments, the scaffold is a hydrogel.

In embodiments, the cells of the tissue engineering scaffold include muscle cells. In examples, the cells include smooth muscle cells, cardiac cells, skeletal cells, neuronal cells, cancer cells, endothelial cells, fibroblasts, chondrocytes, and combinations thereof.

In embodiments, the tissue engineering scaffold further includes a drug molecule, an adhesion molecule, a signaling molecule, an imaging agent For example, angiogenic and myogenic factors may be conjugated (incorporated) into the tissue engineering scaffold described herein. In specific examples, the angiogenic factor includes sphingosine-1-phosphate (S1P). S1P is a potent angiogenic and myogenic factor used to enhance myoblast and endothelial maturation. In other examples, growth factors, such as vascular endothelial growth factor (VEGF), basic fibroblast growth factor (bFGF), and platelet derived growth factor (PDGF) can be conjugated tot eh tissue engineering scaffold described herein. Additional factors include bone morphogenetic proteins (BMPs), colony stimulating factors (CSF), epidermal growth factor (EGF), insulin growth like factor (IGF), interleukins (e.g., IL1, IL2, IL3, IL4, IL5, IL6, 117, IL8, IL9, IL10, IL11, IL12, or IL13), transforming growth factor α (TGFα), transforming growth factor β (TGFβ), or nerve growth factor (NGF).

In embodiments, the functional layer of the tissue engineering scaffold includes poly (N-isopropylacrylamide) (pNIPAM).

In aspects, provided herein is a method for repair or replacement of a tissue including applying a tissue engineering scaffold, wherein the tissue engineering scaffold includes flexible scaffold, and a polymer the current invention.

In aspects, provided herein is a method for in vitro disease modeling, comprising making a tissue engineering scaffold by layering at least one sheet of aligned cells onto a flexible scaffold, casting the sheets into geometries, and thereby creating the tissue engineering scaffold, and thereby modeling the disease of interest. For example, the disease includes. cardiovascular disease, myopathy, vascular disease, or endothelial barrier disease. In other examples, the disease may include neuromuscular disease and neuropathies. Moreover, the diseases may include Parkinson's Disease, Alzheimer's Disease, dementia. Furthermore, the tissue engineering scaffold of the method herein can be used following a tumor resection surgery of a patient.

Other aspects of the invention are disclosed infra.

### DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show fabrication of anisotropic multilayered tissues using flexible thermoresponsive nanofabricated substrates (fTNFS) and nanopatterned cell sheet engineering. FIG. 1A are images showing fabrication of flexible nanopatterned substrates (fTNFS) using capillary force lithography and subsequent thermoresponsive functionalization with amineterminated pNIPAM. FIG. 1B is an image of flexible-TNFS after curing and a-PNIPAM functionalization. Rainbow coloring is caused by the nanotopography diffracting light. (Inset) Scanning electron micrograph of fTNFS surface demonstrating high fidelity fabrication of the ridge-groove nanotopography. FIG. 1C is a schematic of gel casting and stacking of organized cell monolayers from flexible TNFS. FIG. 1D is a Z-stack cross-sectional image of smooth muscle cell tri-layer tissue stack 24 h after stacking. Top and bottom sheets were membrane-dyed red (Cell Tracker Red) and middle sheet was membrane-dyed green (Cell Tracker Green) before stacking.

FIG. 2 is a schematic of tubular tissue casting process using multilayered cell-sheet stacks with fTNFS and cylindrical molds.

FIGS. 3A-3F are images showing fabrication of 3D tubular tissues with circumferential cellular alignment. FIG. 3A is an image of tissue casting implements. (i) Mandrel that is inserted through the 3D printed end cap (iii) into the cylindrical mold (ii) to create a hollow lumen through the center of the resulting tubular tissue. FIG. 1B is an image of a casting mold pieces in B assembled as during tissue casting. The fTNFS and cell layers are manipulated into the cylindrical mold (ii), the cap end (iii) is fastened over one end of the cylindrical mold (ii), and the mandrel (i) is inserted through the bottom of the end cap (iii), and the hydrogel is pipetted into the open end of the mold (ii) to cast the cell sheets around a hydrogel tube. FIG. 1C is an image of resulting tubular tissue attached to a custom 3D-printed housing in a culture well after removal from the casting mold. FIG. 1D is an image of a cross-sectional schematic of expected tissue dimensions and structure. Thickness of cell layers is dependent on cell type and number of layers. FIG. 1E is a histological cross-section of SMC tube stained with hematoxylin and eosin showing a hollow central lumen encircled by a gelatin hydrogel layer and cell layers. FIG. 1F is a boxed inset of (FIG. 1E) demonstrating three layers of SMCs with elongated nuclei along the curvature of the tube's outer edge.

FIGS. 4A-4D shows fabrication of patterned 3D tubular tissues with three muscle cell types. FIG. 4A are images of a 3D rendered image of a confocal z-stack of a smooth muscle cell tube. Image was rotated to show the curvature of the tubular tissue's outer surface. FIG. 4B is a boxed inset of (FIG. 4A) showing a cross-sectional view of confocal z-stack demonstrates cell layers are wrapped around the tube's outer edge of the hydrogel. FIG. 4C is an image showing the maximum intensity projection of a confocal z-stack taken of a tubular tissue circumferentially patterned with mouse muscle myoblasts (C2C12s) and cultured in differentiation medium to promote fusion of myoblasts into elongated myotubes (MYH, all isoforms). FIG. 4D is a maximum intensity projection of a confocal z-stack taken of a tubular tissue circumferentially patterned with iPSC-derived cardiomyocytes. A further image showed global circumferential cellular alignment perpendicular to the tube's long axis. Dashed white lines outline the edges of the tubular constructs in (FIG. 4C) and (FIG. 4D).

FIGS. 5A-5I show patterned cellular orientation in tubular tissues is maintained after 7 days in culture. (FIGS. 5A-5C) Brightfield images of smooth (FIG. 5A), skeletal (FIG. 5B), and cardiac (FIG. 5C) muscle tubes after 7, 14, or 7 days in culture, respectively. (FIGS. 5D-5F) Confocal images of each tubular tissues imaged in FIGS. 5A-5C, respectively. Each tissue was immunostained for cytoskeletal and or contractile proteins as listed in the upper right corner of each panel. (FIGS. 5G-5I) Quantitative analysis of filamentous-actin (F-actin) cytoskeletal alignment of cells in each tissue type.

FIGS. 6A-6B are images showing representative brightfield microscope images of detaching nanopatterned cardiac sheets without incorporation of stromal cells (FIG. 6A) and with incorporation of endothelial cells (FIG. 6B) demonstrating intact, spontaneous cardiac sheet detachment only in the coculture condition. Direction of cell sheet detachment is labeled by the black arrow. Double headed yellow arrows in (FIG. 6A) and (FIG. 6B) denote the orientation of the nanotopography on the scaffold below. Scale bars, 100 μm.

FIG. 7 is a graph showing flow cytometry of purified iPSC-derived cardiomyocytes stained or cardiac troponin T (cTnT). Cells were subjected to lactate selection medium for 3 days and then harvested on day 17 for flow cytometry. 99.2% of cells were identified as positive for cTnT.

FIG. 8 is a graph showing flow cytometry of iPSC-derived endocardial-like endothelial cells livestained for CD31 surface markers on day 12. 91.0% of cells were identified as positive for CD31 surface markers.

FIGS. 9A-9C are images showing a bioinspired design and implementation of a flexible thermoresponsive, nanostructured substrate to engineer organized cardiac tissues and organoids. FIG. 9A is a schematic illustration of helically structured myocardial sheets in the heart, which are organized in anisotropic cardiac layers comprised of aligned cardiomyocytes and extracellular matrix fibers 4-6 myocytes thick. FIG. 9B is a schematic illustration of cardiac cell sheet engineering using the thermoresponsive nanostructured substrate (TNFS) to engineer aligned cardiac cell sheets, detach cardiac cell sheets into 4-layered modular laminae, which can then be further stacked to engineer thick, structured cardiac tissues. FIG. 9C is a schematic illustration of stacked modular laminae to engineer thick, helical cardiac tissues.

FIGS. 10A-10D are data showing tissue and substrate parameters to engineer anisotropic cardiac sheets. FIG. 10A are representative brightfield microscope images of TNFS with varying GMA percentages, affecting PNIPAM grafting density and monolayer formation. Scale bars, 100 μm. FIG. 10B is a graph of flow cytometry analysis of cTnT+ cells before and after metabolic purification demonstrating a 3-fold increase in cTnT+ cells after selection. FIG. 10C are representative brightfield microscope images of cardiac sheets with unpurified, hiPS-CMs (top) and metabolically purified hiPS-CMs (bot) on 1% GMA TNFS, demonstrating increased alignment and syncytial monolayer formation with incorporation of purified cardiomyocytes. Scale bars, 100 μm. FIG. 10D are representative brightfield microscope images of detaching nanopatterned (NP) cardiac sheets without incorporation of stromal cells (top) and with incorporation of 20% endothelial cells (bot) demonstrating intact, spontaneous cardiac sheet detachment only in the presence of an ECM producing stromal cell coculture. Scale bars, 100 μm.

FIGS. 11A-11C are data showing nanopatterned endocardial-cardiomyocyte (cardiac) cocultured sheets can be transferred to other surfaces while maintaining alignment and deposited extracellular matrix proteins. FIG. 11A is a brightfield microscope image showing the spontaneously detachment of anisotropic cardiac sheet at 22° C. on a TNFS. Scale bar, 50 μm. FIG. 11B is a confocal microscope image of an immunofluorescently labeled cardiac cell sheet 7 days after transfer to a glass coverslip demonstrating maintained cytoskeletal alignment long-term, well-ordered sarcomeric arrays, and organized cell-deposited extracellular matrix proteins. Scale bar, 100 μm, inset, 10 μm. FIG. 11C is a quantitative assessment of cytoskeletal alignment of both nanopatterned (NP) cardiac sheets and unpatterned (UP) controls illustrating the degree of alignment in NP cardiac sheets 7 days post-transfer to a glass coverslip.

FIGS. 12A-12F are data showing aligned, 4-layer cardiac tissues maintain discrete layers and overall tissue alignment. FIG. 12A is a schematic representation of the fluorescently labeled cell sheets stacked in a RGRG configuration with uniaxial alignment. FIG. 12B is a confocal microscope image of CellTracker labeled cardiac cell sheet 1 day after stacking and transfer to a glass coverslip demonstrating maintained tissue alignment (top) while also maintaining discrete, individual layers (bottom). Scale bar top, 100 μm, bottom, 40 μm. FIG. 12C is a confocal microscope z-stack 3D-rendering of the immunofluorescently stained cardiac sheet demonstrating cytoskeletal alignment (top) and overall cell-dense, 3D tissue thickness (bottom). Scale bar, 40 μm. FIG. 12D is a graph of a quantitative assessment of cytoskeletal alignment throughout the 4-layer cardiac tissue, demonstrating maintained uniaxial alignment throughout the tissue and individual layers. FIG. 12E is a representative image of an immunofluorscently stained individual cardiac sheet demonstrating cytoskeletal alignment, well-ordered sarcomere structures, and presence of cell-deposited extracellular matrix. FIG. 12F are representative frames during CCQ analysis of cardiac tissue contractions with motion vectors overlayed, demonstrating unidirectional motion of aligned 4-layer cardiac tissues during contraction.

FIGS. 13A-13E are data showing helical, 4-layer cardiac tissues maintain discrete layers and overall tissue alignment. FIG. 13A is a schematic representation of the helically-stacked 4-layer cardiac tissue. FIG. 13B is a confocal microscope z-stack 3D-rendering of the immunofluorescently stained helical cardiac sheet. Scale bar, 40 μm. FIG. 13C is a quantitative assessment of cytoskeletal alignment throughout the 4-layer cardiac tissue, demonstrating discrete layer alignments. FIG. 13D are representative images of immunofluorscently stained individual cardiac sheet demonstrating layer-dependent, cytoskeletal alignment. FIG. 13E are representative frames during CCQ analysis of cardiac tissue contractions with motion vectors overlayed, demonstrating swirling motion of helical 4-layer cardiac tissues during contraction.

FIGS. 14A-14D are data showing the structural organization of 3D cardiac tissue improves contractile function. CCQ-based quantification of contraction videos of nanopatterned (NP) cardiac sheets and unpatterned (UP) controls demonstrating increased contraction magnitude (FIG. 14A), velocity (FIG. 14B), and relaxation velocity (FIG. 14C). FIG. 14D shows the representative average contraction angle histograms of NP cardiac sheets and unpatterned control.

FIGS. 15A-15C are data showing the metabolic purification of hiPS-derived cardiomyocytes yields high purity cell cultures. FIG. 15A are microscope images showing the loss of non-cardiomyocyte cells due to metabolic purification in during cell culture over the course of 7 days. Scale bars, 100 μm. FIG. 15B shows flow cytometry analysis of cTnT+ cells before and after metabolic purification demonstrating a 3-fold increase in cTnT+ cells after selection. FIG. 15C is an image showing immunofluorescent confirmation of metabolically purified cardiomyocytes. α-sarcomeric actin in red, phalloidin in green, Hoechst in blue. Scale bar, 200 μm.

FIG. 16 are images showing that PNIPAM grafting density affects formation of anisotropic cardiac monolayers. Brightfield microscope image demonstrating varying degrees of cardiac monolayer and subsequent sheet formation dependent on the concentration of GMA copolymer used in the TNFS. Scale bars, 200 μ.

FIGS. 17A-17D are images showing that pure cardiac cell sheets do not detach as intact, anisotropic cell sheets. FIG. 17A is an image showing that 99% cTnT+ (purified) cardiomyocytes seeded on a 1% GMA TNFS show formation of elongated, aligned cardiomyocytes 24 hours after seeding. FIG. 17B is an image showing that at D7 (7 days post seeding) cardiomyocytes form a syncytial monolayer with maintained alignment. FIG. 17C is an image showing the reduction of culture temperature from 37 C to 22 C causes cells to detach from the surface but not as an intact sheet. FIG. 17D is an image that transferred pure cardiac cell sheets lose initial alignment and are only partially transferred.

FIG. 18 is a confocal microscope image demonstrating alignment of structural proteins and contractile apparatus in pure cardiac cell sheets seeded onto a TNFS. Confocal microscope image of immunofluorescently labeled pure cardiac cell sheets. α-sarcomeric actinin in red, phalloidin in green, Hoechst in blue. Scale bar, 50 μm.

FIGS. 19A-19D are images showing that stromal cell coculture with cardiomyocytes to engineer detachable, anisotropic cardiac cell sheets. FIG. 19A is a brightfield microscope image showing incomplete cardiac cell sheet formation during coculture of the hs5 stromal cell line and cardiomyocytes. FIGS. 19B-19D are brightfield microscope image showing formation of aligned cardiac cell sheets during coculture of the hs27a stromal cell line (FIG. 19B), primary human dermal fibroblasts (FIG. 19C), and hiPS-derived hemogenic anterior endocardial-like endothelial cells (FIG. 19D, ECs). All stromal cells mixed in a 1:5 ratio with cardiomyocytes. Scale bars, 100 μm.

FIGS. 20A-20C are images showing that endocardial cells (ECs) demonstrate best formation and transfer of aligned cardiac cell sheets. FIG. 20A is a confocal microscope image of immunofluorescently stained, transferred hs27a cocultured cardiac cell sheets, demonstrating lost alignment after transfer to a glass surface. FIG. 20B is a confocal microscope image of immunofluorescently stained, transferred hDF cocultured cardiac cell sheets, demonstrating but asyncytial beating with uneven distribution of cardiomyocytes. FIG. 20C is a confocal microscope image of immunofluorescently stained, transferred EC cocultured cardiac cell sheets, demonstrating but well-aligned, syncytial monolayers. α-sarcomeric actinin in red, phalloidin in green, Hoechst in blue. Scale bar, 200 μm.

FIGS. 21A-21C are images showing that engineered, anisotropic cardiac sheets can undergo sheet mixing and reorganization while maintaining alignment. FIG. 21A is a confocal microscope z-stack image of red and green membrane-labeled, 4-layer aligned cardiac sheets, demonstrating layer mixing between sheets from bottom (upper left) to top (bottom right). Scale bar, 100 μm. FIG. 21B is a confocal microscope z-stack 3D-rendering of the membrane-labeled cardiac sheet demonstrating intermixed red and green cardiac cells. FIG. 21C is a high resolution image demonstrating mixed red and green cells within a single layer. Scale bar, 100 μm.

FIGS. 22A-22J are data showing design and fabrication of cardiac ventricular models. FIG. 22A is an image inspired by the layered organization of the myocardium, modeling three main cellular organizations in this study: longitudinal (90°), angled (45°), and circumferential) (0°). FIG. 22B (left) is an illustration of flexible thermoresponsive nanofabricated substrates (TNFS) with direction of nanogrooves denoted (θ°). FIG. 22B (right) Experimental timeline of serial cell seeding onto flexible TNFS for thick organized cell sheets. FIG. 22C is a schematic of 3D ventricle model fabrication from organized cardiac sheets on fTNFS using custom molds and fibrin hydrogel (FIG. 26). FIG. 22D is a representative image of engineered ventricular model attached to a tissue mount in culture with tissue edge outlined in green dashed line. Tissues exhibited spontaneous contractions within one hour after removal from the molds. FIG. 22E is a 3D confocal z-stack projection of a circumferentially patterned ventricular model immediately after fabrication (day 0). The tissue was stained for sarcomeric protein titin (magenta) and filamentous actin fibers (F-actin, green). FIG. 22F is a schematic of ventricular model cross-section highlighting inner fibrin wall covered by outer cell layers. FIG. 22F (right) Overview of confocal imaging scheme to image through tissue wall from outer to inner cell layers. Each layer was analyzed separately for cellular alignment angle. FIG. 22G-J are 3D confocal z-stacks of cardiac ventricular models fabricated with (FIG. 22G) isotropic (random), (FIG. 22H) circumferential (0°), (FIG. 22I) angled (45°), and (FIG. 22J) longitudinal (90°) cellular patterning. Cellular alignment is demonstrated by f-actin (green) organization in each model.

FIGS. 23A-23F show the quantification of cellular alignment in 3D cardiac ventricular models over time. 3D confocal z-stack projections of ventricular models patterned with (FIG. 23A) isotropic, (FIG. 23B) circumferential (0°), (FIG. 23C) angled (45°), and (FIG. 23D) longitudinal (90°) cellular organization as demonstrated by the filamentous actin organization (F-actin, grey). Representative images are from different tissues fixed and imaged on day 0 (immediately after fabrication) and day 4 of culture. All scale bars are 50 μm. (Right of each image) Polar histograms representing the distribution of cellular alignment of the inner (pink) and outer (blue) cell layers of each tissue organization (FIGS. 23A-D), respectively. The area of each bar is the number of observations for that orientation angle relative to the total number of observations per image. Red lines on each histogram represent the mean orientation angle for the specific image on its left but does not represent the mean of the group. (FIG. 23E) Average cellular orientation angles for outer (blue) and inner (pink) layers of each tissue group on day 0 and 4, respectively. (FIG. 23F) Average mean resultant vector length (RVL) for outer (blue) and inner (pink) layers of each tissue group on day 0 and 4, respectively. A RVL value closer to 1 can be interpreted as greater cellular organization towards the mean orientation angle for that tissue. Each data point in (FIGS. 23E and 23F) represents the mean orientation angle of one tissue. (FIGS. 23E and 23F) Error bars represent the standard error from the mean. *p<0.01, **p<0.001, ***p<0.0001.

FIGS. 24A-24E show 3D Finite element (FE) model of transmural shear stress and strain. FIG. 24A shows 3D rendering of FE model of a longitudinally (cellular orientation=90°) patterned tissue. The color map projected onto the model represents the range of tissue deformation or movement at peak systole. FIG. 24B is a schematic of how transmural shear stress and strain were measured in (C-E) where r=radial distance from the fibrin wall at the lumen space. Inner and outer cell layers are depicted in pink or blue, respectively. (FIG. 24C) Longitudinal shear stress, (FIG. 24D) longitudinal strain, and (FIG. 24E) circumferential strain measured across a tissue's wall thickness as illustrated in (FIG. 24B) for all organizational groups. Yellow, pink and blue panels highlight the thickness and position of the fibrin wall, and the inner and outer cell layers, respectively.

FIGS. 25A-25F are data showing a functional assessment of ventricular models thorough isovolumic pressure production. FIG. 25A is an image of ventricular model under catheterization during live pressure recordings. Tissues are positioned upright on a 3D printed stage during recordings. Culture medium was removed for a clearer view. FIG. 25B is a representative pressure trace from a tissue under 1 Hz electrical field stimulation with 3 volt, 10 millisecond pulses. FIG. 25C is a bar graph showing average contractile pressure amplitude from each tissue organization. Each data point represents one tissue within a group. FIG. 25D is a bar graph showing average contraction (top) and relaxation (bottom) velocities for each tissue group as measured by the change in pressure over the change in time (dP/dt). FIG. 25E is a bar graph showing the average spontaneous (without electrical stimulation) beat frequencies recorded from several tissues within each group. FIG. 25F is a bar graph showing the average maximum pacing frequencies or capture rates for each group. (FIG. 25C-25F) All measurements were taken on day 4 of tissue culture. Error bars represent the standard error from the mean. *p<0.05; **p<0.01; ***p<0.0001.

FIG. 26 is a schematic of the assembly of custom conical molds for 3D ventricular tissue fabrication. 3D rendered models of tissue casting mold pieces and their assembly order. (i) The bottom mold piece is used as a platform to assemble the two base pieces (ii). The base pieces join to create a conical lumen into which the fTNFS and cell sheets are folded and inserted into. A mid piece or tissue mount (iii) is inserted over top of the conical hole to prevent the fTNFS and cell sheets inside from springing out. The fibrin hydrogel is pipetted into the opening at this step. Finally, the top piece (iv) acts as a positive mold to create a hollow lumen in the final tissue.

FIG. 27 is an image of the long-axis cross-sectional view of an engineered cardiac ventricular model using brightness mode echocardiography.

FIG. 28 is data showing the analysis of engineered cardiac ventricular model function using brightness-mode echocardiography. The left-ventricular trace function is used to track the motion of the inner walls of the ventricular model. Changes in chamber geometry each contraction and relaxation (e.g. area, volume) are used to calculate cardiac function (e.g. beats per minute, ejection fraction, fractional shortening, and cardiac output (mL fluid/min)). Electrical field stimulation pulses used to stimulate the cardiac ventricular model are detectable with the echocardiography system and can be timed with the corresponding contractions of the ventricular model.

FIG. 29 is a schematic of a method of cell sheet stacking using cells sheets grown on flexible TNFS.

### DETAILED DESCRIPTION

Provided herein are, inter alia, are methods and compositions for making a tissue engineering scaffold. The method includes layering at least one sheet of cells onto a flexible scaffold, casting the sheets into geometries, and thereby creating the tissue engineering scaffold.

Tissue engineering aims to capture the structural and functional aspects of diverse tissue types in vitro. However, most approaches are limited in their ability to produce complex 3D geometries that are essential for tissue function. Tissues, such as the vasculature or chambers of the heart, often possess curved surfaces and hollow lumens that are difficult to recapitulate given their anisotropic architecture. Cell-sheet engineering techniques using thermoresponsive substrates provide a means to stack individual layers of cells with spatial control to create dense, scaffold-free tissues.

Provided herein is a method to fabricate complex 3D structures by layering multiple sheets of aligned cells onto flexible scaffolds and casting them into hollow tubular geometries using custom molds and gelatin hydrogels. To enable the fabrication of 3D tissues, a thermoresponsive nanopatterned cell-sheet technology was used by applying it to flexible substrates that could be folded as a form of tissue origami. The versatile nature of this platform was demonstrated by casting aligned sheets of smooth and cardiac muscle cells circumferentially around the surfaces of gelatin hydrogel tubes with hollow lumens. Additionally, skeletal muscle was patterned in the same fashion to recapitulate the 3D curvature that is observed in the muscles of the trunk. The circumferential cell patterning in each case was maintained after one week in culture and even encouraged organized skeletal myotube formation. Additionally, with the application of electrical field stimulation, skeletal myotubes began to assemble functional sarcomeres that could contract. Cardiac tubes could spontaneously contract and be paced for up to one month. The flexible cell-sheet engineering approach provides an adaptable method to recapitulate more complex 3D geometries with tissue specific customization through the addition of different cell types, mold shapes, and hydrogels. By enabling the fabrication of scaled biomimetic models of human tissues, this approach could be used to investigate tissue structure-function relationships, development, and maturation in the dish.

### Tissue Engineering

Tissues throughout the body possess complex three-dimensional (3D) structures with many degrees of organization and function. For example, the vasculature, like many other tissues, is organized by stratification of several layers of different cell types that perform complementary functions to modulate blood pressure and tissue perfusion^{[1,2]}. The endothelial cells in the lining of the blood vessel's lumen are oriented parallel to the direction of blood flow, whereas the surrounding smooth muscle cells that encircle the endothelium are aligned perpendicularly. Similar patterns of differential organization are observed in the helical fiber organization of the myocardium in the heart and in the radial fan patterns seen in the trapezius and pectoral muscles of the trunk. The function of each of these tissues is highly dependent upon their structure and 3D geometry, and when their organization is compromised by disease it can be detrimental or potentially fatal^{[3-5]}.

To study tissue function and their associated diseases, advancements have been made in tissue engineering to recapitulate tissue micro- and macroenvironments in vitro. For example, cell-dense cardiac tissue patches made from induced pluripotent stem cell-derived cardiomyocytes (iPSC-CMs) can mimic action potential conduction velocities close to those of adult cardiac tissues^{[6-8]}; vascular grafts have been made from cell-deposited matrix and then decellularized before implantation^{[9-11]}; and bioprinting with cellularized-inks (or bioinks) has enabled fabrication of intricate 3D tissue-specific structures with corresponding function^{[12-15]}. A challenge facing each of these approaches is that tissues often have complex 3D geometries, including curved surfaces and hollow lumens. Such structures have been difficult to recreate in vitro due to limitations of available fabrication techniques. Specifically, there are few fabrication approaches that allow for production of curved 3D geometries while also having control over spatial organization at the cell-layer level. The ability to recapitulate these structures would impart function that better mimics native tissues and organs.

To address this need, a nanofabrication technique was established to pattern sheets of organized cells and stack them to create multi-layered tissue patches using a novel gel-casting technique in conjunction with thermoresponsive substrates^{[16,17]}. This technology introduces flexible substrates and custom molds to enable the fabrication of organized 3D tissue structures. Multiple cell types could be patterned to form an intact monolayer with a uniform orientation in the direction of the nanotopography. Each monolayer was lifted from the surface through temperature-mediated release provided by the thermoresponsive poly (N-isopropylacrylamide) (pNIPAM) functional layer. Multiple organized monolayers were stacked onto a single flexible film and were folded into a cylindrical shape, as a form of tissue origami, where the organized cell layers could be casted into a free-standing 3D tubular tissue. The diverse application of this technology by fabricating tubular tissues with curved surfaces from three muscle cell types: smooth, skeletal, and cardiac was demonstrated. This approach enabled patterning of all three cell types in 3D multilayered tissues with circumferential alignment that was maintained for two weeks in culture. Additionally, with application of electrical field stimulation, skeletal myotubes assembled functional sarcomeres that could contract, and cardiac tubes could be paced for over one month. This flexible patterned film technology can be readily adapted to fabricate tissues with other complex geometries by changing the shape of the flexible film and custom mold, producing more biomimetic tissues for the study of development and disease.

### Biofabrication of Stem Cell-Derived, Anisotropic Cardiac Laminae for Modular Cardiac Tissue Engineering

The human heart has a complex 3D structure consisting of layered anisotropic myofiber sheets^{[1,2]}. The myofiber sheets are comprised of elongated, parallel cardiomyocytes, which are well aligned to the underlying extracellular matrix (ECM) fibers^{[3]}. In 3D, the orientation of these sheets in the ventricle changes gradually from a right-handed helix in the subendocardium to a left-handed helix in the subepicardium^{[4-6]}. This change in myofiber sheet orientation leads to a transmural helical structure in 3D^{[6]}. Studies have found that the complex, helical heart structure is critical to many aspects of adult heart function. Electrically, the depolarizing action potential is anisotropic, with the current guided by the fiber orientation in the heart^{[7-9]}. Mechanically, the fiber orientation is an important determinant of the myocardial stress and strain^{[10, 11]} and additionally affects the perfusion and oxygen consumption of the heart^{[12]}. Finally, the helical fiber orientation allows for the unique twisting contractile motion of the heart^{[13-15]}. This wringing motion is critical for appropriate blood clearance and cardiac output^{[16, 17]}. Additionally, altered cardiac tissue structure is often an indication of disease and can also contribute to deteriorating cardiac function in diseases such as hypertrophic cardiomyopathy and dilated cardiomyopathy, amongst others^{[18-20]}.

Induced pluripotent stem cell (iPSC) technology allows for the reprogramming of adult cells into pluripotent stem cells, which can then be differentiated into cardiomyocytes and other cardiac-specific cells. These cells can subsequently be used to engineer cardiac tissues for therapeutic, diagnostic or screening purposes, showing great promise in advancing medical treatments for cardiovascular diseases. However, current attempts to engineer cardiac tissue often fall well short of recapitulating this complex cardiac architecture and are often restricted to unidirectional^{[21-23]} or randomly organized 3D tissues^{[24, 25]}. Newer attempts to utilized decellularized whole hearts as a scaffold for seeded cardiomyocytes in a top-down approach show promise but still ultimately lack well-defined cardiac tissue structure and cell density^{[26, 27]}. This lack of physiologically structured, cell-dense cardiac tissues prevents optimal function due to the closely related structure-function relationship of the heart. This, in turn, limits the therapeutic and diagnostic efficacy of current engineered cardiac tissues. Alternatively, several groups have utilized a bottom-up approach to engineer cardiac tissues by manipulating surface cues, such as topography or patterned ECM proteins, to allow for the alignment of cardiomyocytes and cardiac monolayers^{[28-30]}. The advantage of a bottom-up approach allows for finer control of cell morphology and the ability to form well-aligned, cell-dense monolayers which more closely mimic the native myofiber sheets. However, one major limitation using these surface cues to engineer structured tissues is the inability to generate thick, anisotropic cell dense tissues characteristic of the myocardium.

Inspired by the underlying extracellular matrix of the myocardium, nanofabricated substrates were developed which allow for the robust and scalable alignment of single cardiomyocytes to anisotropic cardiac monolayers^{[3, 31]}. The successive stacking of these anisotropic cardiac sheets can yield aligned functional units of myocardium, similar to the 4 myocyte thick aligned myofiber sheets native to the heart, or even helically arranged multilayered cardiac tissues^{[10, 32-34]}. Such an approach should yield 3D, cell-dense cardiac tissues which can be engineered for a variety of purposes, such as aligned tissues which demonstrate improved contractile function due to the alignment of force vectors or helical tissues that more physiologically represent the transmural structure of the myocardium. To this end, the nanofabricated substrates were functionalized with a thermoresponsive polymer release layer, which would allow for the detachment of intact cell sheets by a reduction in culture temperature. However, cell sheets released by this method require cell-deposited extracellular matrix proteins to allow for the sheet to remain intact upon detachment. A stromal cell population of cardiac-specific endothelial cells was incorporated to allow for the deposition of ECM to detach intact, anisotropic cardiac cell sheets. Anisotropic cardiac cell sheets engineered using the TNFS are able to be transferred as viable individual monolayers or stacked together as 4-layered tissues. The stacked tissues can be engineered to be unidirectional or even helical in structure, which in turn affects contractile parameters such as contraction and relaxation velocity. These results demonstrate, for the first time, engineering of cell-dense cardiac tissues with precisely controllable structures, allowing for specific, reproducible and controllable investigations of the cardiac structure-function relationship.

Engineered 3D Human Cardiac Ventricular Models with Controllable Architecture for Studying Structure-Function Relationship

Tissue engineering combined with human induced pluripotent stem cell-derived cardiomyocytes (hiPSC-CMs) enables unique opportunities for creating physiological models the heart in the dish. However, there are few approaches available that can recapitulate the complex structure-function relationships that govern cardiac function at the macroscopic organ level. Here, scaled human 3D ventricular model with controllable cellular organization using patterned cardiac sheets is described. Surprisingly, spontaneous cellular remodeling was observed in the ventricular models pre-patterned with circumferential orientation, but not in those with other cellular organizations. Finite element model analysis found that cellular remodeling might occur to avoid high perpendicular shear forces by aligning parallel with them. Furthermore, anisotropic organization provides a functional benefit over isotropic organization when evaluated for their pumping function. This study provides an advanced platform for examination of human cardiac biomechanics and mechanobiology in a 3D physiological setting.

With every contraction, the heart exhibits a unique pumping function where the muscle fibers shorten, thicken in diameter, and elicit a twisting motion of the whole organ. Torsional movement is afforded by the distinctive double helix pattern of myofibrils throughout the thickness of the myocardium, where the orientation of muscle fibers exhibits a shift from a right-handed to a left-handed helix from the epicardium to the endocardium1. Relative to the short axis of the heart, the myofibers are orientated starting at −60° on the epicardial surface and shift to a +60° at the endocardial surface. The twisting motion of the heart is like the winding of a spring and is critical for suitable ejection of blood from the ventricles and therefore proper heart function. Subsequently, when the mechanics of this motion are disrupted by disease or injury, heart function is compromised. For example, myocardial disarray is associated with several forms of cardiomyopathy (e.g. dilated, hypertrophic, or infarction) and is often accompanied by structurally disruptive fibrotic scaring throughout the muscle.

In addition to the heart's distinct tissue architecture, it's morphogenesis from the early heart structures that resemble a tube in shape and fold into the adult four-chambered organ has been studied for over two centuries. Although animal models have provided a rich foundation of developmental biology from which can glean great insight of human development, there is still a dearth of knowledge surrounding how the helical myocardial tissue patterning is developed. This in part is likely due to a fundamental difference of species biology but also the difficulty of isolating and identifying biological governance over heart development in a complex whole-animal system. There is great evidence that mechanical cues may govern several aspects of heart morphogenesis. However, there are few models with which these findings can be substantiated in the context of human biology.

Tissue engineering strategies combined with human induced pluripotent stem cell (hiPSC) technology has enabled the development of diverse approaches for modeling structural and functional characteristics of cardiac tissue. These efforts have provided complementary platforms to animal models for modeling human cardiomyopathies and drug cardiotoxicity testing in the dish. However, most approaches yield two-dimensional (2D) laminar tissues or 3D structures that the lack structural complexity of the myocardium. There are few approaches that can recapitulate numerous aspects of the heart's multi-scale organization within a single platform has been difficult to incorporate cardiomyocyte anisotropy and the 3D geometry of the ventricles into existing models.

To address this gap of suitable technologies, a cell-sheet engineering approach utilizing flexible thermoresponsive nanofabricated substrates (fTNFS) was developed to enable production of 3D tissues with organized cellular architecture. In this study, this platform was adapted to engineer scaled, cardiac ventricular models with controllable architecture for study of the structure-function relationships within the myocardium. Three main structural organizations of the myocardial helix within the 3D tissue models were reviwed: circumferential (0°), angled (45°), and longitudinal (90°) cell orientations, and compare their contractile function to an isotropic control group with no cellular patterning. Anisotropic tissue architectures would outperform isotropic ones due to their alignment of forces produced during contraction.

Each of the tissue patterning schemes were possible with this approach and that the tissues were spontaneously active. Interestingly, a distinct remodeling event was observed in the circumferentially patterned group, where after four days in culture, the inner-most cell layers of the tissue reoriented perpendicular to their original orientation, while the outer layer becomes isotropic (randomly organized). These findings were modeled using finite element analysis. These simulations revealed a gradient of shear stresses and strains through the thickness of the tissue wall whose direction were perpendicular to the circumferential cellular organization. This result was not observed in simulations of any other groups. These large perpendicular shear forces developed during circumferential contraction may have provided a strong mechanical cue that promoted cells to remodel and align in its direction. Upon further functional evaluation by measuring pressure production, the remodeled and longitudinally patterned tissues were functionally superior to isotropic and angled tissues. Taken together, these results supported the hypothesis that 3D anisotropic tissue organization affords functional benefit and additionally creates complex patterns of mechanical cues that promote coordinated cellular remodeling. These findings have implications for how spatial patterns of mechanical forces present in 3D tissue microenvironments and might provide organizational cues during cardiac morphogenesis and development.

### General Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

As used herein, the term “hydrogel” is a type of “gel,” and refers to a water-swellable polymeric matrix, consisting of a three-dimensional network of macromolecules (e.g., hydrophilic polymers, hydrophobic polymers, blends thereof) held together by covalent or non-covalent crosslinks that can absorb a substantial amount of water (e.g., 50%, 60% 70%, 80%, 90%, 95%, 96%, 97%, 98%, 99% or greater than 99% per unit of non-water molecule) to form an elastic gel. The polymeric matrix may be formed of any suitable synthetic or naturally occurring polymer material. As used herein, the term “gel” refers to a solid three-dimensional network that spans the volume of a liquid medium and ensnares it through surface tension effects. This internal network structure may result from physical bonds (physical gels) or chemical bonds (chemical gels), as well as crystallites or other junctions that remain intact within the extending fluid. Virtually any fluid can be used as an extender including water (hydrogels), oil, and air (aerogel). Both by weight and volume, gels are mostly fluid in composition and thus exhibit densities similar to those of their constituent liquids. A hydrogel is a type of gel that uses water as a liquid medium.

The definitions of “hydrophobic” and “hydrophilic” polymers are based on the amount of water vapor absorbed by polymers at 100% relative humidity. According to this classification, hydrophobic polymers absorb only up to 1% water at 100% relative humidity (“rh”), while moderately hydrophilic polymers absorb 1-10% water, hydrophilic polymers are capable of absorbing more than 10% of water, and hygroscopic polymers absorb more than 20% of water. A “water-swellable” polymer is one that absorbs an amount of water greater than at least 50% of its own weight, upon immersion in an aqueous medium.

The term “crosslinked” herein refers to a composition containing intramolecular and/or intermolecular crosslinks, whether arising through covalent or noncovalent bonding, and may be direct or include a cross-linker. “Noncovalent” bonding includes both hydrogen bonding and electrostatic (ionic) bonding.

The term “polymer” includes linear and branched polymer structures, and also encompasses crosslinked polymers as well as copolymers (which may or may not be crosslinked), thus including block copolymers, alternating copolymers, random copolymers, and the like. Those compounds referred to herein as “oligomers” are polymers having a molecular weight below about 1000 Da, preferably below about 800 Da. Polymers and oligomers may be naturally occurring or obtained from synthetic sources.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

As used in the description herein and throughout the claims that follow, the meaning of “a,” “an,” and “the” includes plural reference unless the context clearly dictates otherwise.

### Kits Comprising the Tissue Engineering Scaffold

In aspects, a kit for producing the tissue engineering scaffold is provided. In embodiments, the kit comprises the tissue engineering scaffold and reagents.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention (e.g., a cardiovascular disease, neuronal disease, or a wound), one or more syringes, one or more applicators, or a sterile solution suitable for reconstituting a pharmaceutical composition of the present invention.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: Flexible TNFS Fabrication and Cell Sheet Stacking

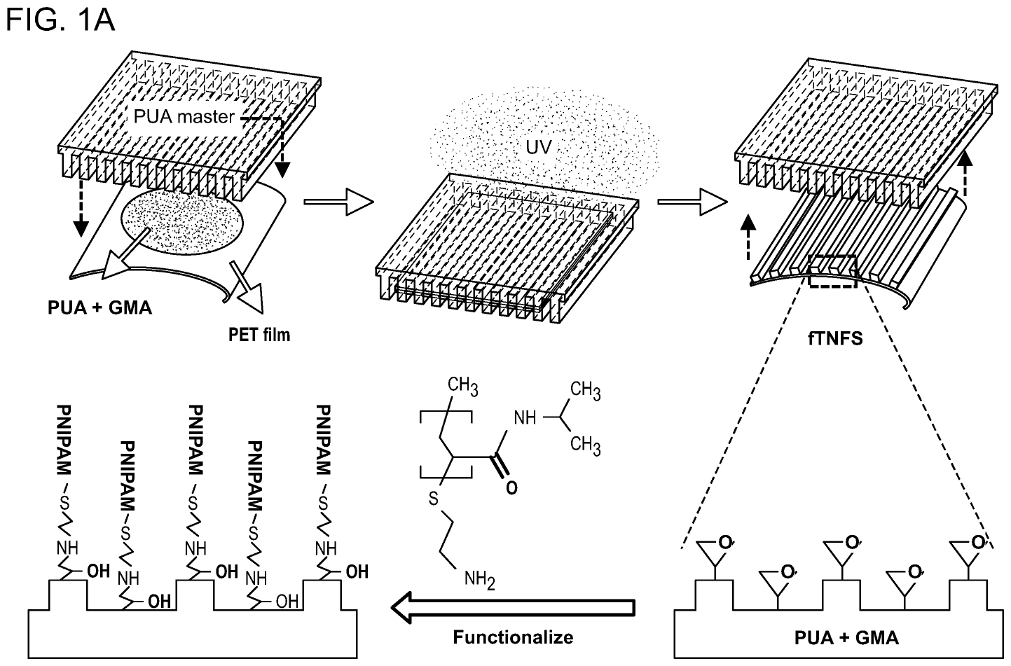

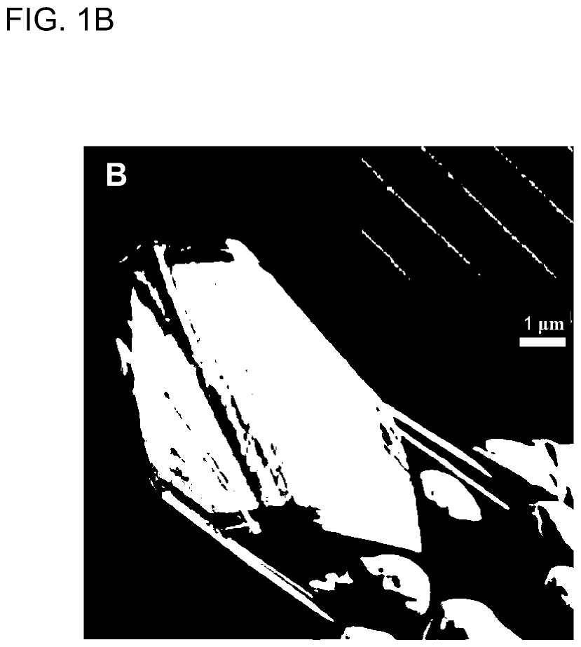

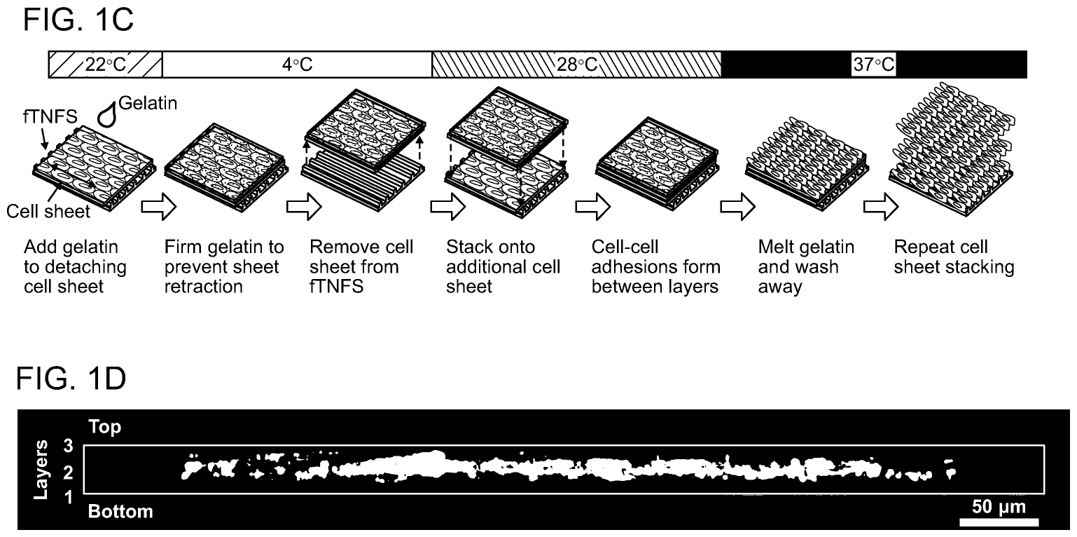

To develop a tissue engineering platform that would enable fabrication of 3D tissue geometries with control over local and global cellular patterning, The capillary force lithography techniques^{[16-18,28]} and amine-terminated poly (N-isopropylacrylamide) (pNIPAM)-mediated surface chemistry to produce flexible thermoresponsive nanofabricated substrates (fTNFS). Thermoresponsive functionalization was included to mediate the release of organized cell sheets from the nanopatterned surfaces without the use of digestive enzymes, such as trypsins, which are required to detach cells from traditional culture surfaces. Flexible films were chosen in this study to enable the fabrication of 3D tissues with curved surfaces by their capacity to be folded into a cylindrical shape. Large area flexible films (5 cm×5 cm) were patterned using a stiff polyurethane (PUA, 19.8 MPa) master mold with 800 nm ridges and groves and 600 nm depth (FIGS. 1A and 1B). When examined by scanning electron microscopy, fTNFS were found to have high pattern fidelity even after functionalization with a pNIPAM layer and bending with forceps (FIG. 1B, inset).

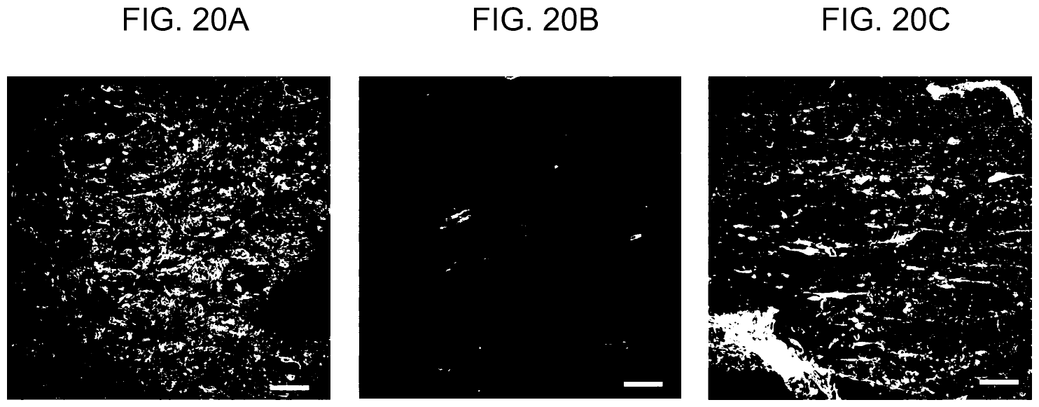

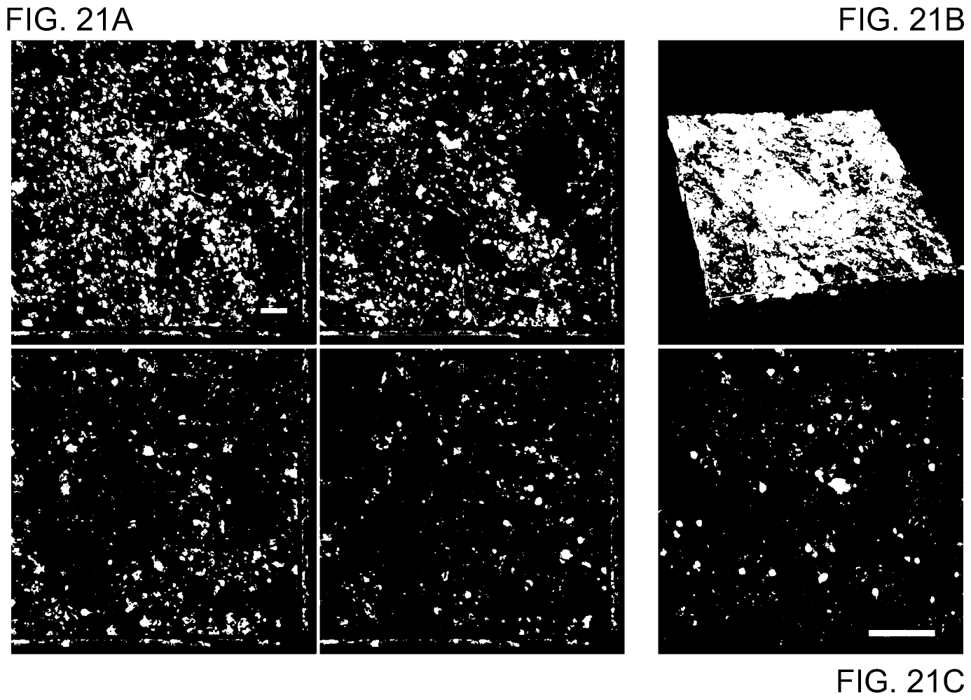

The speed and ease of cell-sheet detachment were optimized for each cell type by modulating the percentage of bound pNIPAM groups through increasing or decreasing the amount of glycidyl methacrylate (GMA) that is incorporated into the PUA layer during fTNFS fabrication^{[16,17]}.Smooth muscle cell (SMC) sheets required more pNIPAM-mediated release from the fTNFS using the gel-casting method (FIG. 1C) and 20% GMA was therefore blended into the PUA layer of the scaffolds. In contrast, skeletal myoblast (C2C12s) and cardiac sheets exhibited a tendency to spontaneously detach from fTNFS with higher GMA concentrations, and required much lower (1% GMA) levels of pNIPAM-mediated release. These differences in detachment may be due to the spontaneously contractile behavior of cardiomyocytes and the migration and fusion of muscle myoblasts during differentiation into myotubes. After optimization of GMA content, all cell sheet types could be detached and stacked to form multilayered tissues with maintenance of three discrete cell layers (FIG. 1D).

### Example 2: Fabrication of 3D Smooth Muscle Tissues

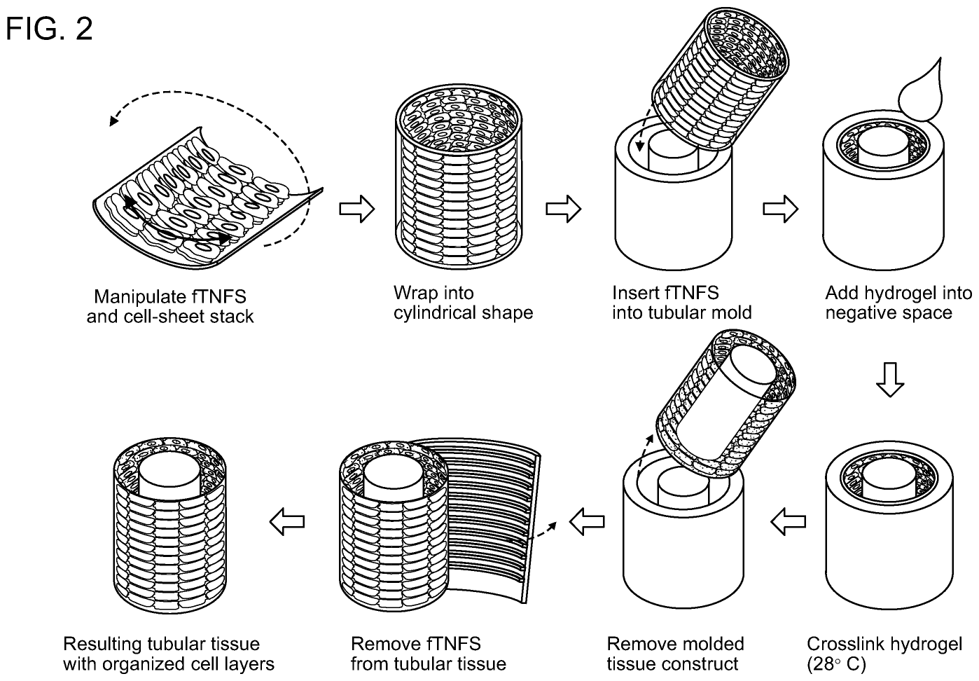

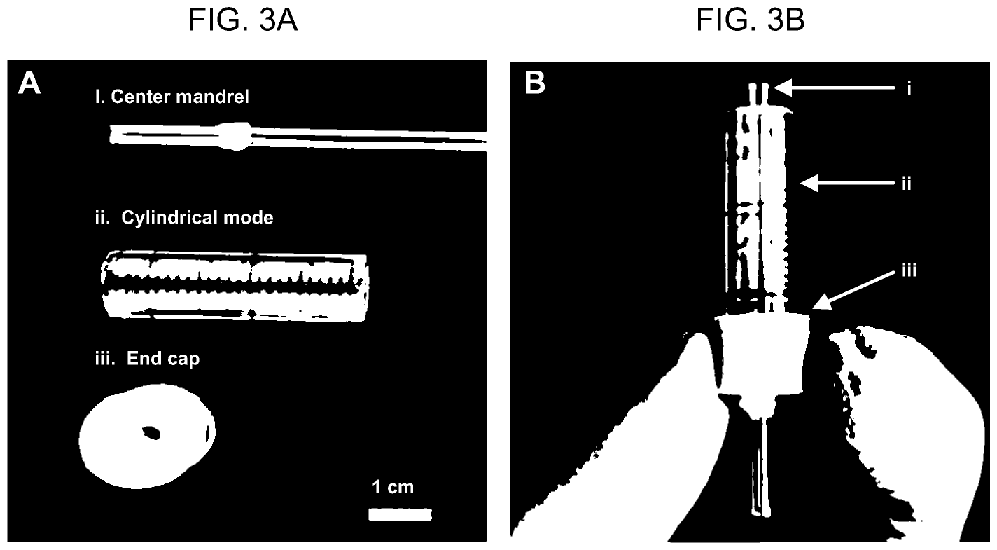

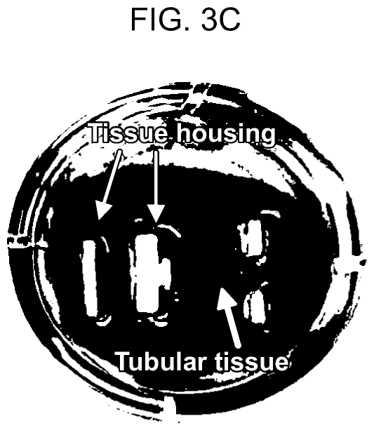

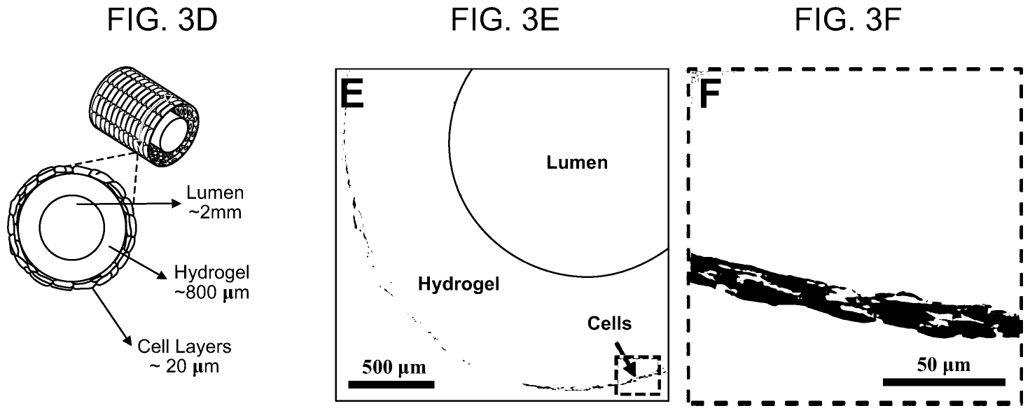

To demonstrate how the flexible nature of fTNFS allows for fabrication of 3D tissues with control over global cellular orientation, a simplified tubular structure was modeled to mimic the geometry of vascular structures. In blood vessels, vascular tone and blood flow are regulated by SMC contraction and relaxation. Smooth muscle cells make up the medial layer of blood vessels, the tunica media, and are organized in a circumferential pattern[^{1,29]}. To recapitulate architecture of the tunica media, the fTNFS was patterned such that the nanogrooves and ridges were parallel to the long axis of the rectangular scaffold. To form a cylinder with circumferentially layered SMC-sheets, the fTNFS were then rolled along the short axis with the cell layers on the inside of the lumen (FIG. 2). This cylinder was then inserted into cylindrical mold with a capped end and center mandrel (FIGS. 3A and 3B). The void space between the mandrel and the SMC-sheet cylinder was filled with a crosslinking gelatin hydrogel to provide a structured tubular shape of the final tissue. Finally, the SMC-sheet cylinder and crosslinked hydrogel were removed from the mold followed by the unwrapping of the fTNFS. The resulting tubular tissue possessed a hollow lumen (diameter=2.0±0.9 mm) surrounded by hydrogel walls (thickness ˜800 μm) (FIGS. 3C and 3D). The cell layers were wrapped around the outer edge of the hydrogel tube (cell layer thickness=17±3 μm) and were not encased by the hydrogel during the casting process (FIG. 3E). The tissue was then gently manipulated with forceps onto a custom tissue housing for culture and visualization with an inverted microscope (FIG. 3C). After 7 days in culture, SMC tubes were cross-sectioned and histologically stained. Three distinct cell layers were maintained around the outer edge of the hydrogel walls with the center lumen still intact (FIG. 3F). Furthermore, the cell bodies and their nuclei had maintained circumferential alignment and elongation along the hydrogel's edge after several days in culture.

### Example 3: Fabrication of 3D Cardiac and Skeletal Muscle Tissues

The muscle structures throughout the body have multiple stratified layers of organized cells and varying curved 3D geometries. For example, limb muscles have a spindled shape with tapered ends, while trunk muscles, such as the transvers abdominis and oblique muscles, are curved around the side of the body. Furthermore, cardiovascular and digestive organs possess hollow lumens with layers of organized muscle, such as the stomach, intestines, and the chambers of the heart.

There have been several approaches to modeling these tissue organizations in vitro, such as seeding engineered scaffolds^{[30-32]}, 3D bioprinting^{[13,33]}, cell sheet layering^{[26,34-37]}, and tissue casting^{[38-41]}. However, few of these approaches can recapitulate the anisotropic layering of organized cell-sheets that ultimately gives rise to tissue functionality. To address this limitation, the fTNFS technology and tissue casting process was applied to fabricating organized multilayered skeletal and cardiac muscle tubes with curved surfaces. The fTNFS were seeded with either mouse skeletal muscle myoblasts (C2C12 cells) or hiPSC-derived cardiomyocytes and endothelial cells to form organized monolayers. Endothelial cells were included in cardiac monolayers as a stromal cell component which improves the integrity of formed and detached cell sheets through a combination of increased intercellular coupling and additional ECM deposition. In contrast, monolayers of cardiomyocytes alone did not maintain a contiguous cellsheet during detachment, but rather individual cells pulled away from one another, resulting in the detachment of small clusters (Supplemental FIG. 1). Skeletal and cardiac monolayers were detached and stacked to create multilayered constructs using the gelcasting process and subsequently cast into tubular geometries as described above.

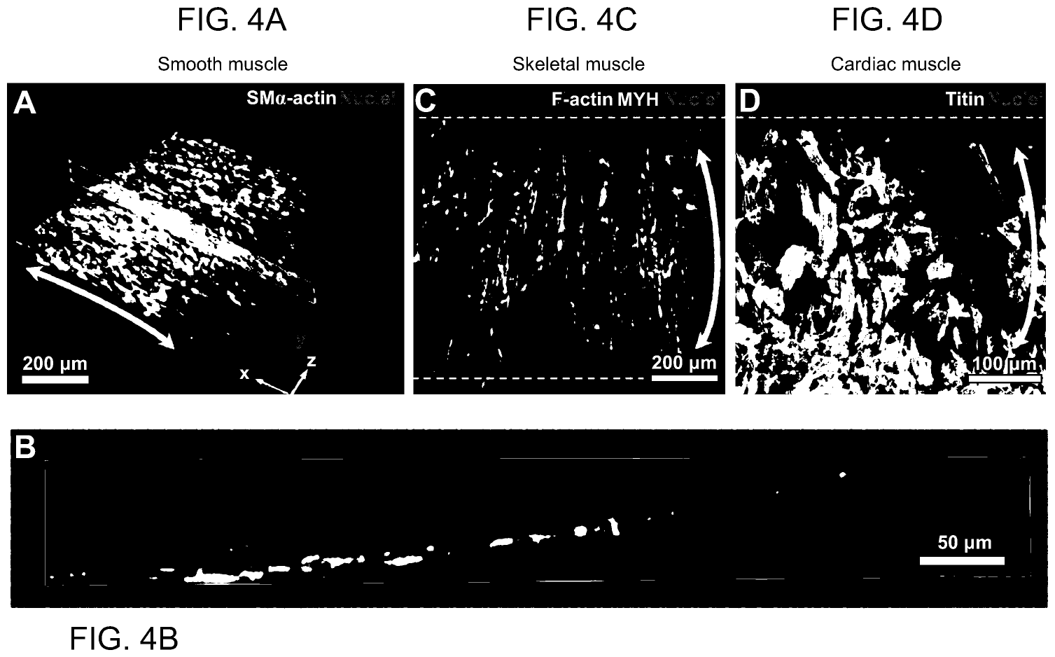

Upon removal from the casting mold and unwrapping of the fTNFS, skeletal and cardiac tubes were found to have global cell coverage on the curved outer edges of the tissues and possessed hollow lumens, similar to the SMC tubes (FIGS. 4A-4E). Cardiac tubes began coordinated, spontaneous contractions after 1-2 days in culture, demonstrating that the cell-cell connections had been maintained within the cardiac sheets after the casting process. In the case of skeletal muscle tubes, tissues were cultured in a serum-rich (20% FBS) medium for 3-4 days after fabrication to promote additional cell growth before switching into a serum-poor (2% HS) medium. Once in low serum conditions, myoblasts began to fuse into multinucleated myotubes that elongated circumferentially around the tube's curved surface (FIG. 4C). This result suggests that pre-patterning individual myoblast cell sheets before incorporation into 3D tissues is sufficient to provide robust organizational cues from within the cellsheet's structure and does not require sustained external cues to generate aligned myotubes. The deposited ECM during cell-sheet formation was also organized and provided robust directional cues that promote consistent cellular alignment after casting into a 3D tissue.

Sheets of aligned C2C12 myoblasts were transferred onto another sheet with either parallel or orthogonal alignment^{[17]}. The alignment of the deposited ECM within each sheet while cultured on fTNFS was maintained after stacking and promoted the formation of parallel or orthogonally organized myotubes within each layer, respectively. Furthermore, sheets of myoblasts stacked in parallel alignment were found to have longer myotubes and higher fusion indices compared to sheets stacked in an orthogonal orientation^{[17]}. Pervious results^{[17]} taken together with those described in this study demonstrate the significant influence that the ECM has on tissue development and structure.

### Example 4: Cellular Organization was Maintained in 3D Tubular Tissues

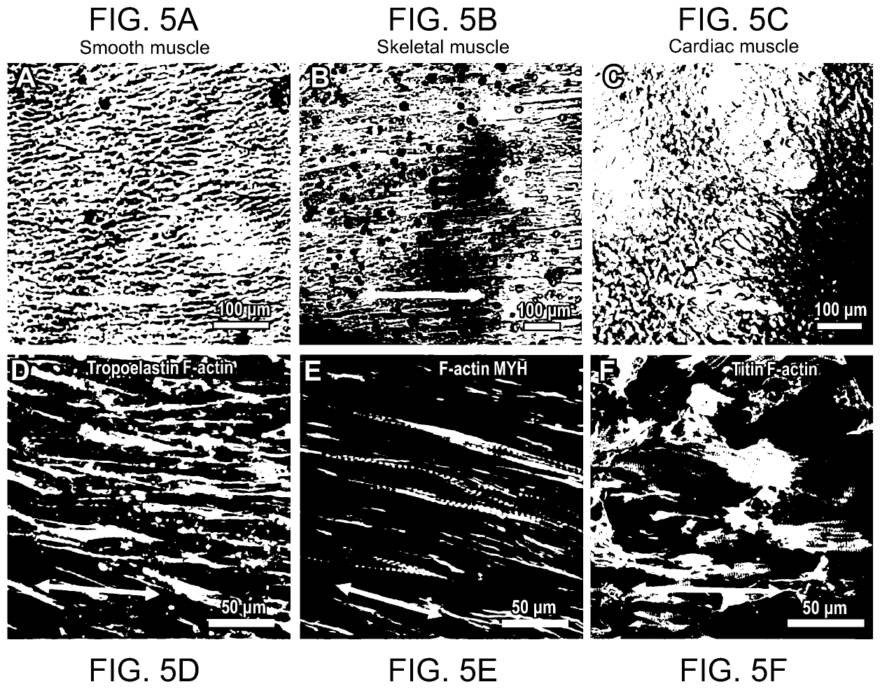

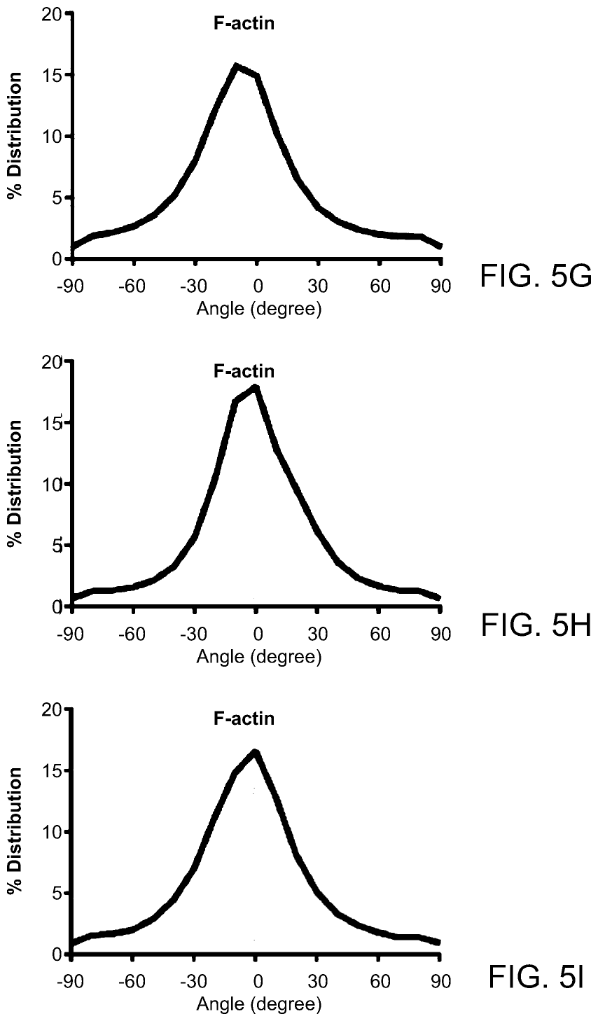

To investigate if circumferential patterning of cellular alignment was maintained over longer culture periods, smooth, skeletal, and cardiac muscle tubes were cultured for 7 or 14 days. Each engineered tissue was stained for filamentous actin (F-actin) and its organization was quantified using alignment analysis MATLAB scripts as described previously (FIG. 5A)^{[16,27]}. Smooth, skeletal, and cardiac muscle tubes demonstrated similar levels of circumferential cellular alignment around the tubes' surfaces (FIGS. 5D-5I).

Skeletal muscle tubes showed formation of elongated circumferential myotubes after 3-4 days in culture with medium containing low-serum, which promotes fusion and differentiation of myoblasts^{[33,42,43]}. Myosin heavy chain (MYH, all isoforms) was expressed throughout fused myotubes at earlier timepoints (FIG. 4C). However, with application of broad-field electrical stimulation (1 Hz, 10 V, 24 ms pulses) no myotube contraction was observed suggesting contractile proteins had not yet been assembled into functional sarcomeres. To promote the formation of functional sarcomeres, chronic broad field stimulation was applied at lower voltages (1 Hz, 3 V, 24 ms) 3-5 days after myotube fusion was apparent in differentiation medium conditions, as shown by others^{[44,45]}. As early as 2-3 days after application of chronic electrical stimulation, myotube twitching followed by robust contraction was visualized in sync with the 1 Hz stimulation pacing and halted in the absence of an electrical pulse. After 9 days of chronic electrical stimulation, registered sarcomeres were easily detectable within myotubes when visualized with immunocytochemistry (FIG. 5E). Other groups have also substantiated the role of electrical 5I) stimulation in the formation of functional skeletal myotubes in vitro and have demonstrated that sodium and calcium flux, through voltage-gated ion channels, may be required for Z- and A-band formation^{[44-47]}. Together, these data support observations made in this study and suggest that incorporation of both internal and external developmental cues may be required in tissue engineering approaches to recapitulate in vivo-like environments and to promote functional maturation.

Cardiac tubes were also subjected to chronic stimulation pulses (1 Hz, 3 V, 8 ms) for up to 37 days in culture. After 37 days in stimulated culture, cross-sectional videos of cardiac tubes contracting under broad-field electrical stimulation showed that the hydrogel walls could be deformed during contraction. This result demonstrated that patterning and layering aligned cardiomyocytes onto curved three-dimensional tissues was possible and that their contractile function was maintained in long-term culture. In future applications of this technology, long-term electrical stimulation protocols with increasingly challenging pacing frequencies could be applied to promote maturation of cardiac tubes as shown by other groups^{[6,48,49]}. It would be interesting to explore if pre-patterning of cardiomyocyte architecture within 3D ventricular models would enhance or accelerate maturation when combined with electrical and or mechanical conditioning. Additionally, this technique provides a novel approach for recapitulating more complex myocardial architectures. For example, in the myocardium of the left ventricle, every four to five layers of cardiomyocytes (or myolaminae) are aligned in a single plane but the alignment direction of each myolamina shifts by approximately 10°.

This allows the myocardium to encompass a helical fiber architecture with a 180° range of orientations and efficiently maximize its ejection fraction of blood from the ventricles with each contraction of the heart^{[50-52]}. By patterning and stacking individual sheets of cardiomyocytes, this approach could be used to model microenvironments that cardiomyocytes experience at cleavage planes during contraction. Furthermore, if wrapped into a 3D ventricle shape, this platform could be used to study how varied cardiac tissue organizations contribute to ventricle-level function.

### Conclusions

In this study, a novel method for patterning and layering individual cell sheets and casting them into 3D tubular geometries with curved surfaces. Custom molds were used to cast tubular tissues inspired by the vasculature and the curved tissue structures of the heart ventricles and skeletal muscles in the body's trunk. Pre-patterning individual cell sheets promoted cellular alignment in 3D tissues for several weeks after tissue casting. In addition to providing tissue-level alignment cues, broad-field electrical stimulation for skeletal tubes and found that electrical conditioning was required to promote contractile function. These results suggest that a combination of internal and external conditioning cues may be required to further mature tissues fabricated using fTNFS-enabled cell-sheet casting.

Given the versatile nature of the fTNFS platform, this approach be adapted to fabricate uniquely shaped flexible films and tissue-specific shaped molds for even more complex tissue architectures, such as the conical ventricles of the heart. In this study, tissues were created with thicknesses of 3-4 cell layers. However, thicker tissues could be generated that surpass the limits of nutrient and oxygen diffusion and prevent tissue necrosis, by incorporating vascular networks or proangiogenic factors^{[53]}. Providing vascular networks could enable longterm culture of thicker tissues for maturation studies. Additionally, this system could be further adapted by incorporating biochemically tunable hydrogels (e.g. fibrin, photo-crosslinking gels, decellularized-ECM, etc.) for tissue specific customization and or presentation of embedded signaling factors for developmental and maturation studies. Flexible TNFS could enable the fabrication of more advanced engineered tissues that could be used to investigate complex structure-function relationships, development, and maturation in the dish.

### Materials and Methods

Fabrication of Flexible Thermoresponsive Nanofabricated Substrates (fTNFS)

To fabricate flexible films with nanotopographical cues and thermoresponsive properties, capillary force lithography was utilized^{[6-18]}. Briefly, nanopatterned films were fabricated using 100 μL of a polymer curable by ultraviolet light (UV), polyurethane acrylate (PUA, Norland Optical Adhesive #76) mixed with either 1% or 20% (w/w) glycidyl methacrylate (GMA). The UV-curable polymer was sandwiched and spread between a 23 pm-thick flexible poly-ethylene terephthalate (PET) film and a PUA master mold with parallel ridges and grooves that were 800 nm in width and 600 nm in depth (FIG. 1A). The mold and film construct were exposed to high intensity 365 nm wavelength UV light for 1 min to polymerize the PUA-GMA solution. After initial polymerization of the sandwiched polymer layer, the flexible film and adhered nanopatterned polymer layer were carefully removed from the master mold using forceps (FIG. 1B). The newly constructed nanopatterned film was placed under low intensity 365 nm UV light for 24 h to ensure complete polymerization of the PUA-PGMA polymer. To provide thermoresponsive functionality, nanopatterned substrates were then dip-coated with an amine-terminated poly (N-isopropylacrylamide) (pNIPAM) solution (13 μM in H2O, Mn=2500 Sigma-Aldrich) for 24 h on a tabletop rocker (55 rpm, room temperature). After 24 h, excess pNIPAM was removed from the flexible thermoresponsive nanofabricated substrates (fTNFS) through three 5-min washes with deionized water and allowed to dry overnight. The films were cut into rectangular sheets (1.25 cm×1.5 cm) using a die cutter. The fTNFS were exposed to 294 nm UV light for 1 h in a biosafety cabinet for sterilization prior to use.

In order to restrict cell-seeding to the fTNFS surface and minimize cell waste, two fTNFS were temporarily affixed into the bottoms of custom polydimethylsiloxane (PDMS, Sylgard 181) rectangular wells (13.5 mm×30 mm) using porcine gelatin (7.5% w/v, Sigma) crosslinked with transglutaminase (MooGloo™ TI-TG, Modernist Pantry) as an adhesive. Flexible TNFS were incubated with fetal bovine serum (FBS, Sigma) overnight at 4° C. before cell seeding to deposit a thin protein layer to promote cell adhesion to the surface.

Scanning Electron Microscopy of fTNFS

Poly-NIPAM-functionalized fTNFS were sputter-coated with Au/Pd alloy prior to imaging using scanning electron microscopy (Sirion XL30, FEI, OR, USA). Images were taken with an acceleration voltage of 5 kV and spot size of 2.

Cell Culture and Cell-Seeding of fTNFS

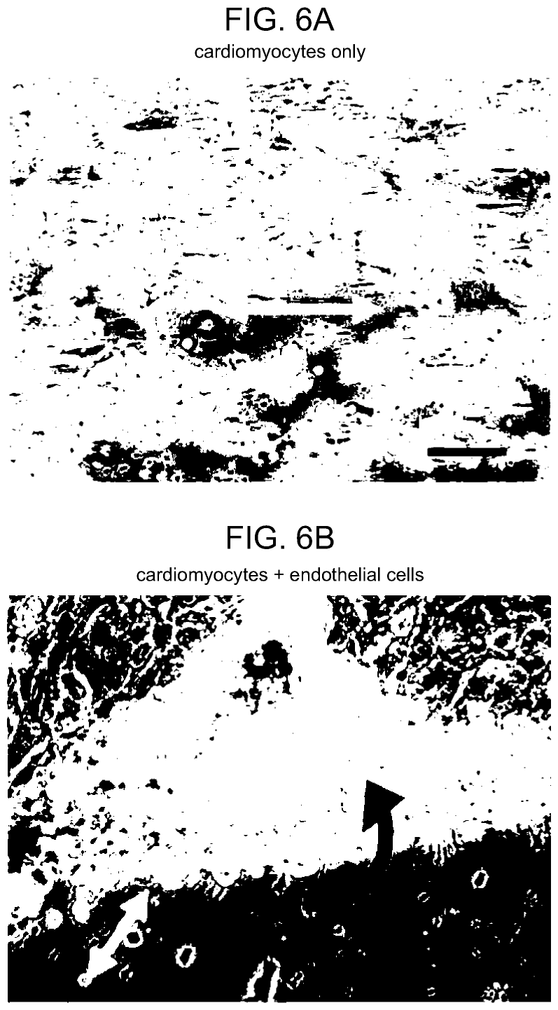

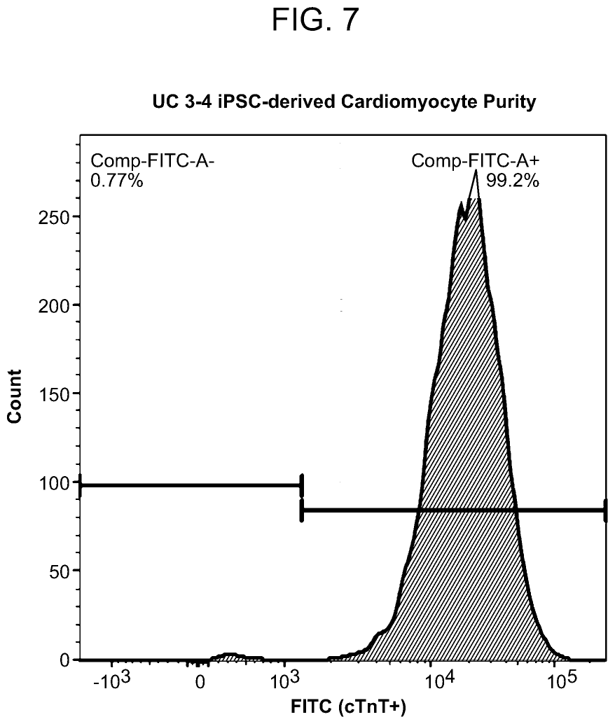

Mouse SMCs were cultured on tissue-culture treated plastic dishes with Dulbecco's Modified Eagle's Medium (DMEM, Gibco) supplemented with 1% penicillin-streptomycin (p/s, Sigma), 10% FBS. Cells were passaged at 80% confluency during expansion and only passages 30 and below were used to minimize confounding effects of cell senescence on tissue fabrication. Cells were split and seeded onto fTNFS at a density of 175,000 cells/cm2 in 1 mL of medium and allowed to adhere overnight at 37° C. and 5% CO₂. Seeded cells were cultured for 5-7 days before cell sheet stacking and tissue fabrication to allow a highly confluent monolayer of cells to form. Observation of cell growth was conducted using a bright-field microscope (Nikon TS100). C2C12 mouse muscle myoblasts (C2C12s; ATCC) were cultured under the same conditions as the SMCs as described above and seeded at 175,000 cells/cm2 onto fTNFS. However, seeded cells were cultured for 2-3 days before cell sheet stacking and tissue fabrication as C2C12 cells were found to proliferate at a faster rate than SMCs. Three to four days after tissue fabrication, tissue constructs were cultured in a low-serum containing medium (DMEM, 2% horse serum (HS), 1% p/s) to promote fusion and differentiation of myoblasts into myotubes. To promote further functional and structural maturation of myotubes, chronic broad-field electrical stimulation was applied (1 Hz, 3 V, 24 ms; IonOptix C-Pace) after 3-4 days of culturing tissues with low-serum differentiating medium and once myotube formation was observed over the entire tissue surface area. Cardiomyocytes (CMs) and endocardial-like endothelial cells (ECs) were differentiated from human induced pluripotent stem cells (hiPSCs, UC 3-4) derived from patient urine samples^{[20]}. Endothelial cells were included to promote cell sheet formation as monolayers of pure cardiomyocytes were found to clump during tissue fabrication (FIGS. 6A-6B). Established monolayer-based differentiation protocols were used that modulate Wnt-signaling pathways with small molecules to specify cardiac mesoderm lineages^{[}21,22]. In brief, UC 3-4 hiPSC colonies were maintained on Matrigel (1:60, Corning) coated tissue-culture plates in mTeSR medium until 80% confluency. Colonies were then replated into a monolayer at 250,000 cells/cm2 (high density) or 100,000 cells/cm2 (low density) for directed differentiation of cardiomyocytes (CMs) or endothelial cells (ECs), respectively^{[22,23]}. To drive differentiation toward the cardiomyocyte lineage, high density monolayers were exposed to CHIR-99021 (10 μM, Fischer Technologies) in Roswell Park Memorial Institute 1640 (RPMI) medium with B27 without insulin (Gibco) on day 0 (induction) to activate the Wnt signaling pathway and specify mesoderm gene expression. High-density monolayers were exposed to the Wnt-inhibitor IWP4 (4 μM, Stemgent) on day 3 to further specify cardiac mesoderm and were then cultured in RPMI-B27 medium with insulin from day 7 and onward.

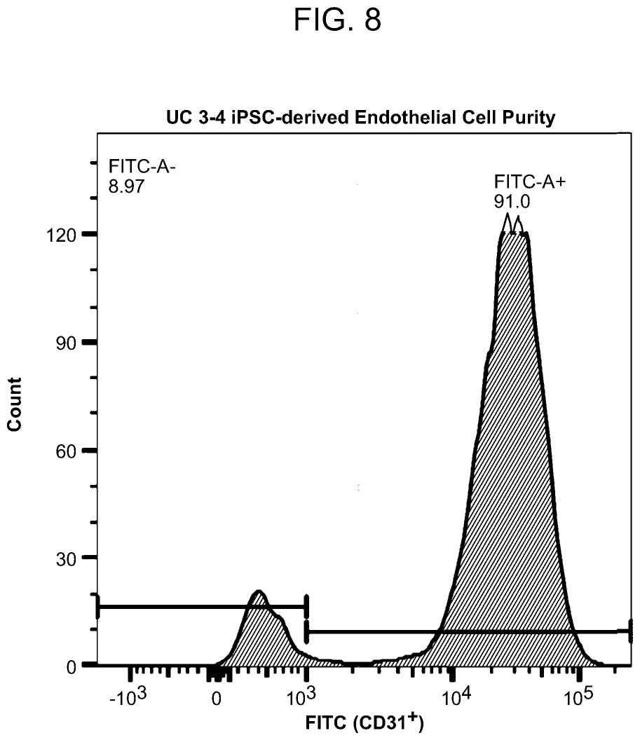

Beating monolayers of cardiomyocytes were observed as early as day 9. Cardiac-differentiated populations were then subjected to a lactate-rich, glucose-poor selection medium at day 14 for 3 days to enrich the cardiomyocyte population^{[24]}. Cells were harvested on day 17 and fixed in 4% paraformaldehyde as a single-cell solution and prepared for flow cytometry to determine cardiomyocyte purity. Cells were stained with a mouse-anti-cardiac troponin T (cTnT) antibody (1:100, Thermo-Scientific) and counterstained with a goat-anti-mouse Alexa Fluor 488-counjugated antibody (1:200, Invitrogen). Cell populations used for this study were at least 95% cTnT-positive when analyzed by flow cytometry (FIG. 7). Similarly, to drive differentiation towards the cardiac endothelial lineage, low density monolayers were exposed to activin-A (100 ng/mL, R&D Systems) and Matrigel in RPMI with B27 on day 0 (induction). On day 1 post-induction, low-density monolayers were exposed to bone morphogenic protein-4 (BMP-4; 5 ng/mL, R&D Systems) and CHIR-99021 (1 μM) in RPMI-B27 medium. To specify the endothelial lineage, low-density monolayers were switched into StemPro-34 medium, containing vascular endothelial growth factor (VEGF; 300 ng/mL, PeproTech), BMP-4 (10 ng/mL), basic fibroblast growth factor (bFGF; 5 ng/mL, R&D Systems), ascorbic acid (50 μg/mL), and monothioglycerol (4 μm). On day 5, the low-density monolayers were re-plated at 13 k/cm2 into gelatin-coated tissue culture plates and expanded in Endothelial Growth Medium-2 (EGM-2, Lonza) supplemented with VEGF, bFGF, and CHIR-99021 (1 μM) until day 11. Live cells were stained with a mouse-anti-CD31 antibody pre-conjugated with an Alexa Fluor 488 fluorophore (1:100, R&D Systems) for 1 h on ice and flow cytometry was performed immediately. All EC populations used in this study were at least 90% CD31-positive when analyzed by live-cell flow cytometry on day 11 (FIG. 8). ECs were then seeded with cardiomyocytes immediately or cryopreserved for later use. To limit possible confounding factors associated with age variation in the CM or EC population, CMs were used within 17-30 days postinduction and ECs used between 12 and 15 days post-induction for tissue fabrication. Purified CMs and ECs were seeded onto fTNFS such that the final proportion of CMs and ECs was 88% and 12% (˜7:1 CMs:ECs) of the total cell number, respectively. This CM:EC ratio was optimized during preliminary experiments to yield highly aligned and confluent cell sheets that could withstand previously published cellsheet detachment and stacking process^{[16,17]}. Cardiac cell sheets were cultured for 10-14 days in cardiac growth medium (64% RPMIB27+insulin, 25% EGM-2, 10% FBS, 1% p/s) before cell sheet stacking and tissue fabrication to allow for the formation of highly dense and confluent monolayers.

### Cell Sheet Stacking

Cell sheets that form dense monolayers through cell-cell connection and ECM deposition exhibit a tendency to clump or fold inward on themselves once detached from cell culture surfaces. To prevent this, a gel casting method for stacking aligned cell sheets that conferred layer-by-layer control over tissue architecture (FIG. 1C)^{[16,17]} was developed. Briefly, patterned cell sheets were detached from the fTNFS by lowering the culture temperature to 22° C. (room temperature), below the lower critical solution temperature point of pNIPAM (32° C.), where the hydrophobicity of pNIPAM abruptly and dramatically switches to a hydrophilic hydrogel. This hydrogel polymer then swells and dislodges intact cell sheets^{[25,26]}. After incubation for 30 min (for C2C12 and cardiac sheets) or 1 h (SMC sheets) at room temperature, and just before complete detachment, cell sheets were cast in a 7.5% w/v gelatin solution at 4° C. for 30 min to maintain the anisotropic organization of the cell sheet and prevent sheet retraction. The gel-casted cell sheets were moved into a 28° C. incubator for 1 h to further promote cell detachment from the fTNFS without melting the gelatin that maintains the cellular alignment. The gel-casted sheets were then incubated at 4° C. for 15 min to allow the gelatin to further solidify for better handleability. The gel-casted cell sheet was then removed from the fTNFS with forceps and stacked on top of another cell sheet with parallel cellular orientation to produce an aligned, bi-layered laminar tissue. Once stacking was complete, the gelatin was completely dissolved 37° C. and the construct washed with warm (37° C.) medium to ensure the remaining tissue structure constitutes a scaffold-free, bilayered cell sheet construct on top of a fTNFS. This process was then repeated to add a third and final cell layer with parallel orientation. To visualize maintenance of three discrete cell layers, each cell sheet was labeled prior to stacking by incubating with either a red or green cell dye (2 μM CellTracker CMFDA Green or 2 μM CellTracker Red CMTPX, Invitrogen) for 30 min. Z-stacks were then taken with a confocal microscope (Nikon A1R, 10× objective) to visualize the alternately layered red, green, then red cell sheets (FIG. 1D).

### Flexible TNFS Manipulation and 3D Tissue Casting

After stacking three-layered tissues as described above (FIG. 1C), trilayered tissue constructs were then cast into a 3D tubular geometry using polystyrene cylindrical molds and custom 3D-printed casting implements (FIGS. 2 and 3A-3D). The tri-layered tissue and fTNFS were first incubated in room temperature phosphate buffered saline (PBS, Gibco) for 30 min to promote the basement layer to detach from the fTNFS. Simultaneously, the polystyrene center mandrel (FIG. 3A. i.), cylindrical mold (FIG. 3A. ii.), and 3D-printed end cap (FIG. 3A. iii.) were coated with a pluronic F-127 solution (5%, Sigma) solution to prevent the final tissue construct from sticking upon removal from the mold. The fTNFS with cell sheets were manipulated with forceps into a cylindrical shape with the cell layers facing inward and inserted into the cylindrical mold (FIGS. 2 & 3A. ii.). The end cap (FIG. 3A. iii.) was then placed on the end of the cylindrical mold and the center mandrel (FIG. 3A. i.) was inserted into the assembly through the hole in the end cap. The final casting assembly (FIG. 3B) ensured that the lumen created in the tissue construct was straight and the resulting tissue walls were of uniform thickness on all sides. The remaining negative space within the casting tube was then filled with 200 μL of warmed gelatin and transglutaminase crosslinker (10% TG in PBS, MooGloo™ TI-TG; 10% porcine gelatin w/v in DMEM, Sigma) and allowed to crosslink at 28° C. for 1 h; the final concentration of crosslinked gelatin was 5%. After the gelatin was crosslinked, the molded tissue constructs were incubated at 4° C. for 30 min to allow the basement layer of cells connected to the fTNFS to detach. The fTNFS and tubular tissue with cells was then carefully removed from the casting assembly with forceps and the fTNFS was unwrapped from the tubular tissue. The final tubular tissue was attached to a custom 3D printed tissue housing (FIG. 3C).

### Tissue Histology and Dimensional Measurements

To visualize cellular orientation and quantify tissue dimensions after 7 days in culture, SMC tubular tissues were fixed in paraformaldehyde (PFA; 4% in PBS) for 30 min at room temperature and washed with PBS. Tissues were then dehydrated by serial washes in ethanol (50%, 60%, and 70%) for 20 min before embedding in paraffin blocks for sectioning. Tubular tissues were cut using a cryostat (Leica CM1950) to make 4 μm cross-sections along the short access of the tube allowing for visualization of the center lumen, tissue wall thickness, and cell layer thickness (FIGS. 3E and 3F). Sections were stained with hematoxylin and eosin to visualize extracellular matrix and cytoplasm (pink) and DNA (blue). ImageJ (National Institutes of Health) measurement tools were used to measure tissue wall thickness, cell layer thickness, lumen diameter, and outer diameter across 30 sections. Measurements of each dimension were averaged and standard deviation was calculated (GraphPad, Prism).

### Immunostaining and Confocal Imaging

Tubular tissues were cultured for 7 days after casting and fixed in paraformaldehyde (PFA; 4% in PBS) for 30 min at room temperature and washed with PBS. To visualize intercellular proteins, tissues were permeabilized in 0.2% Triton-X 100, 0.5% BSA, and 5% goat serum in PBS at room temperature for 1 h and transferred into a blocking solution of 5% goat serum with 0.5% BSA in PBS for 2 h to prevent nonspecific antibody binding. Primary antibodies (mouse-anti-smooth muscle α-actin (1:200, SMα-actin, Abcam), mouse-anti-myosin heavy chain (1:50, MYH, A4.1025 Developmental Studies Hybridoma Bank, The University of Iowa, Department of Biology; deposited by the Baxter Lab for Stem Cell Biology at Stanford University), rabbit-anti-titin (1:300, Myomedix) were diluted in a staining solution of 1.5% goat serum in PBS and incubated with the respective tissues overnight at 4° C. Excess primary antibodies were washed away through three 5-min washes with PBS before corresponding secondary antibodies (1:400, Alexa 488, 594, or 647, Invitrogen) and conjugated phalloidin (1:200, F-actin, 488 or 647, Invitrogen) were applied in 0.5% BSA in PBS for 2 h at room temperature. Excess secondary antibodies were washed away through three 5-min washes with PBS before a nuclear counterstain (DAPI, Invitrogen) was applied.

Given the relatively large 3D geometry of the tubular tissues, custom mounting chambers were developed by placing a square 3 mm thick PDMS frame around the tissue and sandwiching them between two rectangular cover-glasses (0.17 mm thickness, Fisher Scientific). The tissues were stored in anti-fade mounting medium (VECTASHIELD, Vector Laboratories) within the PDMS mounting chambers. The rounded surfaces of the tubular tissues were slightly flattened to visualize their cellular layers with a confocal microscope, but the overall curvature of the tissue was maintained. Confocal z-stacks were taken of tubular tissues using either a Nikon A1R and or a Yokogawa W1 spinning-disk confocal microscope, and 10×-dry, 20×-dry, or 40× oil-immersion objectives.

### Cellular Orientation Analysis

To quantify cellular orientation in 3D tubular tissues, confocal images of cytoskeletal filamentous actin (F-actin) for SMC, C2C12, and cardiac tubes were taken of three different areas using a 40× oil-immersion objective. These images were analyzed using a modified MATLAB script (MathWorks) that utilizes pixel gradient analysis to determine the distribution of orientation angles within an image[^{16,27]}. Briefly, a Gaussian low pass filter and Sobel horizontal edgeemphasize filter are applied (as predefined by the MATLAB Image Analysis Toolbox) to create a 2D convolution. The Sobel filter is then transposed to extract horizontal and vertical edges and then used to calculate the gradient magnitude of each pixel within the image. The images were then thresholded to define the edges of single cells and calculate their orientation angles relative to the x-axis at 0°. These orientation angles were then binned and plotted as histograms to represent the overall cell alignment of the 3D tissue (FIGS. 5G-I).

### Example 5: Formation of Anisotropic Cardiac Cell Sheets on the TNFS Requires Consistent Input Cardiomyocytes and Specific Surface Chemistry

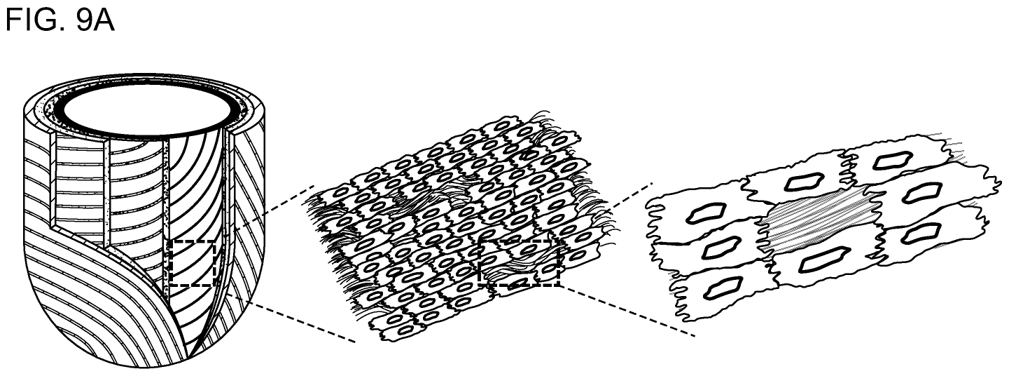

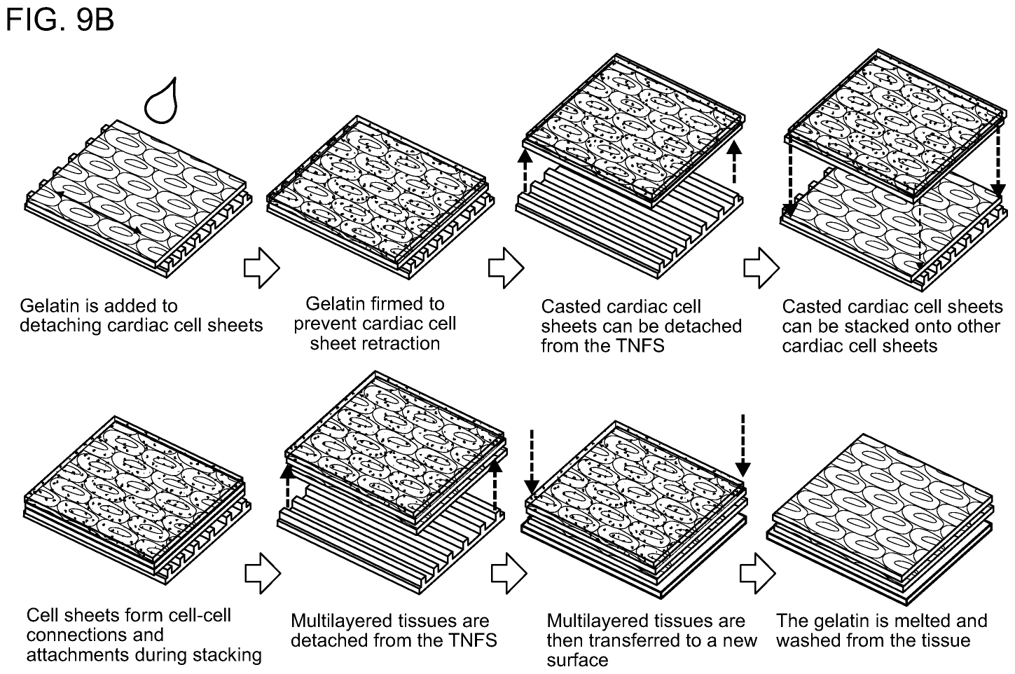

To engineer 3D, anisotropic cardiac tissues, bioinspired nanotopographical cues were used mimicking the aligned, native cardiac ECM fibers found in the myocardium (FIG. 9A). Although differentiation of hiPSCs into cardiomyocytes can yield highly pure cardiomyocyte populations, there are often variabilities between differentiation runs as well as a small percentage of non-cardiomyocytes which can increase variability of subsequent experiments.

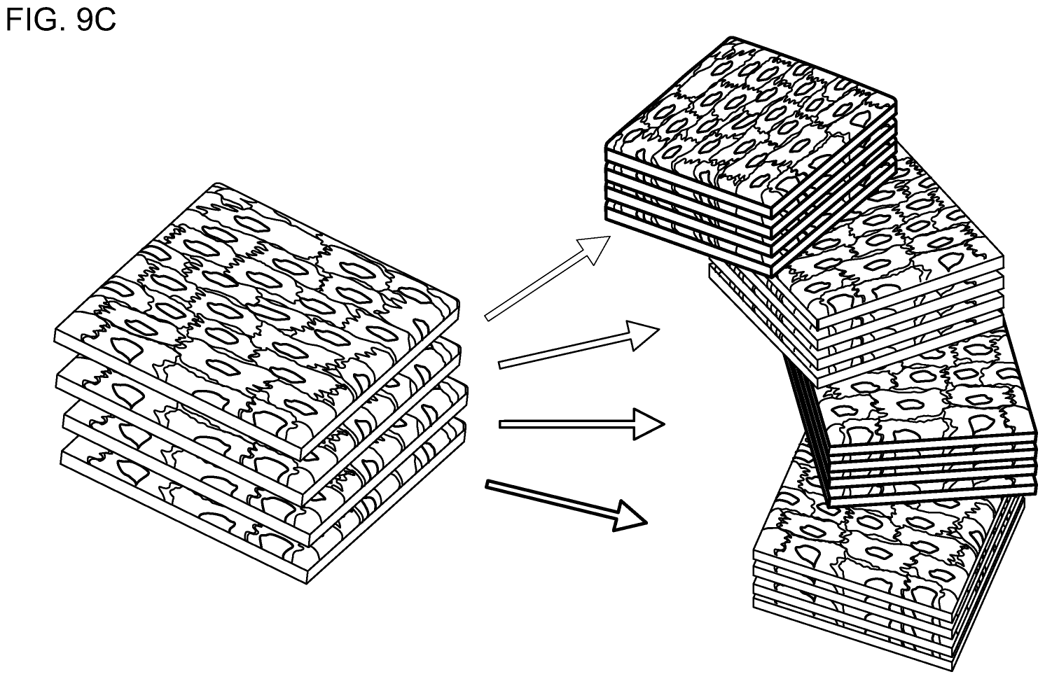

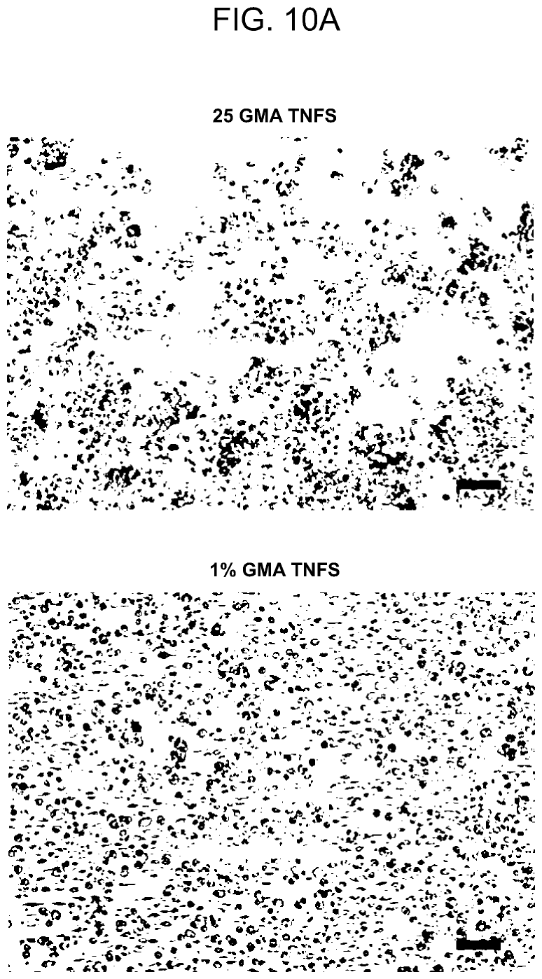

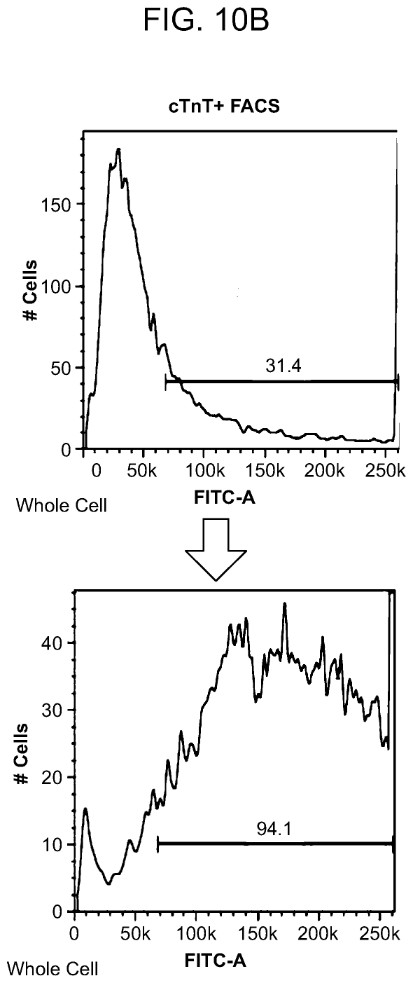

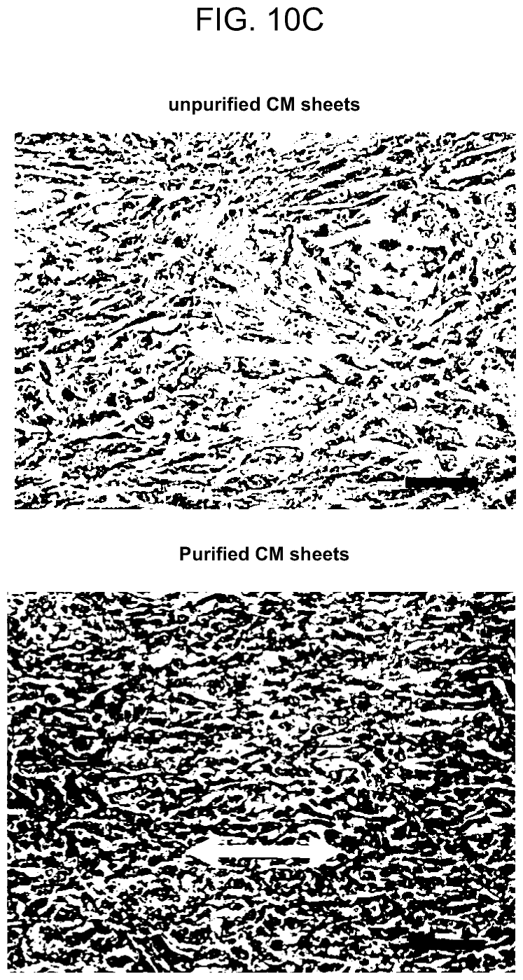

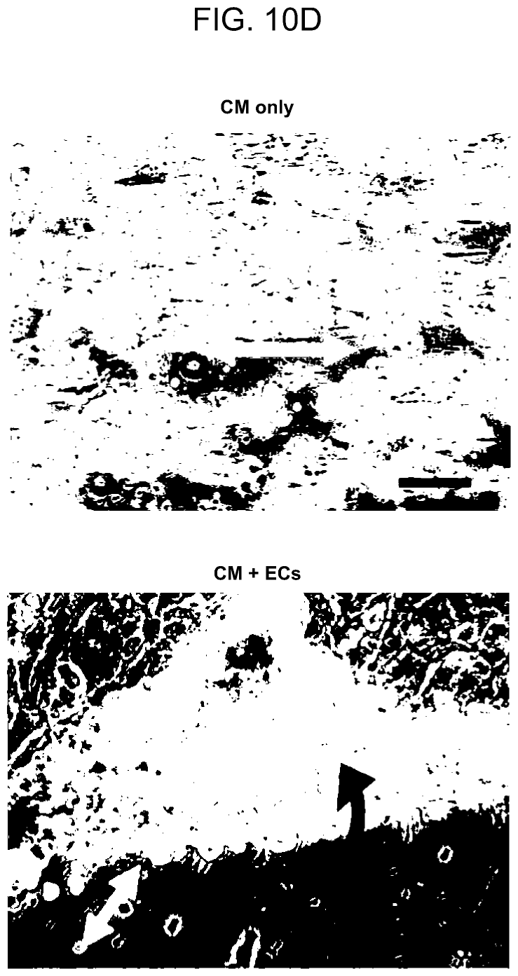

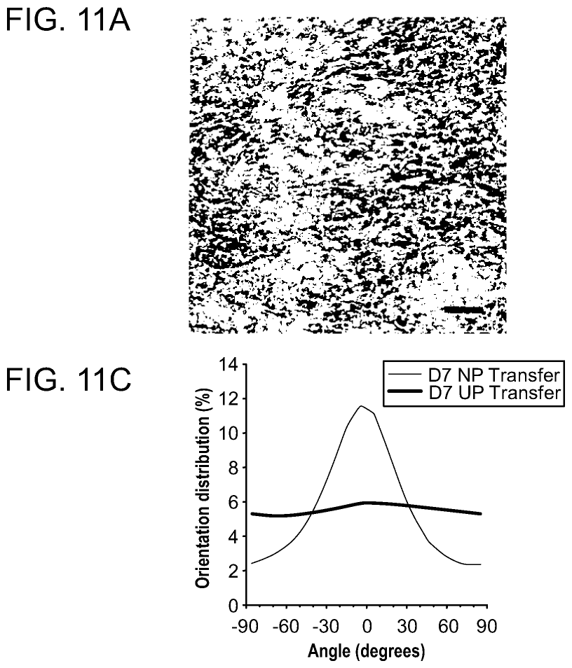

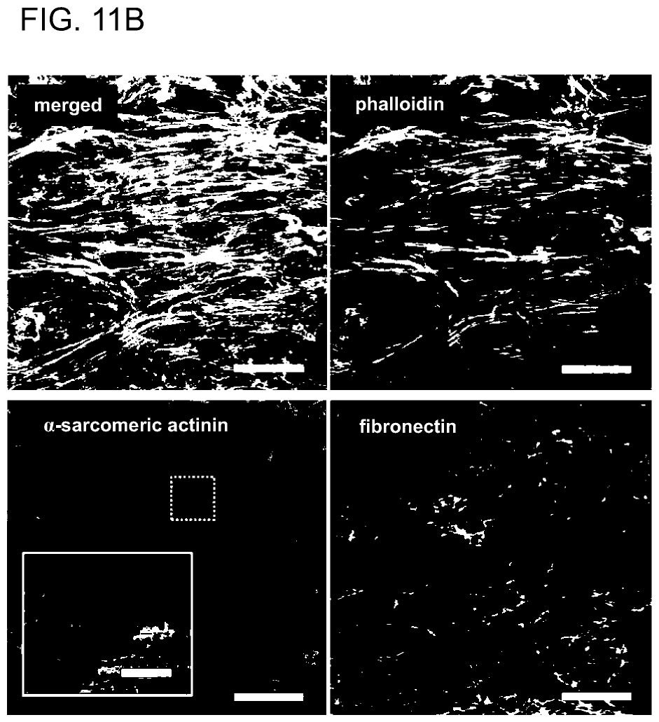

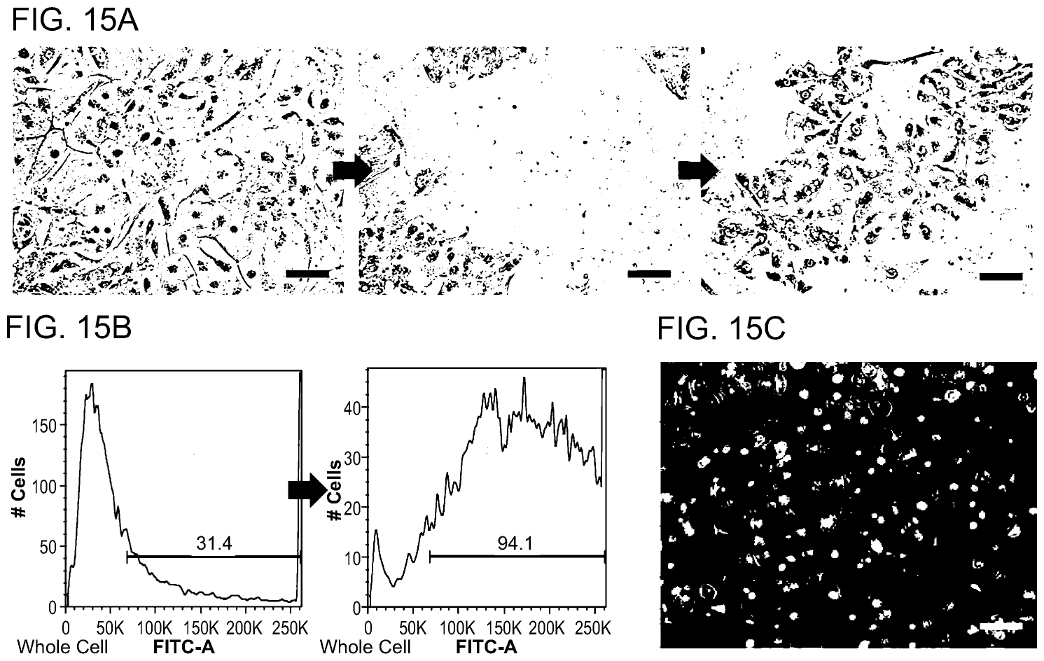

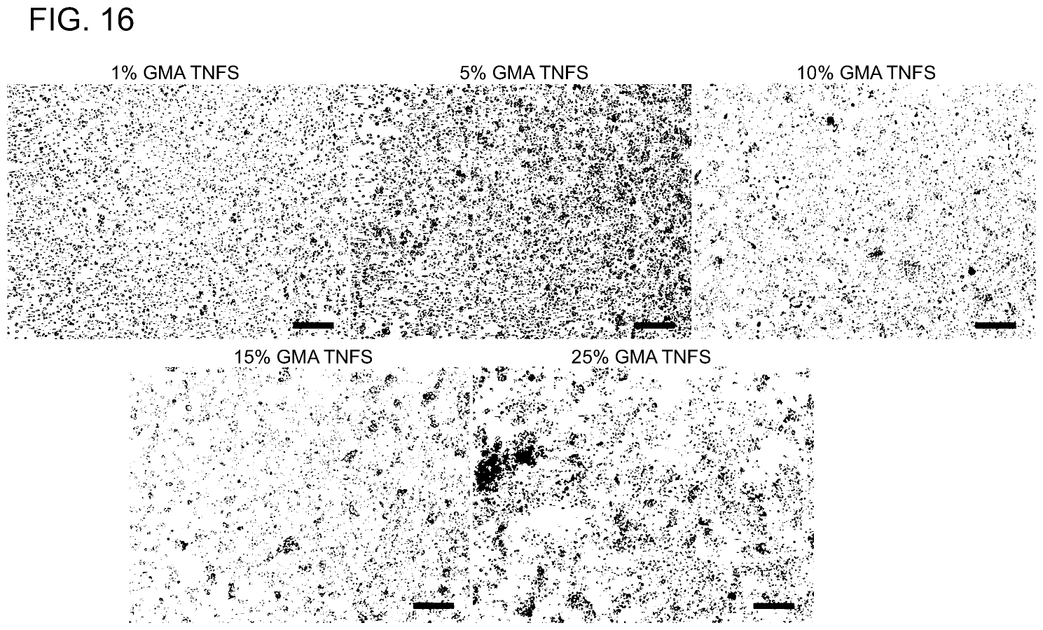

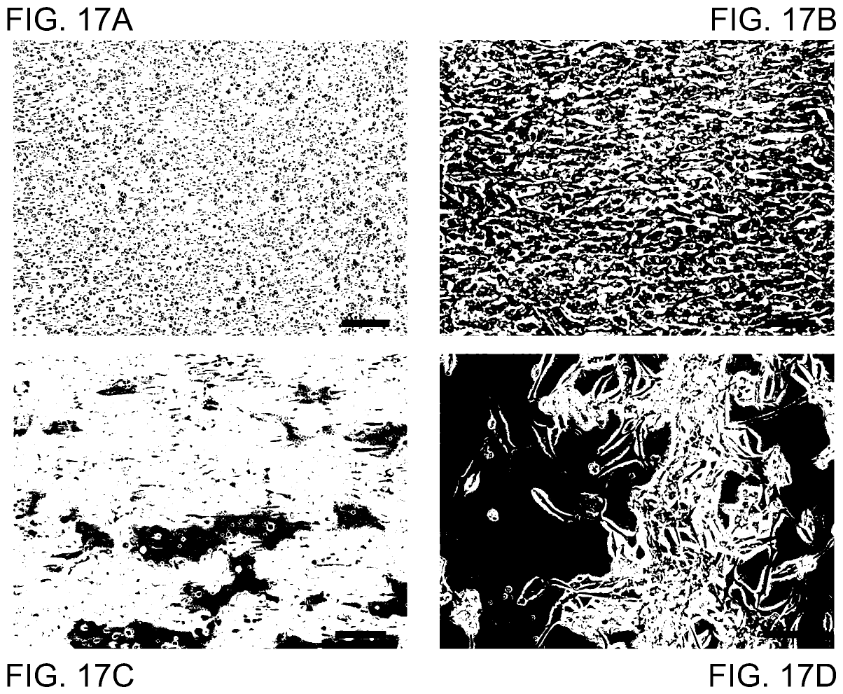

In order to ensure consistent formation of cardiac cell sheets, metabolic selection was incorporated to purify all cardiomyocyte differentiation runs. Using metabolic selection, non-cardiomyocytes detached within the first few days of purification, leaving highly pure populations of beating cardiomyocytes at the end of selection (FIG. 15A-15C), which were used for subsequent experiments. Next, to determine the appropriate surface chemistry parameters to allow for nanopatterned cardiac cell sheet engineering, altering the density of the grafted PNIPAM was investigated. The density of the grafted PNIPAM chains can affect the attachment and detachment of cell sheets, with too-densely grafted PNIPAM preventing formation of cell monolayers due to the hydrophobicity of the polymer. Pure (99% cTnT+) cardiomyocytes were seeded on 0.5%, 1%, 5%, 10%, 15% and 25% GMA v/v TNFS, with the GMA concentration affecting PNIPAM grafting density. Cardiomyocytes were able to form aligned cardiac monolayers on 0.5%, 1% and 5% GMA TNFS, but did not form confluent monolayers on 10%, 15% or 25% GMA TNFS (FIG. 16). Cardiomyocytes seeded on 0.5% and 1% GMA TNFS demonstrated syncytial beating monolayers and especially well-aligned cytoskeletons and defined sarcomeric striations after 7 days of culture on the TNFS (FIGS. 17A-17D). To test thermoresponsive detachment of cell sheets, TNFS were subsequently incubated with room-temperature DPBS to promote cell sheet detachment, however none of the tested conditions allowed for the detachment of intact cell sheets (FIGS. 10A-10C), with cells clumping together instead of detaching as intact sheets.

### Example 6: Endocardial-Like Endothelial Cell Incorporation is Necessary for the Thermoresponsive Detachment of Nanopatterned Cardiac Cell Sheets from the TNFS

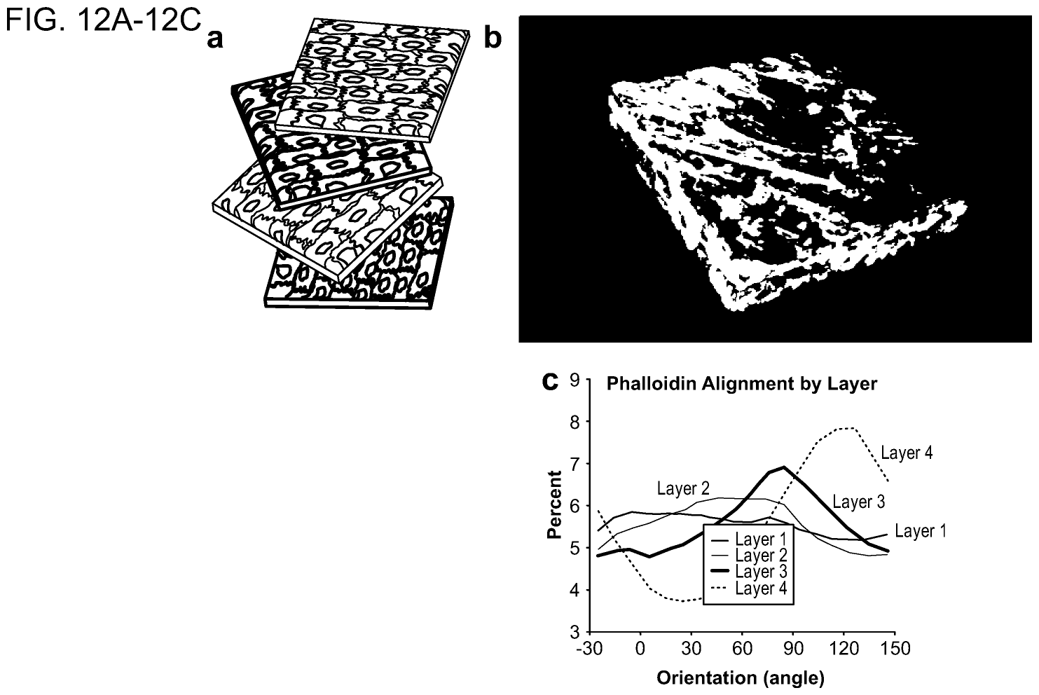

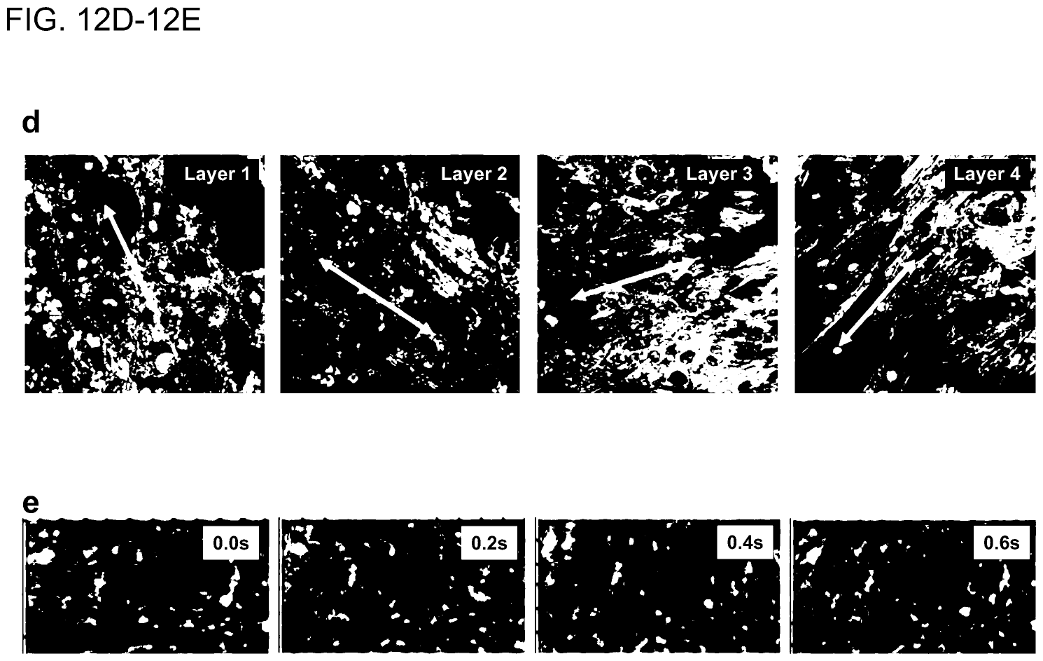

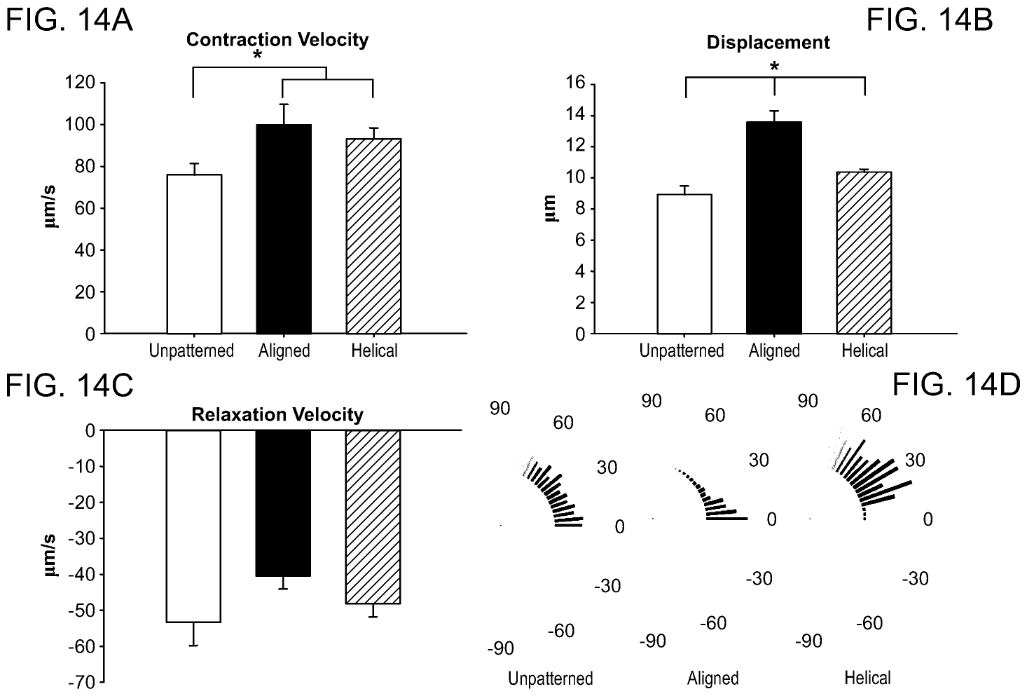

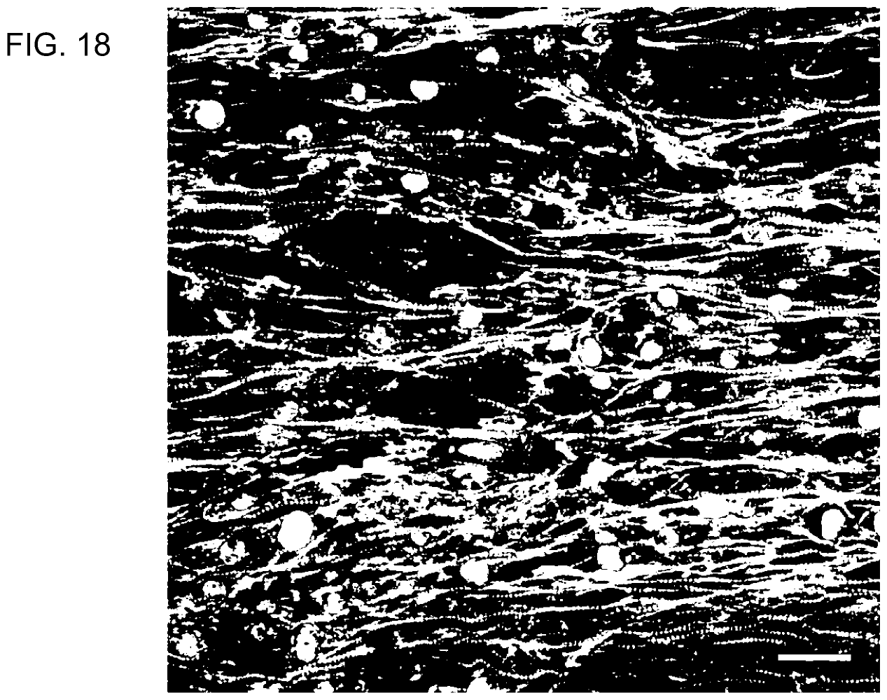

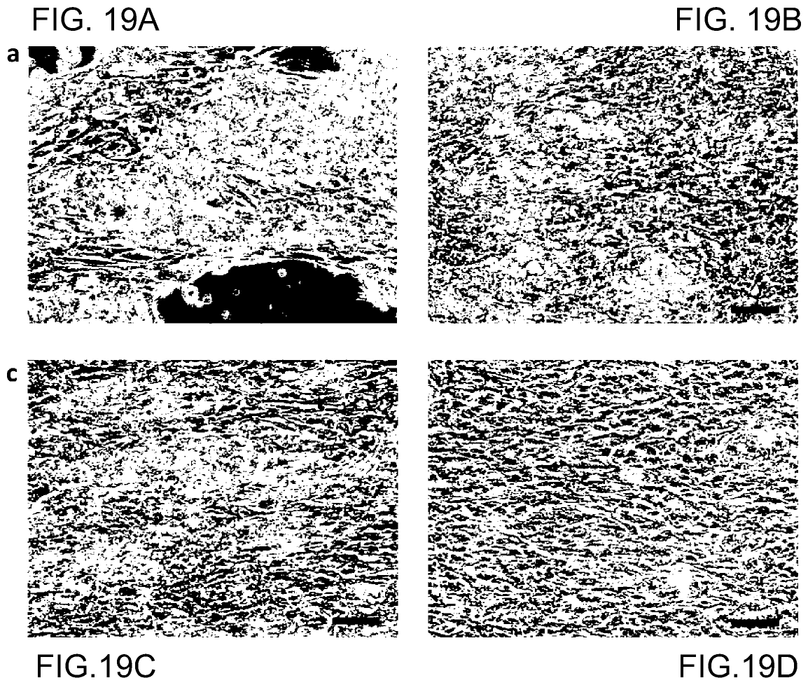

Previous studies have shown that extracellular matrix must be deposited by the cells to allow for an intact cell sheet to detach from a thermoresponsive surface, and after a change in culture temperature below the PNIPAM lower critical solution temperature (32° C.), the deposited ECM and cell monolayer detaches spontaneously while maintaining cell-ECM and cell-cell connections^{[41]}. To allow for deposition of ECM, four different stromal cell cocultures used in other cardiac tissue engineering studies were investigated towards the formation of detachable, nanopatterned cardiac cell sheets: primary human dermal fibroblasts (hDFs), stromal cell lines hs27a and hs5, and hiPSC-derived endocardial-like endothelial cells (ECs) in varying ratios. Stromal cell line hs5 did not allow for the formation of cardiac cell sheets (FIG. 16) on the TNFS, while hDFs, hs27as and ECs formed confluent, aligned cardiac monolayers (FIG. 17A-17D). The remaining three stromal cell conditions were incubated with room-temperature DPBS for 60 minutes to test for detachment capabilities. hDF cocultures were unable to be detached from the TNFS under any coculture ratio and additionally formed heterogenous tissues with nodes of aligned cardiomyocytes beating asynchronously (FIG. 18). Stromal cell line hs27a cocultures and EC cocultures detached spontaneously from the TNFS during DPBS incubation and were subsequently transferred using the gel-casting method, however hs27a cocultured cardiac cell sheets lost alignment after transfer (FIG. 19B). Only EC cocultured cardiac cell sheets were able to be transferred with maintained alignment (FIG. 19C). From these experiments, 0.5% and 1% GMA TNFS seeded with 1:5 EC:CM ratio allowed for formation of confluent, syncytial, anisotropic cardiac monolayers which could detach spontaneously from the TNFS surface with a reduction in culture temperature (FIG. 12A-12F, FIG. 18 and FIGS. 19A-19D). Additionally, the detaching nanopatterned cardiac cell sheet maintained cell-cell connections during detachment, as evidenced by synchronously beating detached cardiac cell sheets. As the cardiac sheets were cultured for up to 14 days on the TNFS and would continue to beat throughout culture, premature detachment of cardiac sheets was occasionally noted on 1% GMA TNFS. As a result, subsequent transfer and stacking experiments utilized 0.5% GMA TNFS and controls.

### Example 7: Transferred Nanopatterned Cardiac Cell Sheets Maintain Alignment Long Term and can be Stacked to Form Multilayered, Aligned Cardiac Issues with Discrete Cardiac Layer

To determine feasibility of engineering aligned cardiac tissues for further structure-function studies, transfer of single aligned cardiac sheets were investigated and then subsequently multilayered, stacked aligned cardiac tissues. Using the gel-casting method, nanopatterned cardiac cell sheets were transferred to a matrigel-coated coverslip. Transferred sheets maintained alignment and beating immediately after transfer (FIG. 13A) as well as long term, 7 days post-transfer (FIG. 13B). Analysis of cytoskeletal alignment using automated image analysis demonstrated maintained structural alignment relative to unpatterned controls (FIG. 13C). Morphological analysis of cardiomyocytes within the transferred nanopatterned sheet demonstrate well-ordered sarcomeres, demonstrating a more mature structural phenotype.

To engineer multilayered cardiac tissues, cardiac cell sheets were stacked using the gel casting method to generate 4-layered thick cardiac tissues either uniaxially aligned (aligned), helically aligned (helical), or unpatterned cardiac sheets as a control. Tissues were transferred to matrigel-coated coverslips and also maintained structure 7 days post-transfer. Interestingly, during stacking, individual sheets would contract simultaneously but were connected loosely enough such that individual sheets were discernible during contractions. After 24 hours of culture after transfer, however, the sheets contracted and relaxed simultaneously, indicating some degree of tissue compaction or tighter cardiac sheet connections after culture. To visualize individual layers of the engineered 4-layered cardiac tissues, green and red dyed cell sheets were stacked in alternating layers (FIG. 12A). Depending on culture time, 4-layer cardiac tissues either would have heterogeneous mixing of the individual cardiac sheets (7 day culture before transfer, FIG. 12B) or could maintain individual sheets integrity (14 day culture before transfer, FIG. 12B).

### Example 8: Engineered Multilayered Cardiac Tissues Retain Individual Layer Alignment Even When Stacked in Complex, 3D Tissues, Which Subsequently Affects Tissue Function

To assess intra-tissue structure, 4-layer cardiac tissues were immunofluorescently stained and imaged for structural, cardiac, and extracellular matrix proteins. Analysis of cytoskeletal alignment demonstrated maintained structural alignment in individual layers, however the degree of alignment would decrease from bottom to top (FIG. 12D, FIG. 13C). Cardiomyocytes also demonstrated well-ordered sarcomeres similar to the single nanopatterned sheets throughout the tissue, as well as presence of deposited extracellular matrix proteins (FIG. 12F). Using z-stacked confocal microscope images of the tissues, individual nanopatterned sheets were roughly 8-10 μm thick, with a total laminae thickness of ˜40 μm (FIG. 12C). No presence of vasculature was found during imaging of the cardiac tissues.

CCQ-based video analysis of tissue contractions indicated generally unidirectional contractile motion of the tissues for aligned tissues, with a swirling pattern for helical tissues (FIG. 12F and FIG. 13E). To assess if alignment of cardiac tissues could affect function, and if multilayered tissues functioned differently than single cardiac sheets, video recordings were analyzed of the contracting cell sheets and tissues during paced field stimulation. Transferred, aligned single cardiac sheets demonstrated improved contraction magnitude, contraction velocity and relaxation velocity over controls with a less disperse angle of contraction (FIG. 6A-6D). All three endpoints further increased with the multilayered aligned and helical cardiac tissues over single sheet controls and multilayered unpatterned controls, with aligned tissues demonstrating the greatest improvement in contractile function.

### Discussion

In the present study, a robust, “bottom-up” approach is shown for engineering cell-dense cardiac tissues that allows for precise control of 3D tissue structure. By utilizing biomimetic nanotopographical cues, entire cardiac monolayers were aligned. With the incorporation of a thermoresponsive release layer, as well as modifying tissue parameters such as the inclusion of a stromal cell population to allow for ECM deposition, the anisotropic cardiac sheets can be detached and transferred without loss of structure. Even more promisingly, the anisotropic sheets can be stacked to form multilayered cardiac tissues with a variety of tissue structures. The stacked nanopatterned cardiac sheets are able to beat in sync with one another while maintaining individual sheet anisotropy, allowing for the fabrication of both aligned and helical 3D cardiac tissues. Further, the different 3D cardiac tissue structures also demonstrated different contractile properties, highlighting the importance of overall cardiac tissue structure on tissue function even at the scale of individual cardiac sheets.

Although the heart contains an abundant amount of structurally organized extracellular matrix proteins, the myocardium is a cell-dense tissue. Cardiomyocytes must be in direct contact with one another to transmit an action potential and transmit force during a contraction^{[42,43]}. As a result, the use of scaffolds to engineer cardiac tissue often limits the engineered tissue utility due to inflammatory response of implanted materials as well as difficulties with host tissue integration^{[23,44]}. The advent of scaffold-free cardiac tissue engineering has yielded promising results, specifically showing improvements in cardiac function after transplantation, but all scaffold-free, engineered cardiac tissues thus far have lacked structural organization^{[45, 46]}. The engineered, structured, 3D cardiac tissues demonstrated improved contractile properties over unstructured controls. For aligned 3D cardiac tissues, the alignment of contractile direction, and subsequently, contractile force, would have an obvious additive effect. However, interestingly, even helically structured 3D cardiac tissues demonstrated improved contractile properties over controls, even though the top and bottom layer were oriented nearly perpendicularly. This could potentially be due to the fact that individual cardiac sheets in the helical tissue were still structured and therefore had an anisotropic, uniaxial direction of contraction, whereas control tissue contraction vectors were isotropic. This could lead to a greater overall contraction magnitude and velocity when summed throughout the 3D cardiac tissue, albeit blunted as the individual cardiac sheets were not aligned in a single direction.

Another additional interesting finding is the decrease in alignment of individual layers from the bottom to the top. This is likely due to decreasing mechanical stress, with the bottom layer affixed to a rigid glass substrate, while the top layer is attached to another cell sheet. Mechanical stress modeling could be a promising tool in analyzing the specific physical forces experienced by cell sheets in 3D tissues to confirm this finding. Additionally, a rigid top backing, such as the intra-laminae ECM in the heart, could be used to provide passive tension to maintain top layer orientation. However, despite the decrease in the magnitude of alignment on the top layer, overall 3D tissue structure still impacted contractile function, highlighting structure-function relationship of cardiac tissue even within 40 μm tissue constructs. As the human myocardium is approximately 200 times thicker than these laminae constructs^{[47]}, these constructs could be promising unit models of larger tissue, and possibly whole organ function.

Additionally, the ability to engineer more complex 3D cardiac tissue structures, like a helical architecture, also allows for more robust exploration of the role of cardiac structure into additional fields, such as developmental biology. For instance, although the adult cardiac structure is well-documented, the underlying processes regulating the development of this structure are still under active research. Fibronectin and other ECM components assist in migration of cardiac precursors early in heart development^{[48]}, followed by elongation of individual cardiomyocytes which form lateral cell-matrix connections to aligned ECM fibers^{[49]} and then self-organization of aligned fiber tracts into a helical structure late in fetal development and progressing through postnatal development^{[50]}. These in vivo studies have suggested that both the ECM and the 3D cardiac microenvironment may contribute to the maturation of cardiomyocytes as well as the structural organization of the myocardium. The platform could subsequently allow for the analysis of cardiac microenvironmental effects, including structure, on the development of embryonic stem cell-derived cardiomyocytes. Subsequently, as the platform was used with a variety of other cell types, including cells derived from similar developmental lineages as cardiomyocytes, complex, multilayered cardiac tissues are engineered, comprised of endocardium, myocardium, and epicardium, for the analysis of cardiomyocyte and supporting cell interactions during development. The engineering of a variety of cardiac tissue structure thus could provide interesting insights into stem cell biology and development in addition to advancing tissue engineering for clinical purposes.

### Methods

### Thermoresponsive Nanofabricated Substratum (TNFS) Fabrication

The thermoresponsive nanofabricated substrate was fabricated as described^{[35]}. Briefly, a polyurethane acrylate (PUA, Norland Optical Adhesive) and epoxy-containing glycidyl methacrylate (GMA, Sigma-Aldrich) solution was mixed together and utilized in capillary force lithography to fabricate nanotopographical substrata as previously published^{[19]}. Once polymerized, the substrate was incubated with an amine-terminated PNIPAM solution (Mn: 2500, Sigma-Aldrich) in DI H₂O and allowed to react for 24 h on a rocker at room temperature. The GMA percentage was varied (0.5%, 1%, 5%, 10%, 15%, 25% v/v) to change the PNIPAM grafting density.

Derivation and Differentiation of Human Induced Pluripotent Stem Cells (hiPSCs) into Cardiomyocytes and Endothelial Cells.

Informed consent was obtained from all patients as directed by the Institutional Review Board (IRB) policies. Urine cells were isolated and expanded from a single healthy male participant as previously described^{[36]}. A polycistronic lentiviral vector encoding human Oct3/4, Sox2, Klf4, and c-Myc4 was used to reprogram the urine cells into iPSCs. The derivative hiPSC line was karyotyped and shown to be a normal 46, XY karyotype and was subsequently used for differentiation. A modified monolayer-based directed differentiation method was used as previously published for cardiomyocytes^{[7]}. Briefly, the day prior to induction, undifferentiated hiPSCs were treated with mTeSR 1 media (Stem Cell Technologies) supplemented with CHIR-99021 (Selleck). On the day of induction, undifferentiated hiPSCs were treated with RPMI-1640 media supplemented with B-27 without insulin and activin A (R&D Systems) and matrigel (BD Biosciences). 18 hours post-induction, the media was exchanged for media supplemented with BMP4 (R&D Systems) and CHIR-99021. Cells were then fed on day 3 with cytokine-free RPMI-1640 media supplemented with B-27 without insulin (ThermoFisher) and XAV-939 (Tocris Bioscience), on day 5 with cytokine-free RPMI-1640 media supplemented with B-27 without insulin, and then finally on day 7 and every other day thereafter using RPMI-1640 media supplemented with B-27 with insulin. Beating cells were first seen at ˜7 days post-induction, cultured for 7 more days, and then were subsequently split and seeded at a lower density (100 k cells/cm²) into a new culture dish for cardiomyocyte purification using metabolic selection as previously published^{[37]}. Cardiomyocytes used for subsequent experiments were 90% cTnT+ or higher and used between 28 to 35 days post-induction. For differentiation into endothelial cells, a similar monolayer-based directed differentiation method was used as previously published^{[38]}. Briefly, after cytokine treatment, media was switched to StemPro media supplemented with ascorbic acid, BMP4, bFGF and VEGF for 3 days. Cells were split at day 5 post-induction and then fed with endothelial-specific media, EGM supplemented with CHIR-99021, bFGF and VEGF, to induce an endothelial phenotype. ECs were analyzed at 5 days post-induction to determine CD31+ purity via FACS and used at 14 days-post induction and were 85% CD31+ or higher and were maintained in endothelial-specific media.

### Culture of Cardiac and Stromal Cells on the TNFS for Cardiac Cell Sheet Engineering

Human bone marrow-derived stromal cells hs27a and hs5 (Lonza) were thawed and maintained according to manufacturer's instructions. Human dermal fibroblasts (hDFs) were acquired via a skin punch biopsy from the forearm of a healthy 52-year old male. Endocardial-like endothelial cells (ECs) were differentiated and maintained as described above. For cardiac cell sheet seeding, hiPSC-derived cardiomyocytes and stromal cells were split from their culture plates using 0.25% trypsin/EDTA (Lonza) and resuspended and mixed at stromal cell concentrations of 10%, 20% and 30% and seeded onto fibronectin-coated (5 ug/cm2) TNFS at a seeding density of 175,000 cells/cm². Cells were cultured using a 1:1 mix of RPMI-1640 media with B-27 supplementation (Lonza) and EGM (Lonza) and cultured for 7 days after seeding. For fluorescent labeling of specific cell sheets, cells were suspended for 30 minutes in serum-free media supplemented with 2 uM CellTracker Green or Red (ThermoFisher) prior to seeding.

### Transfer and Stacking of Nanopatterned Cardiac Cell Sheets Using the Gel-Casting Method.

Cell sheets contract upon detachment from the TNFS surface and require a method to transfer sheets without loss of cell morphology. The gel-casting method was used as described^{[35]} to transfer and stack nanopatterned cardiac cell sheets. Briefly, cell-seeded TNFS were incubated with room-temperature DPBS for 30 minutes to initiate cardiac cell sheet detachment. Prior to full sheet detachment, the DPBS was aspirated and melted 37° C. 7.5% w/v gelatin (Sigma-Aldrich) in media was added to the TNFS and then casted at 4° C. for 15 minutes to firm the gelatin and prevent full sheet detachment and subsequent compaction. The TNFS was then incubated at 28° C. for 1 hour to allow for full cardiac cell sheet detachment. The gel-casted nanopatterned cardiac cell sheet could then be transferred to a new surface, such as a plasma-treated (100 W, 5 minutes), matrigel-coated glass coverslip, or onto another cell-seeded TNFS and incubated for 2 hours at 28° C. to stack multilayered cardiac tissues. The stacking process was repeated up to 4 times to generate 4-layer thick nanopatterned cardiac sheets, which were then transferred to plasma-treated, matrigel-coated glass coverslips for subsequent culture. 4-layered cardiac tissues were either structured to have uniaxial alignment (aligned), 20° separation between individual cardiac sheets (helical), or were unpatterned controls.

### Immunostaining, Imaging and Image Analysis of Cardiac Cell Sheets

Cells were washed with PBS (Sigma) and fixed in 4% paraformaldehyde (Sigma) for 15 min at room temperature (22° C.). Fixed cells were then washed with PBS and permeabilized and blocked with a solution of 5% bovine serum albumin (BSA, Sigma) and 0.25% Triton X-100 (Sigma) in PBS for 1 h at room temperature, then washed with PBS. Multilayered sheets were permeabilized and blocked for up to 4 hours. Cells and cell sheets were then incubated with primary antibodies to α-sarcomeric actinin (1:200, Abcam), fibronectin (1:1000, Abcam), or CD31 (1:20, Abcam) in 1% BSA in PBS overnight at 4C. For secondary antibody labeling, AlexaFluor488-conjugated phalloidin (1:200, Invitrogen) and the appropriate AlexFluor-conjugated secondary antibodies (Invitrogen) in a 1% BSA in PBS solution were added to the cells and cells sheets for 1 hour at 37° C. All samples were then stained with a Hoechst stain (Sigma) at a dilution of 1:1000, washed with PBS once, then treated with Vectashield (Vector Laboratories), mounted on coverslips, and imaged using a confocal microscope. Imaging studies were supported by the Mike and Lynn Garvey Cell Imaging Lab at the Institute for Stem Cell and Regenerative Medicine at the University of Washington. Images were collected on a Nikon A1 Confocal System attached to a Nikon Ti-E inverted microscope platform. To quantitatively assess cytoskeletal alignment, confocal microscopy images of phalloidin or CellTracker-stained cells were taken at three representative fields at 60× magnification and analyzed using a modified, MATLAB script utilizing pixel gradient analysis as previously published^{[35, 39]}.

### Correlation-Based Contraction Quantification (CCQ) Analysis of Cardiac Cell Sheet Contractile Function

In order to assess contractile function, videos of paced, contracting cell sheets were used. To acquire the videos, media was replaced with warmed Tyrode's solution and the cardiac sheets were field stimulated using the MyoPacer Field Stimulator (Ionoptix) at 1 HZ, 10V, square waves with a 5 ms duration. Videos of at least 4-5 contractions and 3-5 fields of view per sample were then analyzed. For the analysis, the CCQ method was used as previously published to quantify contractile function of cardiac cell sheets^{[40]}. Briefly, a reference video frame is divided into a grid of windows of a set size. Each window is run through a correlation scheme with a second frame, providing the new location for that window in the second frame. This displacement is converted into a vector map, which provides contraction angles and, when spatially averaged, contraction magnitudes and velocities. The co-relation equation used provides a Gaussian correlation peak with a probabilistic nature that provides sub-pixel accuracy. The videos used to perform this analysis were taken with a 60 FPS.

### Statistical Analysis

Statistical significance between unpatterned control cardiac cell sheets and nanopatterned cardiac cell sheets was determined using Two-Way ANOVA with Tukey's pairwise post-hoc analysis using SigmaPlot software unless otherwise stated. For the contraction angle analysis, a Chi square test run at 5% significance was utilized to quantify uniformity in alignment distributions. This test was calculated using MATLAB. For all statistical analyzes, a p-value less than 0.05 was considered significant. Error bars are standard error mean (SEM).

### Example 9: Fabrication of Bioinspired Tissue-Engineered Cardiac 3D Ventricular Models

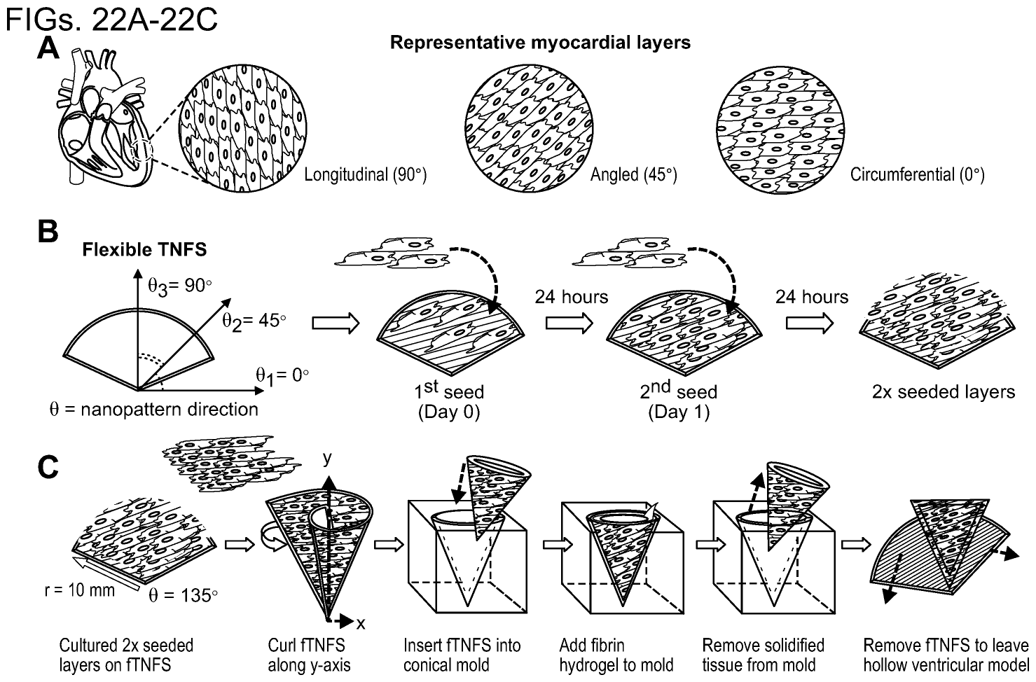

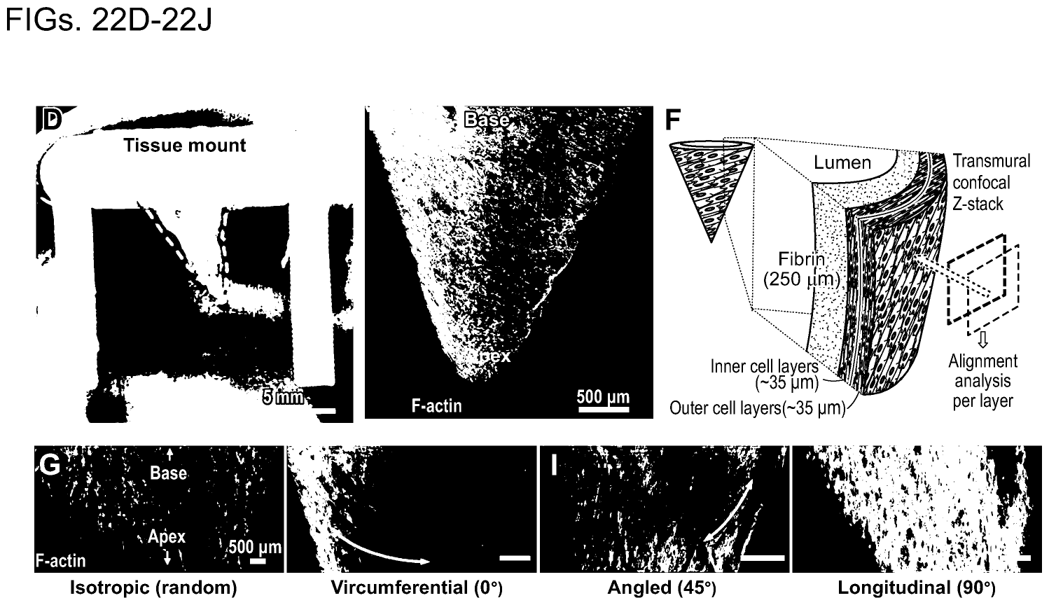

To investigate the 3D macroscopic structure-function relationships within the human myocardium, flexible thermoresponsive nanofabricated substrates (fTNFS) were used to engineering scaled models of the human left ventricle (FIGS. 22A-22J). The flexible TNFS were cut into fan shapes such that the nanoridges and grooves were oriented in at 90°, 45°, or 0° angle relative to the scaffold's long axis (FIGS. 22A and 22B). These fan-shaped sheets were subsequently rolled into conical structures to produce 3D ventricular models with longitudinally, angled, or circumferential cellular patterning, respectively. Unpatterned or topographically flat scaffolds were utilized to create models with random or isotropic cellular organization as a control. Specifically, each fTNFS was double seeded with induced pluripotent stem cell-derived (iPSC) cardiomyocytes and endothelial cells to form aligned cardiac sheets that exhibited coordinated spontaneous contraction patterns within 5 days of culture.

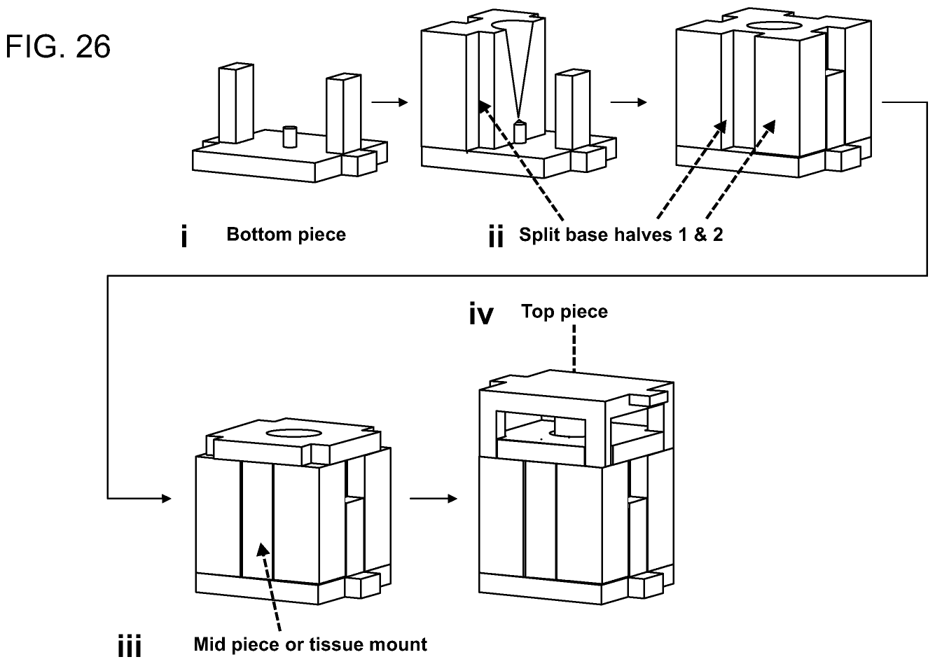

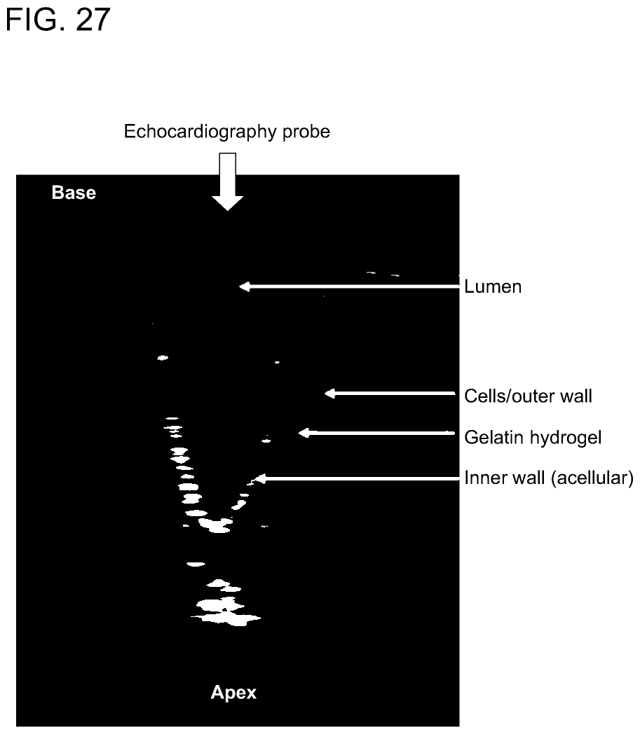

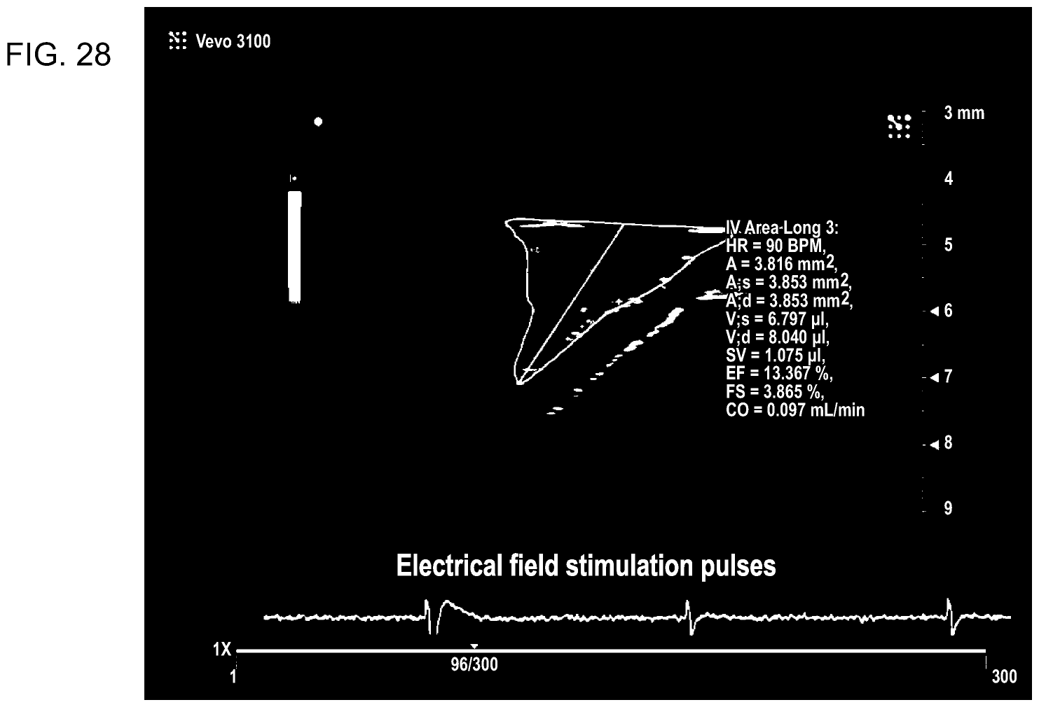

The organized cardiac sheets were casted with a fibrin hydrogel (20 mg/mL) and custom molds (FIG. 26) to fabricate hollow ventricular models that were on scale with the mouse heart16. The final dimensions were 7 mm in height, 5 mm in diameter at the base and tapered to a rounded point at the apex (FIG. 22C-22E). Within one hour after removal from the mold, the isotropic, circumferential, and longitudinal tissues exhibited coordinated spontaneous contractions in which the apex was pulled upward and inward towards the base of the tissue. Whereas the angled tissues exhibited an upwards twisting motion in the direction of the cellular patterning. This coordinated spontaneous contractions of each tissue suggested the cell layers were intact and formed a syncytium (FIG. 22E). The tissue wall thickness was approximately 320 μm, consisting of a 250 μm-thick fibrin wall encircled by 2 layers of cells (50-70 μm thick) (FIG. 22F).

### Example 10: Multi-Scale Control of Patterned Cell Organization within 3D Ventricular Models

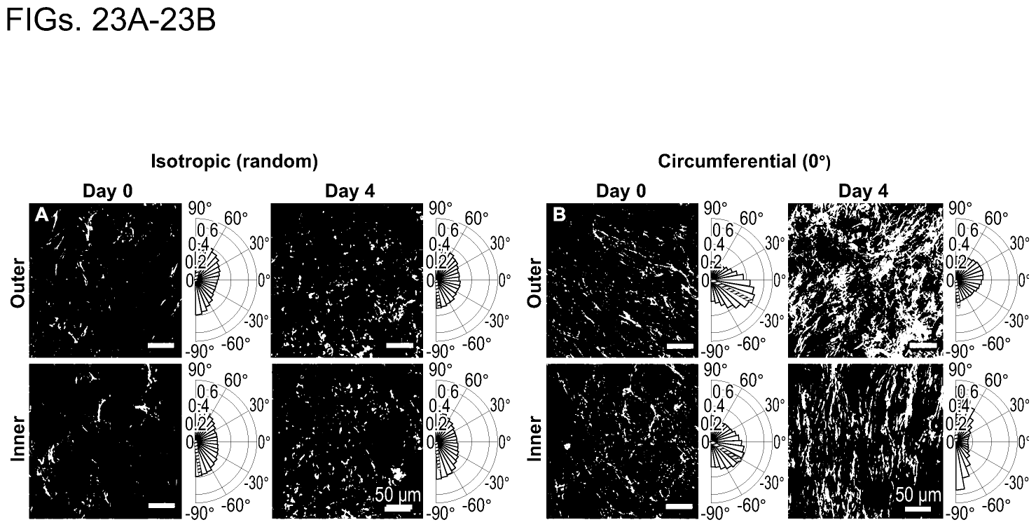

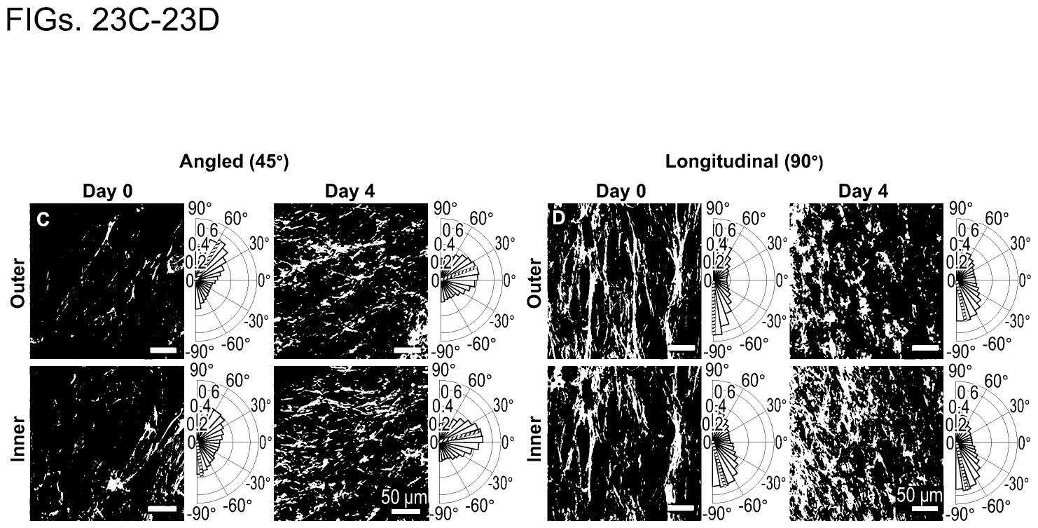

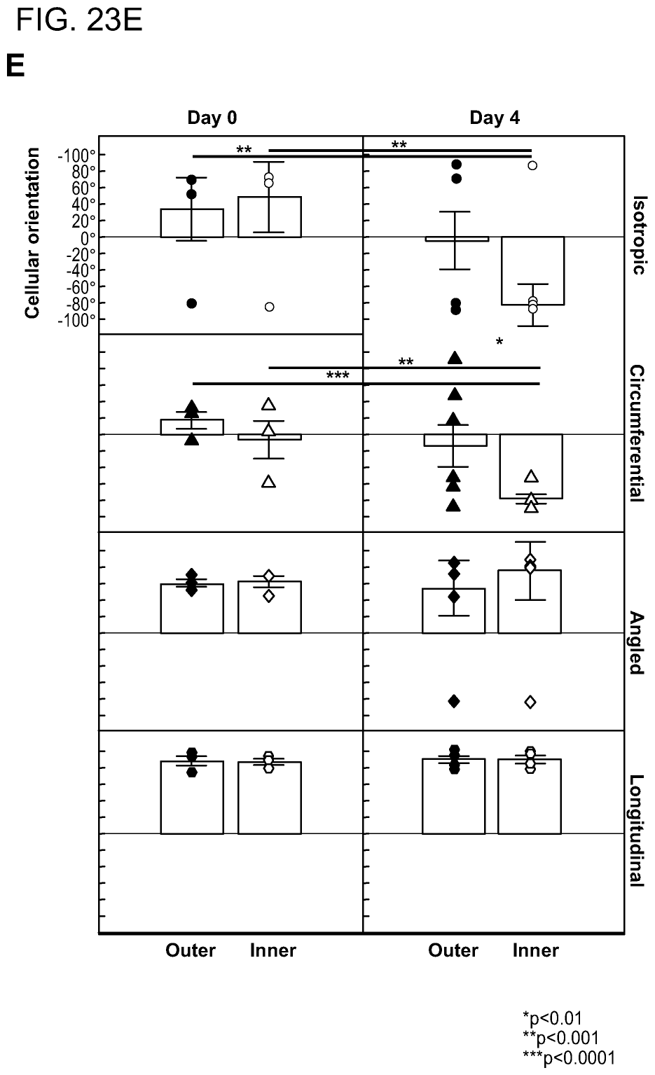

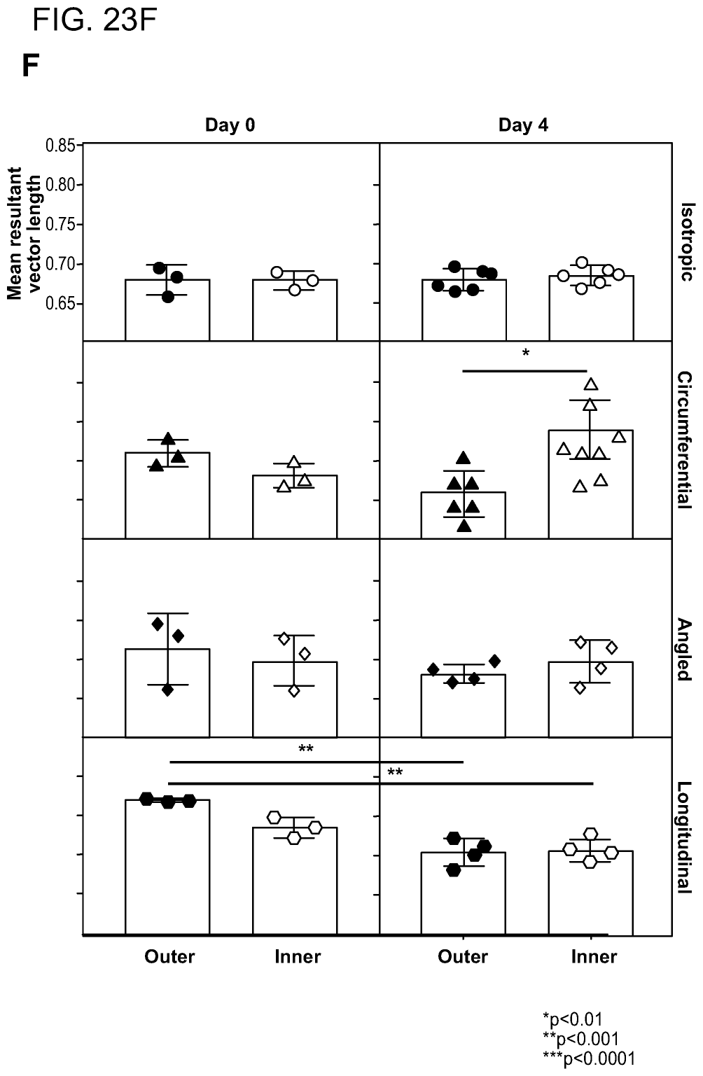

Immediately after the tissue casting process (day 0), ventricular models were fixed and stained to evaluate their macro- and microscopic cellular organization. High-magnification, confocal z-stacks were taken across the entire tissue area and transmurally through the cell layers. To determine if cellular organization was uniform, the outer and inner layers (˜35 μm each, 2 cell-layers thick) of the z-stacks were parsed, analyzed separately, and compared. On day 0, the average cellular orientation for all cell layers for the circumferential (intended angle=0°), angled (intended angle=45°), and longitudinal (intended angle=90°) groups were of 4.8°±7.4°, 60.9°±3.2°, and 87.1°±2.4° standard error of the mean (SEM), respectively (FIG. 23A-23F). The isotropic group demonstrated some direction bias towards 40.8°±15.6° SEM on average, but the distribution of the cellular orientations was very wide and lacked a prominent peak as compared to the other groups (FIG. 23E). Across all groups, there was little to no difference between the mean angle of the inner and outer layers on day 0 (FIG. 2E, Day 0 column). However, the global or mean angle of alignment did not provide information about how strongly coordinated the cellular alignment angles around the mean were. Therefore, the mean resultant vector lengths (RVL) for each layer's angle distribution was compared (FIG. 23F, Day 0 column). In other words, a RVL closer to 1 indicates a larger proportion of cells align in the mean direction whereas a RVL closer to 0 indicates a lack of coordinated alignment in the mean direction. When the RVLs of the outer and inner cell layers were compared within each group, no significant difference between the two was found. These data suggest that the intended pattern of global cellular alignment was maintained after the tissue fabrication process and that uniform organization was maintained across all cell layers.

### Example 11: Cellular Remodeling at Inner Most Cells Layers of Circumferentially Patterned Tissues

To determine if cellular patterning in the ventricular models were maintained over the culture period, issues were fixed after 4 days in culture and analyzed their cellular alignment (FIG. 23A-23F, day 4 columns). Tissues patterned with longitudinal or angled cellular alignment exhibited little cellular reorganization during the culture period for both the inner and outer cell layers (FIG. 23E). For the angled tissue group, the mean resultant vector lengths (RVLs) that describe the strength of coordinated alignment around the mean were also not significantly different over time (FIG. 23F). Additionally, the longitudinal tissues appeared to have lost some strength in alignment compared to day 0, although the principle direction of organization was nearly unchanged. Tissue patterned isotropically or with random cellular alignment exhibited almost no cellular remodeling during the culture period on the outer cell layers but there was some bias towards longitudinal cellular organization (Day 4, inner layer=−81.6°±25.9° SEM). However, the strength of cellular organization around the mean angle was still low (Day 4 RVL, outer/inner=0.68±0.005/0.68±0.005 SEM) and not significantly different than that of day 0 (Day 0 RVL, outer/inner=0.68±0.01/0.68±0.006 SEM).

Interestingly, the tissues with circumferential patterning exhibited a distinct degree of remodeling over the culture period. The inner cell layers reorganized to become strongly longitudinally aligned (Day 4, inner layer=−78.1°±5.3° SEM), which was nearly perpendicular to their original orientation on day 0 (Day 0, inner layer=−6.6°±22.8° SEM) (FIG. 23E). In contrast, the outer cell layers shifted slightly but held to their circumferential pattern (Day 4, outer layer=−14.1°±26.4° SEM). However, the strength of alignment of the outer cell layers became more diffuse as indicated by small RVL values (Day 4 RVL, outer layer=0.65±0.01) as compared to the significantly larger values of the inner layers (Day 4 RVL, inner layer=0.74±0.01) (FIGS. 23E and 23F).

### Example 12: Transmural Gradients of Shear Force and Strain in 3D Ventricular Models

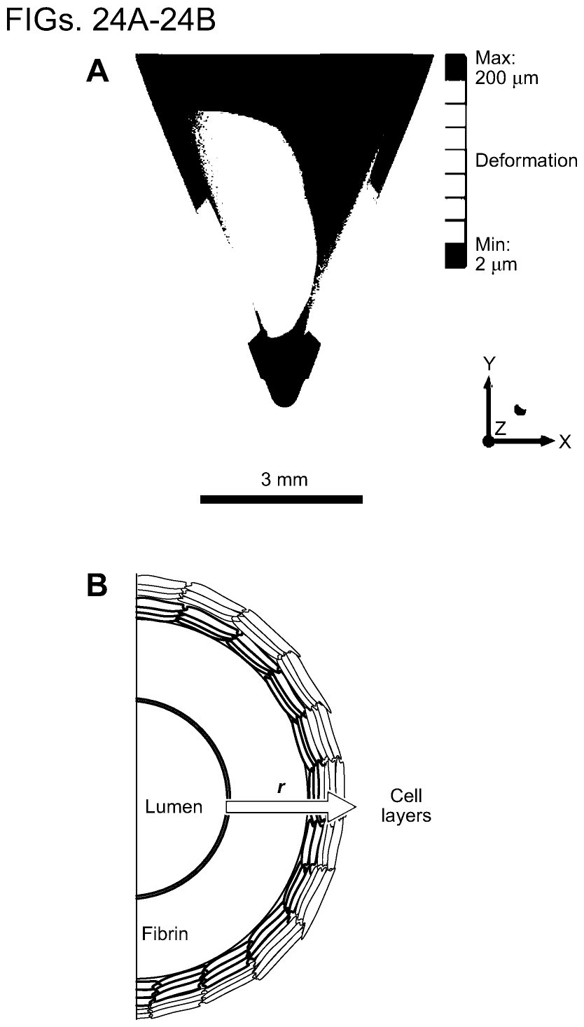

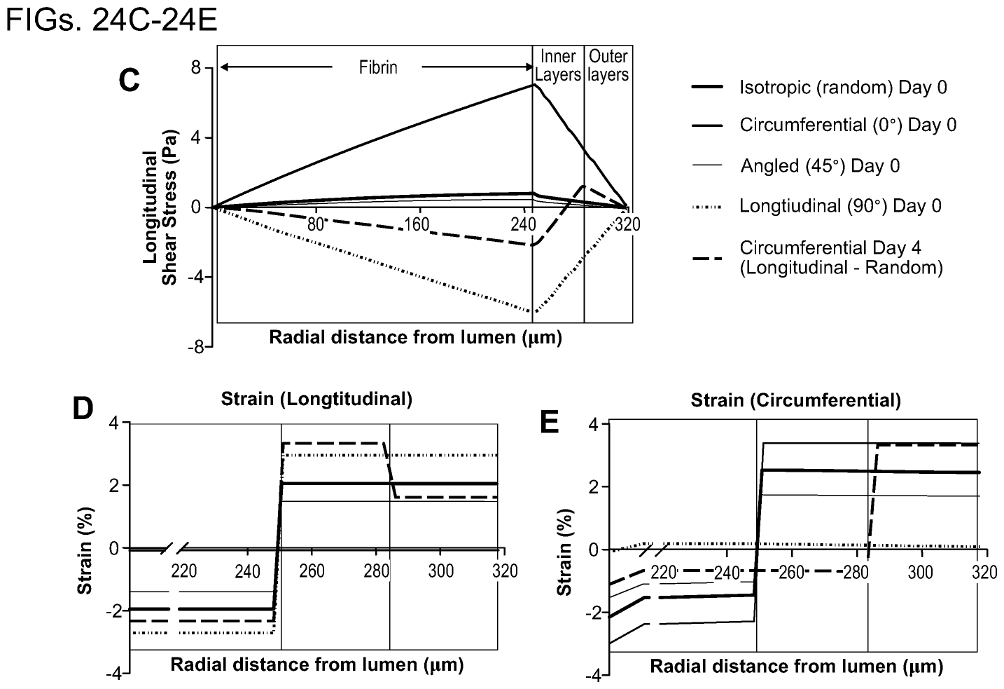

The striking remodeling effect that the observation in the circumferentially patterned tissues suggested that their cellular organization and therefore their contraction patterns may be providing mechanical cues that might promote remodeling. There might be differences in the transmural patterns of stresses and strains created by circumferentially patterned tissue contraction as compared to the other tissue architectures. Therefore, the system was computationally modeled to test and understand the mechanical forces at play between tissues with different architectures. To test this hypothesis, finite element model was built to look at different stresses and strains that might occur across the wall thickness in each of the conditional models at day 1 in culture. The model included the average tissue dimensions, stiffness of the fibrin hydrogel, wall thicknesses of the fibrin and cell layers, and the different cellular organizations (circumferential (0°), angled (45°), longitudinal (90°), and isotropic (random)) (FIGS. 24A and 24B). The model with experimental changes in tissue length from base to apex during contraction and relaxation observed in the longitudinally patterned tissues on day 1. This model was then used to predict the longitudinal shear stress (FIG. 24C), longitudinal strain (FIG. 24D), and circumferential strain (FIG. 3E) for each pattern group.

From these simulations, tissues with isotropic or angled patterns were found to have almost identical levels of stress and strain across the thickness of the tissue wall. It was interesting to learn that very little shear stress was developed across the cell layers in either of these tissue organizations as compared to the other two experimental conditions. Remarkably, longitudinal shear stresses were observed to be greatest, but opposite, at the interface between the fibrin hydrogel and the inner-most cell layers of the longitudinal and circumferential models (at 250 μm from the tissue lumen) (FIG. 3C). Similarly, the longitudinal and circumferential strains were also equal but opposite for these two groups (FIGS. 3D & E). These results combined with structural analysis on day 4 (FIGS. 23B, 23E and 23F) suggested that the initial circumferential cellular patterning and contractions created large shearing forces that were perpendicular to the cellular organization at day 0. Furthermore, these large perpendicular shear forces occur at the cell-fibrin interface and promoted cellular remodeling to align with the direction of the shearing, or along the long axis of the tissue.

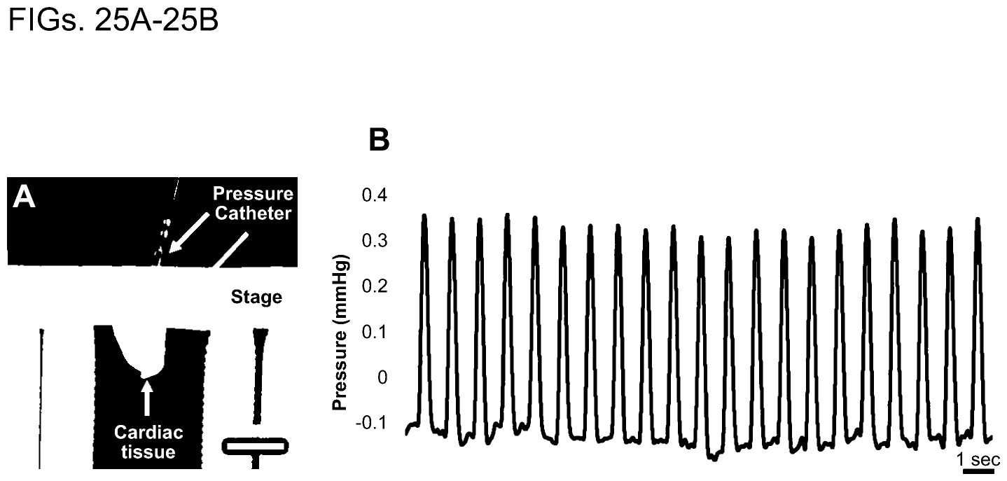

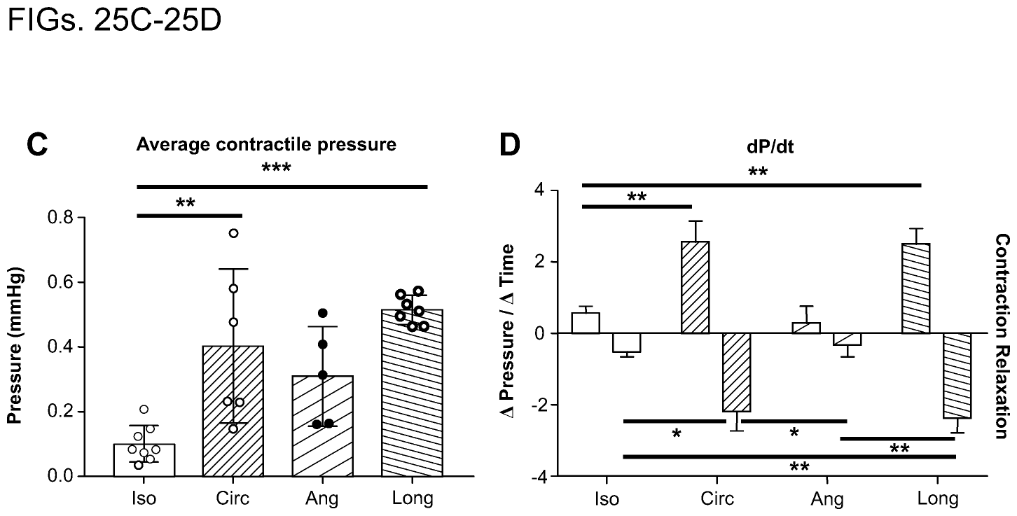

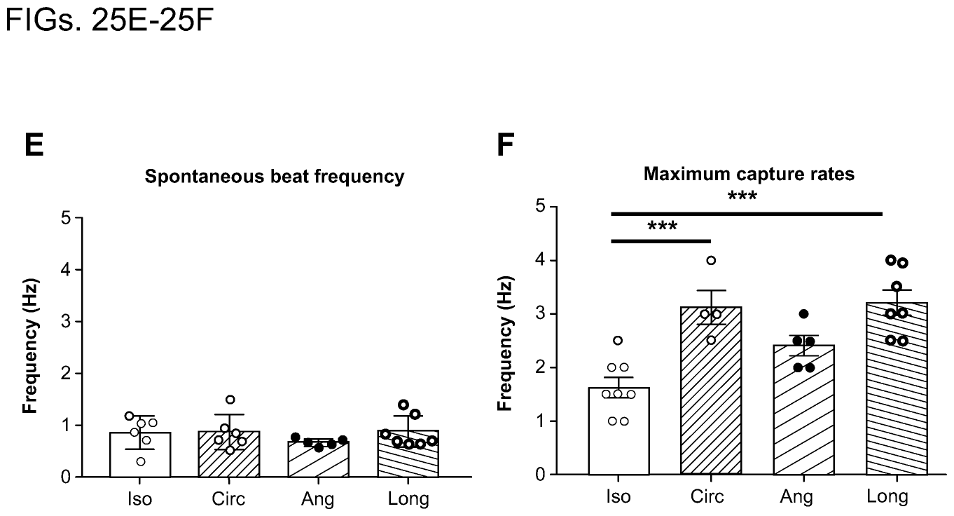

To test this hypothesis, the computational model was adapted to match the cellular remodeling observed of the circumferentially patterned tissues after 4 days in culture where the inner most layers were patterned longitudinally and the outer most layers were more isotropic. This configuration revealed that the cells interfacing with the fibrin now experienced almost three-fold less shear and in a similar pattern as the longitudinal group (FIG. 25A, blue dotted line). Similarly, the patterns of strain within the longitudinal-random configuration also adapted to mirror that of the longitudinal condition within the inner cell layers only (FIGS. 25D and 25E, blue dotted lines). There was also a sharp change in both shear stress and strain observed at the interface of the differently oriented inner and outer cells layers. However, the longitudinal shear stress at that interface is markedly less than the shear exhibited at the cell-fibrin interface of the purely circumferential condition. These results suggested that there is a threshold of perpendicular shear force that is required to promote cellular remodeling to align parallel with the direction if the opposing force.

### Example 13: Analysis of Structure-Functional Relationship via Luminal (or Internal) Pressure Production

To explore the structure-function relationships that might exist between cellular patterning and the observed remodeling effects, each tissue organization was evaluated for their ability to generate isovolumic pressures (FIG. 25A-25F). Ventricular models with biomimetic anisotropic patterning would be afforded better function than isotropic tissues. Pressure-sensing catheters were threaded into the lumens of each ventricular model after 4 days in culture and pressure readings were recorded during spontaneous or electrically paced contractions. Circumferentially and longitudinally patterned tissues performed similarly, and both generated significantly greater pressure amplitudes than isotropic tissues with random cellular organization (FIG. 25C). They were also found to have faster contraction and relaxation velocities than the isotropic and angled tissues and could be paced at higher frequencies as demonstrated by their maximum capture rates (FIG. 25D-25F). The angled tissues were found not to have significantly greater function than isotropic tissues all around, but there was an upward trend towards the functional performance of the circumferential and longitudinal groups. These findings suggest that anisotropic tissue organization would be allow for greater functional output but also demonstrated that circumferential and longitudinal tissue organizations outperformed angled ones.

### Example 14: Cell Sheet Stacking Using Cells Sheets Grown on Flexible TNFS

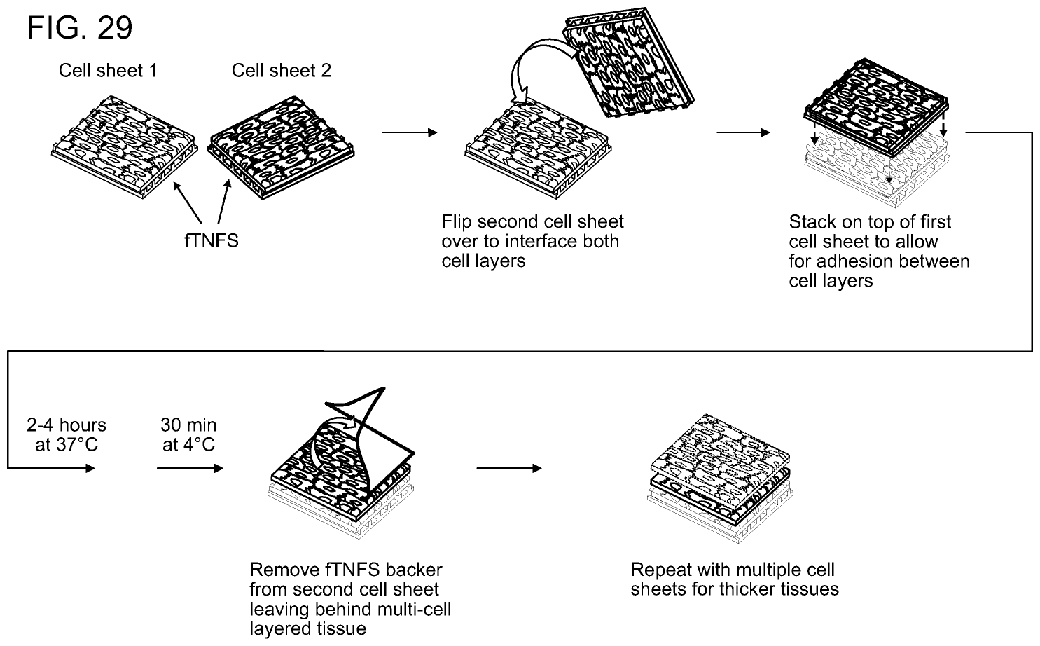

In addition to using the gel casting method described herein, another method of stacking cell sheets to form thick, multi-cell layered tissues is to invert one cell sheet and place it on top of another. This method allows the sheets to form cell-cell and cell-matrix adhesions between the two layers at physiological temperatures. Once adhesions are formed, the temperature can be lowered to promote release of the fTNFS from the top cell sheet (see FIG. 29). The fTNFS can then be peeled off, like the backing from an adhesive sticker, leaving behind a now two-layered tissue. This process could be repeated many times to create the desired thickness, e.g., thicker tissues.

### Discussion

There are few tissue engineering approaches that can recapitulate the multi-scale organization present in the myocardium, such as formation of an organized functional syncytium within a physiological tissue environment. Here, the flexible-TNFS platform was adapted to engineer human 3D cardiac ventricular models with controllable cellular architecture. The goal was to model different cellular organizations that exist within the myocardium and evaluate their structure-function relationships. Engineered ventricular models could be patterned with circumferential, angled, and longitudinal cellular organization using fTNFS and custom tissue casting molds (FIG. 22A-22J). A stark perpendicular cellular remodeling of the inner most cell layers of the circumferentially patterned tissues was observed. This remodeling effect was not exhibited by any other tissue organization (FIG. 23A-23F). Instead, the longitudinal, angled, and isotropic tissues retained their initial cellular orientation on average but with a less pronounced alignment direction. Cellular remodeling in response to mechanical stretching has been previously reported in other studies with adult rat cardiac fibroblasts¹⁷, endothelial cells¹⁸⁻²⁰, smooth muscle cells²¹, dermal fibroblasts^{22,23}; however, these studies were performed with either 2D cell monolayers or 3D laminar tissue patch settings. In the 2D settings, the cells often aligned their stress fibers perpendicularly to the principle direction of cyclic mechanical stretch as a form of “strain avoidance”¹⁸⁻²⁰. However, when cells are embedded in a 3D hydrogel, the cells align parallel to the stretch direction²⁴⁻²⁷. In the system, the cellular contraction forces provide their own mechanical stimulus and have a more complex 3D geometry than previous studies, so it is unclear which phenomenon is driving realignment. Differing patterns in shear forces or strain might be elicited by each of the initial cellular patterning schemes that motivate cellular alignment or avoidance to the direction of these forces.

The initial cellular alignment patterns were computationally modeled and tissue deformations during contraction with a custom 3D finite element model (FIG. 24A-24E). Circumferentially patterned tissues were predicted to produce large shear forces perpendicular to the cellular alignment on day 1 of culture. The observed tissue organization for the circumferential group on day 4 was modeled where the inner most layers were now longitudinally aligned and the outer layers were more isotropic (FIG. 24A-24E, dotted blue lines). The model predicted smaller shear forces and in the same direction as the longitudinally patterned tissues at the fibrin-cell interface for this remodeled configuration. These data to have two implications: (1) the initially circumferential contraction patterns produce great enough perpendicular shear forces at the fibrin-cell interface to promote cellular remodeling, and that (2) cells reorganize to avoid this perpendicular shear by aligning parallel to the direction of shear provided by upward and inward motions during contractions. With this logic, the angled and isotropic tissue organizations did not exhibit the same remodeling because strong gradients of shear stress were not present to promote their reorganization. Additionally, the longitudinally patterned tissues produced large shear forces at the cell-fibrin interface but in the same direction as the upward tissue movements during contraction, and therefore cells remained oriented longitudinally. In combination, these results are congruous with other reports of cells in 3D settings aligning with the direction of mechanical stretch²⁴⁻²⁸, but they also suggest that remodeling is simultaneously in avoidance of high shear stress.

Each model's ability to create pressure after 4 days in culture, circumferential and longitudinally patterned tissues outperformed isotropic and angled tissues and were also capable of capturing at higher pacing frequencies (FIG. 25A-25F). These results taken in consideration with the observations of longitudinal remodeling in the circumferential tissue case suggest that longitudinal tissue architecture provides functional benefit. Furthermore, longitudinal tissue organization, whether pre-patterned or spontaneously generated, was accompanied by greater pressure production, contraction and relaxation speeds, and faster pacing frequencies. This is likely due to the greater cellular alignment in the longitudinal direction overall in both the circumferential and longitudinal cases (FIGS. 24B and 24F day 4). It was interesting to observe that the angled tissues did not have significantly better function than isotropic tissues. However, the computational model suggested that they have similar patterns of shear force and strain transmurally (FIG. 25A-25F), which might explain how their functional outputs were also similar.

Overall, these results support the hypothesis that recapitulating the anisotropic organization of the myocardium within a physiological 3D tissue environment provide functional benefit compared to isotropic models. Additionally, the observed remodeling effect in response to gradients of transmural shearing forces has implications for how helical myocardial patterning might occur in cardiac morphogenesis and development. For example, the embryonic heart first begins as a circumferentially patterned tube and develops advanced helical myocardial patterning around 12-14 weeks after fertilization in humans^{29,30}. However, it is poorly understood as to how this patterning is inspired or initiated³¹. Potentially, in combination with heart tube looping, changing hemodynamic loads, and proliferative and hypertrophic growth in the embryonic heart act to set up radial or transmural patterns of stresses and strains that promote differential cellular remodeling from circumferential to helical over time. In future, the tissue engineering approach described herein could be used to study the mechanics of tissue morphogenesis and mechanotransduction in the heart and other organs.

### Materials and Methods

Fabrication of Flexible Thermoresponsive Nanofabricated Substrates (fTNFS)

To enable the production of cardiac ventricular models with anisotropic cellular organization, flexible films with nanoscale topographical cues and thermoresponsive properties were fabricated as previously described^{15,32}. Briefly, 100 μL of a UV-curable polyurethane acrylate (PUA) polymer (Norland Optical Adhesive #76) mixed with 1% w/w glycidyl methacrylate (GMA) was sandwiched and spread between a flexible polyethylene terephthalate (PET) film (5×5 cm) and a PUA master mold with nanoscale parallel ridges and grooves (800×800×600 nm, w×h×d). The PUA-GMA polymer mixture was flash cured under high-intensity UV light (365 nm) and the flexible film now with nanoscale features was removed from the master mold and placed under low-intensity UV bulbs overnight for final curing. The nanopatterned flexible films were then washed with an amine-terminated poly(N-isopropylacrylamide) (pNIPAM) solution (13 μM in H₂O, Mₙ=2500 Sigma-Aldrich) for 24 hours on a tabletop rocker (55 rpm, room temperature) to provide thermoresponsive surface functionalization. The flexible thermoresponsive nanofabricated substrates (fTNFS) were then rinsed in deionized water (DI-H₂O) to remove excess pNIPAM).

Flexible TNFS were cut into fan shapes (radius=12 mm, θ=135°, area=1.17 cm²) using a die cutter to produce substrates with nanogrooves aligned at either a 0°, 45°, or 90° angle relative to the x-axis (FIG. 22B). Cut fTNFS were affixed to custom fan-shaped polydimethylsiloxane wells (PDMS, Sylgard 181) using a UV curable adhesive (Norland Optical Adhesive #83H). Wells were made only 10% larger in dimension than the fTNFS to control the cell seeding area and minimize superfluous cell waste. Culture wells were rinsed with DI-H₂O before UV sterilization (294 nm) for 4+ hours in a biosafety cabinet. Sterilized culture wells were treated with fetal bovine serum (FBS, Sigma) overnight at 37° C. before cell seeding to promote cellular attachment.

### Pluripotent Stem-Cell Culture and Differentiation

A human urine-derived induced pluripotent stem cell line (hiPSC UC 3-4, wild-type, male) was used for differentiation of cardiomyocytes (CMs) and endocardial-like endothelial cells (ECs)³³. Production of CMs and ECs was performed using well established monolayer-based directed differentiation protocols^{34,35}. Briefly, hiPSC colonies were expanded to 80% confluency on Matrigel-coated plates (1:60, Corning), dissociated, and re-plated at either a high (270 k/cm²) or low (100 k/cm²) density for directed differentiation of CMs or ECs, respectively. High-density monolayers were cultured for 48 hours in mTeSR medium (STEMCELL Technologies) before induction (day 0) of mesoderm specification with 10 μm CHIR-99021 (Fischer Technologies) in Roswell Park Memorial Institute 1640 (RPMI) medium with B27 supplement without insulin (Gibco). To further specify cardiomyocyte lineage, high-density monolayers were exposed to the Wnt-inhibitor IWP4 (Stemgent) on day 3 in RPMI+B27 without insulin and cultured with RPMI+B27 with insulin from day 7 onwards. To purify differentiation populations for CMs, cardiac differentiated cultures replated and exposed to a glucose-poor and lactose-rich medium (RPMI 1640 without glucose or L-glutamine supplemented with 4 mM lactate) on day 14 for two days or until only beating cells remained. Cells were harvested on day 17 or later and stained for cardiac-specific markers using a fluorescently conjugated antibody (anti cardiac troponin T (cTnT)—Alexa fluor 488, 1:100, Thermo-Scientific) for flow cytometry. Only populations of ≥95% cTnT− positivity were used for this study.

Endothelial cells were similarly differentiated from low-density (100 k/cm²) hiPSC monolayers plated in mTeSR medium with 1 μm CHIR-99021. After 24 hours, cells were induced with activin-A (R&D Systems) and Matrigel (1:60) in RPMI+B27 for 18 hours. The cells were then cultured with bone morphogenic protein-4 (BMP-4; R&D Systems) and CHIR-99021 in RPMI-B27 medium to specify for cardiac mesoderm lineages. To further select for cardiac endothelial populations, cells were incubated with StemPro-34 medium from days 2 to 5 with a cocktail of growth factors: vascular endothelial growth factor (VEGF; PeproTech), BMP-4, basic fibroblast growth factor (bFGF; R&D Systems), ascorbic acid, and monothioglycerol. On day 5, monolayers were dissociated and re-plated at a lower density (13 k/cm2) on 0.1% gelatin-coated plates in Endothelial Growth Medium-2 (EGM-2, Lonza) supplemented with VEGF, bFGF, and CHIR-99021 until day 12. EC population purity was evaluated on day 12 via live-cell flow cytometry using an anti-CD31—Alex 488 conjugated antibody (1:100, R&D Systems). Only EC populations with ≥90% CD31− positivity were used for this study.

Serial-Seeding of fTNFS with hiPSC-Derived CMs and ECs

To eliminate confounding factors of cell age and maturation, CMs and ECs used between days 17-25 and 12-14, respectively. CMs and ECs were dissociated separately and mixed together such that final population was 89% CMs and 11% ECs CMs:ECs), as previously described¹⁵. The cell mixture was seeded onto FBS-treated fTNFS between 175 and 185 k/cm2 in 120 μL of cardiac growth medium (75% RPMI-B27+insulin, 25% EGM-2, 10% FBS, 1% penicillin/streptomycin) to form a highly confluent monolayer (day 0). The cell mixture was cultured overnight at 37° C., 5% CO₂ to allow for maximum cell adhesion to the fTNFS, mechanosensation of the nanotopography, and cellular elongation along the nanogrooves and ridges (FIG. 22B). 18-24 hours after the first seeding event (day 1), additional CMs and ECs were dissociated and mixed again at a 7:1 ratio as described above. The excess medium and non-adherent cells were aspirated from the fTNFS surface and replaced with 120 μL of a second CM-EC cell suspension to provide another layer of cells between 175 and 185 k/cm². The twice-seeded or serial-seeded fTNFS was cultured overnight at 37° C., 5% CO₂ to allow for cell-cell adhesion to occur between the first and second seeded layers before addition of 2 mL of warmed (37° C.) cardiac growth medium (day 2). Serial-seeded cell layers were cultured for an additional 4-5 days to allow for formation of aligned cardiac sheets with coordinated beating patterns before use in fabrication of 3D ventricular models.

### Custom Molds for Fabrication of 3D Ventricle Models

Modular 3D-printed molds were designed in a computer aided design software (Solidworks, Autodesk) and fabricated using a 3D-printer (CUBICON Style) and acrylonitrile butadiene styrene filament (Makerbot). The mold pieces were printed with a 0.1 mm line thickness and brushed with acetone before use to minimize the ridges formed by the layer-by-layer printing process. A modular design was incorporated to aid in mold disassembly and tissue extraction after fabrication (FIG. 26). The assembled molds contained a hollow conical lumen (Diameter=6 mm, H=7 mm) which was used to cast a tissue with a conical geometry that is similar to the left-ventricle of the heart. The final tissue product was on scale to a whole mouse heart¹⁶.

Fabrication of 3D Cardiac Ventricular Models from Organized Cardiac Sheets

Organized cardiac sheets with spontaneous and synchronous beating patterns were formed after five days in culture on the fTNFS. Before tissue casting, 3D-printed mold pieces were pre-sterilized with 70% ethanol and submerged in hydrophobic Pluronic F-127 (5% in DI-water, Sigma) for at least 20 minutes to prevent the tissues from attaching to the molds. The submerged pieces were removed and allowed to dry in a sterile biosafety cabinet (BSC) for at least 5 minutes before assembly and tissue casting. Meanwhile, cardiac sheets were incubated with room-temperature phosphate buffer saline (PBS, Sigma) for 10 minutes to initiate partial cell sheet detachment from the fTNFS. The fTNFS was removed from the culture dishes using forceps to grasp both corners of the flexible pattern. Carefully, the two opposing corners were brought together and overlapped to create cone shape with the cell layers facing inwards (FIG. 22C) and inserted into the complementary conical mold. One pair of forceps was used to hold the fTNFS in the mold while another was used to assemble the remaining pieces and fasten the fTNFS in place. To create a fibrin hydrogel scaffold, 18 μL of thrombin (50 units/mL, Sigma) was mix with 300 μL of fibrinogen (20 mg/mL, Sigma) and 200 μL of the thrombin/fibrinogen mixture was quickly pipetted into the open mold as to fill the entire well (FIG. 26, .iv). Finally, the top mold piece was inserted through the mold's opening and into the conical well, pushing excess fibrin out and causing it to flow into the remaining negative space of the mold. This overflow was essential for attaching the final casted tissue onto the tissue mount for future culture purposes.

The fully assembled mold containing the fTNFS and cell sheets was placed into a humidified 37° C. incubator for 1 hour to allow for the thrombin/fibrinogen mixture to fully polymerize into a fibrin hydrogel scaffold within the mold. The top and bottom portions of the mold were then removed and the remaining mold-fTNFS-cell sheet assembly was then submerged in cardiac growth medium and cultured overnight at 37° C., 5% CO₂ to allow for the cell sheets to adhere to the newly polymerized fibrin hydrogel scaffold. After incubation, the mold was submerged in cold (4° C.) medium and incubated at 4° C. for 20 minutes to promote complete cell sheet detachment from the fTNFS. The mold was then fully disassembled and the fTNFS were removed leaving behind a hollow, ventricle-shaped tissue and organized cell sheets wrapped around the outside walls of the fibrin hydrogel scaffold (FIGS. 22C and 22D). The tissues were placed into 6 well plates with 9 mL of fresh medium for further culture.

### Tissue Culture and Electrical Field Stimulation

After tissue casting (day 0) and extraction from the molds (day 1), ventricular models were cultured for an additional 24 hours before proving electrical field stimulation on days 2-4. On day 2, tissues were exposed to a 1 Hz pacing frequency (10 millisecond pulses, 3 V) for 24 hours and then increased to 1.5 Hz for an additional 1-2 days before functional measurements were taken on days 3-5 of culture.

### Functional Assessment of Ventricular Models

After 4 days in culture, ventricular models were functionally evaluated using a pressure-sensing catheter. First, the ventricular models were transferred onto a custom 3D-printed stand within a 6 well-plate such that the tissues were positioned vertically with the base and the opening of the tissue lumen were at the highest point and the apex hung below. The wells were filled with warmed Tyrode's solution (140 mM NaCl, 5 mM KCl, 5 mM HEPES, 1 mM NaH₂PO₄, pH 7.4) and a thin PDMS cover was placed over the opening of the tissue's lumen at the base to create a closed-volume system. A small x-shaped slit was previously cut into the PDMS gasket to allow for the tip of a Millar pressure sensing catheter (model SPR-671) to be threaded into the lumen of the tissue (FIG. 25A). Spontaneous pressure recordings were taken of each tissue to evaluate a baseline beat frequency using the Lab Chart Pro software (ADI Instruments). Platinum stimulating electrodes (ø=0.5 mm) were then placed on either side of the tissue stand (15 mm apart) and the tissues were paced at 1 Hz with 10 ms pulses at 10 V. Pressure recordings were taken at 1 Hz for one minute before the pacing frequency was increased by 0.5 Hz and pressure production was recorded for another minute. This incremental pacing scheme was continued until the tissue could no longer capture at the challenging pacing frequency.

### Analysis of Pressure Production Data

Spontaneous and electrically paced pressure recording events were parsed and exported from the LabChart Pro software as .csv files and imported into MATLAB (MathWorks) for analysis. A custom MATLAB script was used to find maximum peaks within each dataset and locate the preceding troughs to find the minimum peaks. The amplitudes of the minimums were subtracted from the maximum pressure peaks to calculate a pressure amplitude. The average pressure values at a 1 Hz pacing frequency for each tissue group (isotropic, patterned 0°, 45°, 90°) were averaged and compared using a one-way ANOVA and a Tukey's multiple comparisons test (alpha=0.05) (FIG. 25C). Similarly, spontaneous and maximum capture rates for each group were averaged and compared using a one-way ANOVA and a Tukey's multiple comparisons test (alpha=0.05) (FIGS. 25E and 25F).

Tissues were also evaluated for their contractility through their ability to generate pressure over time during systole and diastole, or dP/dt. In the LabChart software, the first derivative of the raw pressure signal (dP/dt) was calculated for each tissue under each pacing frequency and averaged over the one-minute trace. The data were exported as a .csv file and imported into Prism where the average contraction and relaxation values for each group (isotropic, patterned 0°, 45°, 90°) were compared using a two-way ANOVA and Tukey's multiple comparisons test (alpha=0.05).

### Instron Compression Testing of Fibrin

Compressive moduli of hydrated and crosslinked fibrin hydrogels (20 mg/mL) were measured using an Instron 5900 Series Universal Testing System equipped with a 10 N static load cell. Samples 5 mm in height and 6.8 mm in diameter were compressed at a rate of 10 mm/min until failure.

### Finite Element Analysis

An axisymmetric finite element model of the conical tissue was built in ANSYS to understand the realignment of tissues observed experimentally. The geometry shown in FIG. 4 consisted of an inner fibrin layer 250 microns thick and an outer cardiomyocyte cell layer 70 microns thick. The inner diameter at the base was 5 mm and the length of the tissue from base to apex was 7 mm. A 2-degree section of the model was meshed with 32,026 nodes and 4,390 quadratic 3D solid elements (SOLID186 in ANSYS), and cyclic periodicity was applied with 180 repeats to model the full conical shape. Considering the small deformations of the conical tissues observed experimentally, an isotropic elasticity was applied to both the fibrin and cell layers with a Young's modulus of 6.6 kPa and 20 kPa, respectively, and a nearly incompressible Poisson's ratio of 0.49. Fibrin stiffness was determined experimentally through compression testing, as described above. To simulate contraction of the cardiomyocyte cell layer, a negative strain was applied to the cell layer causing either unidirectional or isotropic contraction. The direction of the contractile strain was rotated to replicate all experimental orientations, and the amount of contractile strain was calibrated by modeling the longitudinal orientation tissues and matching the apex displacement of the model to the one-day old experimental data.

### Immunofluorescent Staining and Confocal Imaging

Tissues were fixed in 4% paraformaldehyde for 24 hours at 4° C. before immunocytochemistry was performed. Tissues were permeabilized in a phosphate buffered saline (PBS) solution with 0.2% Triton-X 100 (Sigma-Aldrich, 9002-93-1), 5% goat serum, and 0.5% bovine serum albumen (BSA, Sigma-Aldrich A7906) for one hour at room temperature. After three, five-minute PBS washes, the tissues were incubated with an antigen blocking buffer (1.5% goat serum, 0.2% Triton-X 100) for two hours at room temperature to minimize non-specific antibody binding. Primary antibodies specific to sarcomeric titin (Myomedix, 1:300) were applied in a staining solution (0.2% Triton-X, 1.5% goat serum) and incubated overnight at 4° C. Excess primary antibodies then were removed through three serial PBS washes before the secondary antibody (Alexa Fluor 647 donkey anti rabbit, 1:200) and fluorescently conjugated phalloidin (Invitrogen A12379, 1:200) were added. Tissues were incubated with secondary antibodies for two hours at room temperature in the dark before staining for nuclei DAPI (Invitrogen, D1306).

To aid in visualization of the large tissue surface area, the stained ventricular models were gently flattened and sandwiched between two glass coverslips using a think (˜3 mm) PDMS gasket with Vectashield anti-fade mounting medium (Vectro Laboratories, H-1000-10). The tissues were then imaged using low- and high-powered objectives (20× air, 40× water immersion lens) and a SP8 Leica confocal microscope. Large-area stitched z-stacks were taken of the entire visible tissue area using the 20×-magnification objective. For more detailed analysis of the cellular and cytoskeletal structure through the tissue walls, 40×-magnification z-stacks were taken at several locations across the tissue.

### Analysis of Cellular Organization

Several tissues within each group were fixed and stained at day 0 and day 4 of culture as described above. Several confocal z-stacks were taken at 40× magnification across the tissue surface to survey transmural cellular alignment in the top (base), middle, and bottom (apex) sections. The z-stacks were parsed into halves (˜35 μm thick) to separate out the outer and inner cell layers as defined by the nuclear counter-stain (FIG. 22F). These sections were used to create maximum intensity projections (MIPs) representing the outer cell layers closest to the surface and inner cell layers closest to the fibrin wall of the tissue (FIG. 23A-23F). Cellular alignment was determined for each of these MIPs based on alignment of the filamentous actin (F-actin) cytoskeleton using a modified MATLAB script as previously described^{15,32,36}. Briefly, a low-pass Gaussian filter and edge detection to create a 2D convolution from which vertical and horizontal edges are detected using a Sobel filter³⁷. These vectors are then used to calculate intensity gradient magnitudes across each pixel within an image. The images are processed by thresholding to define the edges of single cells or groups of cells and calculate their orientation angle between −90° and +90° relative to the x-axis at 0°. The total orientation angles detected within the image are binned and normalized using the probability density function in MATLAB (normpdf).

To quantify the principle alignment direction for the inner and outer cell layers of each tissue, the circular means for multiple images from each cell layer were calculated using the Circular Statistics MATLAB toolbox. The mean orientations for the inner and outer layers of each tissue were then grouped and averaged (FIG. 22E). The principle angles of alignment for each layer and tissue group were compared on days 0 and 4 using a parametric Watson-Williams multi-sample test for circular data (circ_wwtest function) and Tukey-Kramer multiple comparisons (alpha=0.05).

To determine the strength of cellular alignment around the principle orientation, w the resultant vector length (RVL) values for each image's distribution was calculated using the Circular Statistics Toolbox (MATLAB, circ_r function). For example, if the distribution of angular data around the mean is very concentrated a RVL closer to one will result, whereas if the distribution is wide a RVL closer to zero will result. RVLs were interpreted closer to one to mean that the cells were highly aligned in the principle direction whereas a RVL closer to zero would indicated the cells were less aligned in in the principle direction. Average RVL values of the inner and outer layers were compared within each group on days 0 and 4 using a one-way ANOVA with Tukey's multiple comparisons (alpha=0.05).

### REFERENCES

[1] A. Patel, B. Fine, M. Sandig, K. Mequanint, Elastin biosynthesis: the missing link in tissue-engineered blood vessels, Cardiovasc. Res. 71 (2006) 40-49.

[2] U. Roostalu, J. K. Wong, Arterial smooth muscle dynamics in development and repair, Dev. Biol. 435 (2018) 109-121.

[3] V. Domenga, P. Fardoux, P. Lacombe, M. Monet, J. Maciazek, L. T. Krebs, B. Klonjkowski, E. Berrou, M. Mericskay, Z. Li, E. Tournier-Lasserve, T. Gridley, A. Joutel, Notch 3 is required for arterial identity and maturation of vascular smooth muscle cells, Genes Dev. 18 (2004) 2730-2735.

[4] H. M. Phillips, H. J. Rhee, J. N. Murdoch, V. Hildreth, J. D. Peat, R. H. Anderson, A. J. Copp, B. Chaudhry, D. J. Henderson, Disruption of planar cell polarity signaling results in congenital heart defects and cardiomyopathy attributable to early cardiomyocyte disorganization, Circ. Res. 101 (2007) 137-145.

[5] B. M. Weinstein, D. L. Stemple, W. Driever, M. C. Fishman, gridlock, a localized heritable vascular patterning defect in the zebrafish, Nat. Med. 1 (1995) 1143-1147.

[6] K. Ronaldson-Bouchard, S. P. Ma, K. Yeager, T. Chen, L. Song, D. Sirabella, K. Morikawa, D. Teles, M. Yazawa, G. Vunjak-Novakovic, Advanced maturation of human cardiac tissue grown from pluripotent stem cells, Nature 556 (2018) 239-243.

[7] B. Liau, N. Christoforou, K. W. Leong, N. Bursac, Pluripotent stem cell-derived cardiac tissue patch with advanced structure and function, Biomaterials 32 (2011) 9180-9187.

[8] S. S. Nunes, J. W. Miklas, J. Liu, R. Aschar-Sobbi, Y. Xiao, B. Zhang, J. Jiang, S. Masse, M. Gagliardi, A. Hsieh, N. Thavandiran, M. A. Laflamme, K. Nanthakumar, G. J. Gross, P. H. Backx, G. Keller, M. Radisic, Biowire: a platform for maturation of human pluripotent stem cell-derived cardiomyocytes, Nat. Methods 10 (2013) 781-787.

[9] Z. H. Syedain, L. A. Meier, M. T. Lahti, S. L. Johnson, R. T. Tranquillo, Implantation of completely biological engineered grafts following decellularization into the sheep femoral artery, Tissue Eng. 20 (2014) 1726-1734.

[10] C. Quint, M. Arief, A. Muto, A. Dardik, L. E. Niklason, Allogeneic human tissueengineered blood vessel, J. Vasc. Surg. 55 (2012) 790-798.

[11] S. L. M. Dahl, A. P. Kypson, J. H. Lawson, J. L. Blum, J. T. Strader, Y. Li, R. J. Manson, W. E. Tente, L. DiBernardo, M. T. Hensley, R. Carter, T. P. Williams, H. L. Prichard, M. S. Dey, K. G. Begelman, L. E. Niklason, Readily available tissue-engineered vascular grafts, Sci. Transl. Med. 3 (2011) 68ra9.

[12] A. Skardal, J. Zhang, G. D. Prestwich, Bioprinting vessel-like constructs using hyaluronan hydrogels crosslinked with tetrahedral polyethylene glycol tetracrylates, Biomaterials 31 (2010) 6173-6181.

[13] T. J. Hinton, Q. Jallerat, R. N. Palchesko, J. H. Park, M. S. Grodzicki, H.-J. Shue, M. H. Ramadan, A. R. Hudson, A. W. Feinberg, Three-dimensional printing of complex biological structures by freeform reversible embedding of suspended hydrogels, Sci. Adv. 1 (2015) e1500758.

[14] A. Lee, A. R. Hudson, D. J. Shiwarski, J. W. Tashman, T. J. Hinton, S. Yemeni, J. M. Bliley, P. G. Campbell, A. W. Feinberg, 3D bioprinting of collagen to rebuild components of the human heart, Science 80 (365) (2019) 482-487.

[15] A. K. Miri, A. Khalilpour, B. Cecen, S. Maharjan, S. R. Shin, A. Khademhosseini, Multiscale bioprinting of vascularized models, Biomaterials 198 (2019) 204-216.

[16] A. Jiao, N. E. Trosper, H. S. Yang, J. Kim, J. H. Tsui, S. D. Frankel, C. E. Murry, D.-H. Kim, Thermoresponsive nanofabricated substratum for the engineering of threedimensional tissues with layer-by-layer architectural control, ACS Nano 8 (2014) 4430-4439.

[17] A. Jiao, C. T. Moerk, N. Penland, M. Perla, J. Kim, A. S. T. Smith, C. Murry, D.-H. Kim, Regulation of skeletal myotube formation and alignment by nanotopographically controlled cell-secreted extracellular matrix, J. Biomed. Mater. Res. (2018).

[18] N. Penland, E. Choi, M. Perla, J. Park, D.-H. Kim, Facile fabrication of tissue-engineered constructs using nanopatterned cell sheets and magnetic levitation, Nanotechnology 28 (2017) 075103.

[19] A. Callegari, M. L. Coons, J. L. Ricks, M. E. Rosenfeld, M. Scatena, Increased calcification in osteoprotegerin-deficient smooth muscle cells: dependence on receptor activator of NF-κB ligand and interleukin 6, J. Vasc. Res. 51 (2014) 118-131.

[20] X. Guan, D. L. Mack, C. M. Moreno, J. L. Strande, J. Mathieu, Y. Shi, C. D. Markert, Z. Wang, G. Liu, M. W. Lawlor, E. C. Moorefield, T. N. Jones, J. A. Fugate, M. E. Furth, C. E. Murry, H. Ruohola-Baker, Y. Zhang, L. F. Santana, M. K. Childers, Dystrophindeficient cardiomyocytes derived from human urine: new biologic reagents for drug discovery, Stem Cell Res. 12 (2014) 467-480.

[21] X. Lian, J. Zhang, S. M. Azarin, K. Zhu, L. B. Hazeltine, X. Bao, C. Hsiao, T. J. Kamp, S. P. Palecek, Directed cardiomyocyte differentiation from human pluripotent stem cells by modulating Wnt/β-catenin signaling under fully defined conditions, Nat. Protoc. 8 (2012) 162-175.

[22] N. J. Palpant, L. Pabon, M. Roberts, B. Hadland, D. Jones, C. Jones, R. T. Moon, W. L. Ruzzo, I. Bernstein, Y. Zheng, C. E. Murry, Inhibition of -catenin signaling respecifies anterior-like endothelium into beating human cardiomyocytes, Development 142 (2015) 3198-3209.

[23] N. J. Palpant, L. Pabon, C. E. Friedman, M. Roberts, B. Hadland, R. J. Zaunbrecher, I. Bernstein, Y. Zheng, C. E. Murry, Generating high-purity cardiac and endothelial derivatives from patterned mesoderm using human pluripotent stem cells, Nat. Protoc. 12 (2017) 15-31.

[24] N. Hemmi, S. Tohyama, K. Nakajima, H. Kanazawa, T. Suzuki, F. Hattori, T. Seki, Y. Kishino, A. Hirano, M. Okada, R. Tabei, R. Ohno, C. Fujita, T. Haruna, S. Yuasa, M. Sano, J. Fujita, K. Fukuda, A massive suspension culture system with metabolic purification for human pluripotent stem cell-derived cardiomyocytes, Stem Cells Transl. Med. 3 (2014) 1473-1483.

[25] K. Nagase, M. Yamato, H. Kanazawa, T. Okano, Poly(N -isopropylacrylamide)-based thermoresponsive surfaces provide new types of biomedical applications, Biomaterials 153 (2018) 27-48.

[26] H. Takahashi, T. Okano, Thermally-triggered fabrication of cell sheets for tissue engineering and regenerative medicine, Adv. Drug Deliv. Rev. (2019).

[27] H. Cho, H. Jonsson, K. Campbell, P. Melke, J. W. Williams, B. Jedynak, A. M. Stevens, A. Groisman, A. Levchenko, Self-organization in high-density bacterial colonies: efficient crowd control, PLoS Biol. 5 (2007) e302.

[28] D.-H. Kim, C.-H. Seo, K. Han, K. W. Kwon, A. Levchenko, K.-Y. Suh, Guided cell migration on microtextured substrates with variable local density and anisotropy, Adv. Funct. Mater. 19 (2009) 1579-1586.

[29] G. K. Owens, M. S. Kumar, B. R. Wamhoff, Molecular regulation of vascular smooth muscle cell differentiation in development and disease, Physiol. Rev. 84 (2004) 767-801.

[30] Y. Chen, Y. Lin, K. M. Davis, Q. Wang, J. Rnjak-Kovacina, C. Li, R. R. Isberg, C. A. Kumamoto, J. Mecsas, D. L. Kaplan, Robust bioengineered 3D functional human intestinal epithelium, Sci. Rep. 5 (2015) 13708.

[31] L. A. MacQueen, S. P. Sheehy, C. O. Chantre, J. F. Zimmerman, F. S. Pasqualini, X. Liu, J. A. Goss, P. H. Campbell, G. M. Gonzalez, S.-J. Park, A. K. Capulli, J. P. Ferrier, T. F. Kosar, L. Mahadevan, W. T. Pu, K. K. Parker, A tissue-engineered scale model of the heart ventricle, Nat. Biomed. Eng. 1 (2018).

[32] S. R. Finkbeiner, J. J. Freeman, M. M. Wieck, W. El-Nachef, C. H. Altheim, Y. H. Tsai, S. Huang, R. Dyal, E. S. White, T. C. Grikscheit, D. H. Teitelbaum, J. R. Spence, Generation of tissue-engineered small intestine using embryonic stem cell-derived human intestinal organoids, Biol. Open. 4 (2015) 1462-1472.

[33] T. K. Merceron, M. Burt, Y.-J. Seol, H.-W. Kang, S. J. Lee, J. J. Yoo, A. Atala, A 3D bioprinted complex structure for engineering the muscle-tendon unit, Biofabrication 7 (2015) 035003.

[34] D. Sasaki, K. Matsuura, H. Seta, Y. Haraguchi, T. Okano, T. Shimizu, Contractile force measurement of human induced pluripotent stem cell-derived cardiac cell sheet-tissue, PloS One 13 (2018) e0198026.

[35] K. Sakaguchi, T. Shimizu, T. Okano, Construction of three-dimensional vascularized cardiac tissue with cell sheet engineering, J. Contr. Release 205 (2015) 83-88.

[36] N. Juthani, C. Howell, H. Ledoux, I. Sotiri, S. Kelso, Y. Kovalenko, A. Tajik, T. L. Vu, J. J. Lin, A. Sutton, J. Aizenberg, Infused polymers for cell sheet release, Sci. Rep.

[37] Y. Haraguchi, T. Shimizu, M. Yamato, T. Okano, Scaffold-free tissue engineering using cell sheet technology, RSC Adv. (2012).

[38] M. Tiburcy, J. E. Hudson, P. Balfanz, S. Schlick, T. Meyer, M.-L. Chang Liao, E. Levent, F. Raad, S. Zeidler, E. Wingender, J. Riegler, M. Wang, J. D. Gold, I. Kehat, E. Wettwer, U. Ravens, P. Dierickx, L. W. van Laake, M. J. Goumans, S. Khadjeh, K. Toischer, G. Hasenfuss, L. A. Couture, A. Unger, W. A. Linke, T. Araki, B. Neel, G. Keller, L. Gepstein, J. C. Wu, W.-H. Zimmermann, Defined engineered human myocardium with advanced maturation for applicatons in heart failure modeling and repair, Circulation 135 (2017) 1832-1847.

[39] R. A. Li, W. Keung, T. J. Cashman, P. C. Backeris, B. V. Johnson, E. S. Bardot, A. O. T. Wong, P. K. W. Chan, C. W. Y. Chan, K. D. Costa, Bioengineering an Electro-Mechanically Functional Miniature Ventricular Heart Chamber from Human Pluripotent Stem Cells, Biomaterials, 2018.

[40] S. M. Maffioletti, S. Sarcar, A. B. H. Henderson, I. Mannhardt, L. Pinton, L. A. Moyle, H. Steele-Stallard, O. Cappellari, K. E. Wells, G. Ferrari, J. S. Mitchell, G. E. Tyzack, V. N. Kotiadis, M. Khedr, M. Ragazzi, W. Wang, M. R. Duchen, R. Patani, P. S. Zammit, D. J. Wells, T. Eschenhagen, F. S. Tedesco, Three-dimensional human iPSC-derived artificial skeletal muscles model muscular dystrophies and enable multilineage tissue engineering, Cell Rep. 23 (2018).

[41] W. Zhou, Y. Chen, T. Roh, Y. Lin, S. Ling, S. Zhao, J. D. Lin, N. Khalil, D. M. Cairns, E. Manousiouthakis, M. Tse, D. L. Kaplan, Multifunctional bioreactor system for human intestine tissues, ACS Biomater. Sci. Eng. 4 (2018) 231-239.

[42] T. Matsumoto, J.-I. Sasaki, E. Alsberg, H. Egusa, H. Yatani, T. Sohmura, Threedimensional cell and tissue patterning in a strained fibrin gel system, PloS One 2 (2007) e1211.

[43] B. Xu, A. Magli, Y. Anugrah, S. J. Koester, R. C. R. Perlingeiro, W. Shen, Nanotopography-responsive myotube alignment and orientation as a sensitive phenotypic biomarker for duchenne muscular dystrophy, Biomaterials 183 (2018) 54-66.

[44] H. Fujita, T. Nedachi, M. Kanzaki, Accelerated de novo sarcomere assembly by electric pulse stimulation in C2C12 myotubes, Exp. Cell Res. 313 (2007) 1853-1865.

[45] P. G. De Deyne, Formation of sarcomeres in developing myotubes: role of mechanical stretch and contractile activation, Am. J. Physiol. Physiol. 279 (2000) C1801—C1811.

[46] M. Hara, K. Tabata, T. Suzuki, M.-K. Q. Do, W. Mizunoya, M. Nakamura, S. Nishimura, S. Tabata, Y. Ikeuchi, K. Sunagawa, J. E. Anderson, R. E. Allen, R. Tatsumi, Calcium influx through a possible coupling of cation channels impacts skeletal muscle satellite cell activation in response to mechanical stretch, Am. J. Physiol. Physiol. 302 (2012) C1741—C1750.

[47] S. T. Cooper, A. L. Maxwell, E. Kizana, M. Ghoddusi, E. C. Hardeman, I. E. Alexander, D. G. Allen, K. N. North, C2C12 Co-culture on a fibroblast substratum enables sustained survival of contractile, highly differentiated myotubes with peripheral nuclei and adult fast myosin expression, Cell Motil Cytoskeleton 58 (2004) 200-211.

[48] Physiologic force-frequency response in engineered heart muscle by electromechanical stimulation, Biomaterials 60 (2015) 82-91 BIOMATERIALS.2015.03.055.

[49] M. Radisic, H. Park, H. Shing, T. Consi, F. J. Schoen, R. Langer, L. E. Freed, G. Vunjak-Novakovic, Functional assembly of engineered myocardium by electrical stimulation of cardiac myocytes cultured on scaffolds, Proc. Natl. Acad. Sci. U.S.A. 101 (2004) 18129-18134.

[50] D. D. Streeter, D. L. Bassett, An engineering analysis of myocardial fiber orientation in pig's left ventricle in systole, Anat. Rec. 155 (1966) 503-511.

[51] A. J. Pope, G. B. Sands, B. H. Smaill, I. J. LeGrice, Three-dimensional transmural organization of perimysial collagen in the heart, Am. J. Physiol. Cell Physiol. 295 (2008) H1243—H1252.

[52] D. J. H. Ae, R. H. Anderson, The Development and Structure of the Ventricles in the Human Heart, (n.d.).

[53] H. Sekine, T. Shimizu, K. Sakaguchi, I. Dobashi, M. Wada, M. Yamato, E. Kobayashi, M. Umezu, T. Okano, In vitro fabrication of functional three-dimensional tissues with perfusable blood vessels, Nat. Commun. 4 (2013).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

1. A method for making a tissue engineering scaffold, the method comprising:
layering at least one sheet or layer of cells onto a flexible scaffold, casting at least one sheet or layer of cells into a geometry, and thereby creating the tissue engineering scaffold.

2. The method of claim 1 wherein the at least one sheet of cells comprise at least an area of patterned or structurally organized cells.

3. The method of claim 1, wherein the flexible scaffold comprises a thermoresponsive material.

4. The method of claim 3 wherein the flexible scaffold detaches at about 32° C., and at greater than about 32° C. the flexible scaffold attaches.

5. (canceled)

6. The method of claim 1, wherein the sheet of cells comprises a monolayer of the cells.

7. The method of claim 1, wherein cells are aligned in a uniform direction in at least a portion of at least one sheet of cells.

8. (canceled)

9. The method of claim 1, wherein the cells comprise a muscle cell.

10. The method of claim 1, wherein the cells comprise smooth muscle cells, cardiac cells, skeletal cells, neuronal cells, cancer cells, endothelial cells, epithelial cells, fibroblasts, chondrocytes, and combinations thereof.

11. The method of claim 1, wherein the flexible scaffold is capable of being twisted, folded, stacked, rolled, or wrapped.

12. (canceled)

13. A tissue engineering scaffold capable of inducing a cell attached to the tissue engineering scaffold, wherein the tissue engineering scaffold comprises:
a flexible scaffold,
a functional layer, where the functional layer comprises poly (N-isopropylacrylamide) (pNIPAM), or a derivative thereof,
and a polymer

14. (canceled)

15. The tissue engineering scaffold of claim 13, wherein the polymer comprises an ultraviolet-curable polymer.

16. The tissue engineering scaffold of claim 13, wherein the scaffold comprises a hydrogel.

17. The tissue engineering scaffold of claim 13, wherein the cell comprises smooth muscle cells, cardiac cells, skeletal cells, neuronal cells, cancer cells, endothelial cells, epithelial cells, fibroblasts, chondrocytes, and combinations thereof.

18. The tissue engineering scaffold of claim 13, further comprising a drug molecule, an adhesion molecule, a signaling molecule, an imaging agent

19. The tissue engineering scaffold of claim 13, wherein the functional layer comprises poly (N-isopropylacrylamide) (pNIPAM).

20. A method for repair or replacement of tissue comprising:
providing tissue that has been obtained or is obtainable from a scaffold of claim 1;
administering the tissue to a patient in need thereof.

21. The method of claim 20 wherein the tissue is administered to a targeted site of the patient.

22. The method of claim 20 wherein the tissue has been detached from a flexible substrate before administering the tissue to the patient.

23. (canceled)

24. The method of claim 21 wherein the one or more additional agents comprise one or more of growth factors, small molecule therapeutic and/or lipids.

25. A method for repair or replacement of a tissue comprising applying a tissue engineering scaffold, wherein the tissue engineering scaffold comprises:
a flexible scaffold,
and a polymer, or
A method for in vitro disease modeling, comprising making a tissue engineering scaffold by layering at least one sheet of aligned cells onto a flexible scaffold, casting the sheets into geometries, and thereby creating the tissue engineering scaffold, and thereby modeling the disease of interest.

26-27. (canceled)
